SEQUENCE LISTING (5 sequences)

SEQ ID NO: 1 - protein, 157 aa, synthetic construct
DLEKIVKKIMHRAQTLLKCERCSVLLLEDIESPVVKFTKSFELMSPKCSADAENSFKESVEKSSYSDWLINNSIAELVASTGLPVNVSDAYQDPRFDAEADQISGFHIRSVLCVPIWNSNHQIIGVAQVLNRLDGKPFDDADQRLFEAFVIFCGLGI

SEQ ID NO: 2 - protein, 171 aa, synthetic construct
ALLEVVNDLFEEQTDLEKIVKKIMHRAQTLLKCERCSVLLLEDIESPVVKFTKSFELMSPKCSADAENSFKESVEKSSYSDWLINNSIAELVASTGLPVNVSDAYQDPRFDAEADQISGFHIRSVLCVPIWNSNHQIIGVAQVLNRLDGKPFDDADQRLFEAFVIFCGLGI

SEQ ID NO: 3 - DNA, 1242 bp, synthetic construct
  source 1..1242: mol_type = other DNA
ggatccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggccctgctggaggtggtcaatgacctctttgaagaacagactgatctggaaaagattgtcaagaaaataatgcatcgggcccaaactctgctgaaatgtgaacgctgttccgtcttacttctagaagacattgagtccccagtggtgaagtttaccaaatcctttgaactgatgtccccaaagtgcagcgctgatgctgagaacagtttcaaagagagtgtggagaaatcgtcttactctgactggctgataaacaacagcatcgctgagctggttgcttcgacaggccttcctgtgaatgtcagcgatgcctaccaggacccacgctttgacgctgaggctgaccagatatctggttttcatataagatctgttctctgtgtccccatttggaacagcaaccaccaaataattggggtggctcaagtgttgaacagactcgacggaaaaccttttgatgatgcagaccaaaggctttttgaggcctttgtcatcttttgtggccttggtatttaactcgag

SEQ ID NO: 4 - protein, 14 aa, synthetic construct
ALLEVVNDLFEEQT

SEQ ID NO: 5 - protein, 933 aa, synthetic construct
MAASRLDFGEVETFLDRHPELFEDYLMRKGKQELVDKWLQRHTSGQGASSLRPALAGASSLAQSNAKGSPGIGGGAGPQGSAHSHPTPGGGESAGVPLSPSWASGSRGDGSLQRRASQKELRKSFARSKAIHVNRTYDEQVTSRAQEPLSSVRRRALLRKASSLPPTTAHILSALLESRVNLPQYPPTAIDYKCHLKKHNERQFFLELVKDISNDLDLTSLSYKILIFVCLMVDADRCSLFLVEGAAAGKKTLVSKFFDVHAGTPLLPCSSTENSNEVQVPWGKGIIGYVGEHGETVNIPDAYQDRRFNDEIDKLTGYKTKSLLCMPIRNSDGEIIGVAQAINKVPEGAPFTEDDEKVMQMYLPFCGIAISNAQLFAASRKEYERSRALLEVVNDLFEEQTDLEKIVKKIMHRAQTLLKCERCSVLLLEDIESPVVKFTKSFELMSPKCSADAENSFKESVEKSSYSDWLINNSIAELVASTGLPVNVSDAYQDPRFDAEADQISGFHIRSVLCVPIWNSNHQIIGVAQVLNRLDGKPFDDADQRLFEAFVIFCGLGINNTIMYDQVKKSWAKQSVALDVLSYHATCSKAEVDKFKAANIPLVSELAIDDIHFDDFSLDVDAMITAALRMFMELGMVQKFKIDYETLCRWLLTVRKNYRMVLYHNWRHAFNVCQLMFAMLTTAGFQEILTEVEILAVIVGCLCHDLDHRGTNNAFQAKSDSALAQLYGTSATLEHHHFNHAVMILQSEGHNIFANLSSKEYSDLMQLLKQSILATDLTLYFERRTEFFELVRKGDYDWSITSHRDVFRSMLMTACDLGAVTKPWEISRQVAELVTSEFFEQGDRERSELKLTPSAIFDRNRKDELPRLQLEWIDSICMPLYQALVKVNAKLKPMLDSVAANRRKWEELHQKRLQVSAASPDPASPMVAGEDRL

(19) United States
(12) Patent Application Publication
(10) Pub. No.: US 2026/0317738 A1
(43) Pub. Date: Oct. 15, 2026

(54) INHIBITORS OF PDE11A4 AND METHODS OF USING SAME

(71) Applicant: UNIVERSITY OF MARYLAND, BALTIMORE, Baltimore, MD (US)

(72) Inventor: Michele Patrice Kelly, Fulton, MD (US)

(21) Appl. No.: 18/998,724

(22) PCT Filed: Jul. 28, 2023
(86) PCT No.: PCT/US2023/071206

§ 371 Date: Jan. 27, 2025

Related U.S. Application Data
(60) Provisional application No. 63/393,167, filed on Jul. 28, 2022.

(51) Int. Cl. C12N 9/16; A61K 38/46; A61P 25/28; C12N 5/071; C12N 5/079; C12N 5/16; C12N 15/85

(52) U.S. Cl. CPC C12N 9/16; A61K 38/465; A61P 25/28; C12N 5/0618; C12N 5/0682; C12N 5/0686; C12N 5/16; C12N 15/85; C12Y 301/04017; C12N 2510/04

(57) ABSTRACT

PDE11A4 inhibitors, and methods of using the same, are provided for treating or preventing diseases or disorder associated with cognitive decline.

### CROSS-REFERENCE TO RELATED APPLICATIONS

This application claims priority to and benefit of U.S. Provisional Patent Application No. 63/393,167, filed Jul. 28, 2022, which is incorporated by reference herein in its entirety.

### STATEMENT AS TO FEDERALLY SPONSORED RESEARCH

This invention was made with government support under Grant Numbers AG061200 and MH101130 awarded by the National Institutes of Health. The government has certain rights in the invention.

### SEQUENCE LISTING

The present application contains a Sequence Listing which has been submitted electronically in XML format and is hereby incorporated by reference in its entirety. Said XML copy, created on Jul. 28, 2023, is named “115834-5033-WO_SEQUENCE.xml” and is 12,288 bytes in size.

### FIELD

The disclosure relates generally to molecules, including polypeptides, that alter the subcellular location of PDE11A4, and methods of using such compounds as treatments for disease.

### BACKGROUND

After the age of 60, nearly all individuals experience some form of cognitive decline—particularly memory deficits—and no drugs are able to prevent or reverse this loss. Indeed, advanced age is the strongest risk factor for dementia. Even in absence of dementia, age-related cognitive impairment increases health care costs and risk for disability. Age-related cognitive decline is not a uniform process, with variability in symptom severity observed across individuals and across cognitive domains. Associative long-term memories (aLTMs)—particularly those involving experiences with family and friends—are more susceptible to age-related cognitive decline than are recognition long-term memories (rLTMs) for reasons that are not well understood. The lack of knowledge of the molecular mechanisms that govern age-related decline slows the development of novel therapeutics.

### SUMMARY

The present disclosure provides a novel class of inhibitors of PDE11A4 e.g., a useful therapeutic target for treating diseases and disorders including, but not limited to, social deficits associated with schizophrenia, bipolar disorder, or autism and cognitive deficits/dementia associated with age-related cognitive decline, traumatic brain injury, or Alzheimer's disease.

Accordingly, in exemplary embodiments, the disclosure provides an isolated fragment of PDE11A4 comprising a GAF-B binding sequence and comprising or consisting of a polypeptide sequence of SEQ ID NO: 1, or fragments thereof having from at least about 70% to at least about 99% identity thereto. In various embodiments, the disclosure provides an isolated fragment of PDE11A4 comprising a GAF-B binding sequence and comprising or consisting of a polypeptide sequence of SEQ ID NO: 2, or fragments thereof having from at least about 70% to at least about 99% identity thereto. SEQ ID NO: 2 is similar to SEQ ID NO: 1, but contains a 14 AA spacer (SEQ ID NO: 4) on its N-terminus.

In exemplary embodiments, the disclosure provides a polynucleotide encoding an isolated fragment of the invention. In some embodiments, the present disclosure provides vectors and host cells for preparing a polynucleotide of the invention using recombinant methods. In some embodiments, the host cell is a mammalian cell (e.g., CHO cell, a HEK-293 cell, or an Sp2.0 cell).

The present disclosure provides pharmaceutical compositions comprising an isolated fragment of the invention and a physiologically compatible carrier medium. In exemplary embodiments, a pharmaceutical composition provides a therapeutically effective amount for the treatment or prevention of a disease or disorder alleviated by inhibiting PDE11A4 activity e.g. in a patient in need thereof. In various embodiments, wherein the disease or disorder is associated with cognitive decline. In some embodiments, the disease or disorder is dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), and cognitive decline associate with spatial memory, other depression-related behaviors, additional anxiety-related behaviors, sensorimotor gating, or social behaviors.

The present disclosure provides methods of treating or preventing a disease or disorder alleviated by inhibiting PDE11A4 activity in a patient in need thereof. In exemplary embodiments, a method comprises administering to the patient a therapeutically effective amount of an isolated fragment of the invention. In various embodiments, a method comprises administering to the patient a therapeutically effective amount of a pharmaceutical composition the invention. In some embodiments, an isolated fragment of the invention is administered in a dosage unit form. In some embodiments, the dosage unit comprises a physiologically compatible carrier medium. In exemplary embodiments, a method treats or prevents a disease or disorder is associated with cognitive decline. In some embodiments, the disease or disorder is selected from dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), and cognitive decline associate with spatial memory, other depression-related behaviors, additional anxiety-related behaviors, sensorimotor gating, and social behaviors.

These and other embodiments, features, and potential advantages will become apparent with reference to the following description and drawings.

### BRIEF DESCRIPTION OF THE DRAWINGS

The foregoing summary, as well as the following detailed description of embodiments of the disclosure, are better understood when read in conjunction with the appended drawings and figures.

FIG. 1 is an image showing how PDE11A4 mRNA expression is restricted to the HIPP. Protein is expressed in neuronal cell bodies, dendrites, and axons.

FIGS. 2A-2C illustrate increases in hippocampal PDE11A expression. FIG. 2A is a graph of experimental data showing how hippocampal PDE11A expression is increased in old vs. young mice. FIG. 2B. is a graph of experimental data showing how hippocampal PDE11A expression is increased in adult (18-40 yrs) vs. prenatal humans. FIG. 2C is a graph of experimental data showing how hippocampal PDE11A expression is increased in demented vs. non-demented aged humans (>75 yrs) with a history of TBI. Post hoc*vs. Young, prenatal, or ‘No’ group, P<0.05. A.U.—arbitrary units.

FIGS. 3A-3C illustrate aging preferentially impairs aLTMs in mice. FIG. 3A is a graph of experimental data showing impaired aLTM for social transmission of food preference (STFP; n=25-34/group) 7 days after training. FIG. 3B is a graph of experimental data illustrating male and female aged C57BL/6J mice intact rLTM 7 days after training as measured by social odor recognition (SOR; n=15-16/age). FIG. 3C is a graph of experimental data illustrating non-social odor recognition (NSOR; n=7-8/age) in old and young mice. Memory refers to eating significantly more of the trained vs. novel food (FIG. 3A) or investigating the novel odor longer than the familiar odor (FIG. 3A, 3B). Young-2-6 months; Old-17-22 months. Post hoc*vs. familiar odor or novel food, P<0.01; ^{#}vs. Young, P=0.019.

FIG. 4A is a graph of experimental data showing male and female old PDE11A WT mice (WT-O) having no memory for STFP 7 days after training; however old PDE11A KO mice (KO-O) and heterozygous mice show robust memory equivalent to that of young (Y) mice. The protective effect of PDE11A deletion was replicated across sexes in 2 large cohorts, the combined analysis of which is shown here (n=26-29/group). The protective effect of PDE11A deletion on aLTMs is specific. FIG. 4B is a graph of experimental data showing there is no difference in rLTM between WT-O and KO-O mice, as measured in non-social odor recognition (NSOR; n=14). Young WTs were not included in this pilot study since aging does not impair NSOR—see FIG. 3.┘. Y=5-7 months; O=18-21 months. Post hoc*vs. novel food or familiar odor, P<0.001; ^{#}vs. WT-O, P<0.001.

FIG. 5A is an image of non-limiting example that due to the large size of PDE11A4 (>3 kb), a lentivirus was used to overexpress a GFP-mPDE11A4 fusion or GFP alone (negative control).

FIG. 5B shows western blots of DHIPP and VHIPP that illustrate titrating virus delivery allows overexpression of PDE11A4 in a dorsal<ventral gradient, as is seen in vivo.

FIG. 5C is a graph of experimental data showing how virally-expressed PDE11A4 engages relevant signal transduction cascades as it restores phosphorylation of the ribosomal protein S6 specifically at residues 235/236 (n=4-5/group), which was previously showed is significantly reduced in the hippocampus of KO vs WT.

FIG. 5D is a graph of experimental data illustrating mice trained on STFP and tested 7 days after training. KO mice expressing GFP in the hippocampus show strong aLTM for STFP; however, KO mice overexpressing PDE11A4 in the hippocampus (that is, mimicking the state of an “old WT”) fail to show significant aLTM for STFP.

FIG. 5E is a graph of experimental data illustrating the ability of PDE11A4 overexpression to impair aLTM, as KO mice treated with either the GFP or PDE11A4 lentivirus show strong rLTM for NSOR.

FIGS. 6A-6K illustrate that PDE11A homodimerization reverses molecular/biochemical phenotypes associated with aging independently of S162 phosphorylation. FIG. 6A is a graph of experimental data showing cGMP-PDE activity (n=5-6 biological replicates/group; effect of group: F(2,14)=168.01, P<0.001; Post hoc: GFP vs. all groups, P<0.001). FIG. 6B is a graph of experimental data showing cAMP-PDE activity (n=6 biological replicates/group; effect of group: F(2,14)=12.85, P<0.001; Post hoc: GFP vs. WC P<0.001, GFP vs. WG P=0.005) despite the fact that disrupting homodimerization did not directly affect PDE11A4 catalytic activity. FIG. 6C is a graph of experimental data showing attenuated PDE11A4-induced decreases in cGMP levels (n=14 biological replicates/group; effect of group: F(2,39)=4.33, P=0.020; Post hoc: GFP vs. WC P=0.020, WG vs. WC P=0.041). FIG. 6D is a graph of experimental data showing exacerbating PDE11A4-induced decreases in CAMP in COS1 cells (n=10 biological replicates/group; One Way ANOVA failed equal variance; ANOVA on Ranks for effect of group: H (2)=16.34, P<0.001; Post Hoc: GFP vs. all groups P<0.001, WC vs. WG P=0.034). FIG. 6E is a graph of experimental data demonstrating that disrupting homodimerization reduces back to WT levels the potentiated accumulation caused by the aging-related phosphomimic mutant S117D/S124D in HEK293T cells (n=12 biological replicates/group; effect of Pde11a4 plasmid: F(1, 44)=46.54, P<0.001; effect of Cherry plasmid: F(1,44)=10.31, P=0.002). FIG. 6F is a graph of experimental data demonstrating that disrupting homodimerization reduces back to WT levels the potentiated accumulation caused by the aging-related phosphomimic mutant S117D/S124D in HT-22 cells (n=12 biological replicates/group; effect of Pde11a4 plasmid: F(1, 44)=43.79, P<0.001; effect of Cherry plasmid: F(1,44)=23.08, P<0.001). FIG. 6G is a graph of experimental data demonstrating that S162D shifts PDE11A4 from the membrane to the cytosol relative to WT (n=7-8 biological replicates/group; effect of group: t(16)=−3.21, P=0.005). FIG. 6H is a graph of experimental data demonstrating that GAF-B is still able to reduce the accumulation of PDE11A4 even when a phosphomutant alanine is introduced at S162 (i.e., S162A) in HEK293T cells (n=12 biological replicates/group; effect of Cherry plasmid: F(1,26)=135.65, P<0.001). FIG. 6I is a graph of experimental data demonstrating that GAF-B is still able to reduce the accumulation of PDE11A4 even when a phosphomutant alanine is introduced at S162 (i.e., S162A) in COS1 cells (n=25-26 biological replicates/group; effect of Cherry plasmid: F(1,99)=137.52, P<0.001).

FIG. 6J is a graph of experimental data demonstrating that S162D differed substantially from GAF-B in terms of regulating cyclic nucleotide levels No effect on PDE11A4-induced decreases in cGMP levels (n=10-12 biological replicates/group; effect of group: F(2,30)=6.99, P=0.003; Post hoc GFP vs. WT P=0.004, GFP vs. 162D P=0.007, WT vs 162D P=0.924). FIG. 6K is a graph of experimental data demonstrating that attenuation of PDE11A4-induced decreases in cAMP levels (n=15 biological replicates/group; effect of group: F(2,42)=8.76, P<0.001; Post hoc GFP vs. WT P<0.001, WT vs. 162D P=0.020, GFP vs 162D P=0.089). * vs. GFP, WT, W-C, P<0.001; #vs. other mutant(s), P<0.001; Data plotted as individual data points and mean±SEM.

FIG. 7A is an image and graph of experimental data illustrating phosphomimic mutations of PDE11A4 at serines 117 and 124 (117D124D) synergize, increasing the accumulation of PDE11A4 in distinct structures. Phosphoresistant mutations S117AS124A have the opposite effect.

FIG. 7B is a graph and image showing how biochemical fractionation of S117DS124 shifts PDE11A4 from the cytosol to the membrane.

FIG. 7C is a graph of experimental data and image showing how S117 and S124 also synergize at the level of phosphorylation.

FIG. 7D is a graph of experimental data and image showing that disrupting PDE11A4 homodimerization using an isolated GAF-B (GB) domain decreases pS117 (P=0.07) and pS124.

FIG. 7E is a graph of experimental data normalizing the hyperaccumulation that is seen with S117D124D.

FIG. 7F is a graph of experimental data and image showing expression of S117D124D mimics age-related decreases in cGMP levels.

FIG. 7G is a graph of experimental data and image showing how expression of the isolated GAF-B domain has the opposite effect as what is shown in FIG. 7F.

FIG. 8. is an image showing immuno-fluorescence with a total PDE11A antibody (top-green) and pS117/pS124-PDE11A4 antibody (bottom-green) suggest that age-related increases in PDE11A expression occur in a compartment-specific manner. KOs virally overexpressing PDE11AWT show the same pattern as “old”.

FIG. 9 is a graph of experimental data and image showing age-related increases in VHIPP PDE11A4 protein expression occurs preferentially in membrane fractions, consistent with the in vitro studies showing S117D/S124D mutations shift PDE11A4 from the cytosol to the membrane.

FIG. 10 is an image showing how mPde11a4-mCherry reports transcriptional activity in ventral CA1 (vCA1) but not ventral dentate gyrus (vDG), consistent with endogenous expression pattern of mPde11A4 (see also FIG. 1). In contrast, the ubiquitous Pgk-mCherry construct reports transcription in both subregions.

FIG. 11 is a graph of experimental data showing how p54nrb/NONO and XRN2 mRNA expression are reduced with age in human hippocampus. These reductions significantly correlate with the age-related increases in PDE11A mRNA expression shown in FIG. 2B (p54nrb/NONO: r=−0.424, P=0.016; XRN2: r=−0.352, P=0.048).

FIG. 12A is an image showing widefield fluorescence's ability to label PDE11A4 mRNA (red) using RNAscope. A confocal microscope in the USC IRF are used to collect z-stack images.

FIG. 12B is an image showing an example of subcellular resolution provided by confocal microscope. Shown is PDE11A4 protein (green) in COS1 cells counterstained for the nuclear marker DAPI (blue). Optical sections through the cell (shown to the right and bottom) clarify whether labeling observed from above is in the nucleus or cytosol.

FIGS. 13A-13G illustrate that disruption PDE11A homodimerization in vivo selectively decreases PDE11A4 expression in a compartment-specific manner and is sufficient to decrease PDE11A4 accumulations in ghost axons that occur with age. FIG. 13A is an image of a lentiviral construct containing either mCherry (i.e., negative control) or an mCherry-tagged isolated GAF-B domain (GAF-B) was injected bilaterally into dorsal and ventral CA1 of hippocampus. FIG. 13B is an image showing that that injection of the GAF-B construct disrupted PDE11A4 homodimerization; whereas, mCherry alone did not. FIG. 13C is an image showing stereotaxic delivery of the lentiviral constructs (mCherry and mCherry-GAF-B) targeting dorsal (DHIPP) and ventral (VHIPP) CA1 sub-regions of the hippocampus resulted in high expression within CA1 in all subjects, with a subset of mice demonstrating a diffuse expression in dentate gyrus, CA2, and CA3. FIG. 13D is an image showing stereotaxic delivery of the lentiviral constructs (mCherry and mCherry-GAF-B) targeting dorsal (DHIPP) and ventral (VHIPP) CA1 sub-regions of the hippocampus resulted in high expression within CA1 in all subjects, with a subset of mice demonstrating a diffuse expression in dentate gyrus, CA2, and CA3. FIG. 13E is an image showing expression of mCherry-GAF-B decreased PDE11A4 expression in distal dendrites (dotted-arrows) and axons (solid arrows).

FIG. 13F is an image showing the GAF-B construct was also able to reduce age-related increases in so-called “PDE11A ghost axons” (i.e., filamentous structures where PDE11A4 accumulates with age; shown: “140/150” cocktail of antibodies). FIG. 13G is a graph of experimental data demonstrating that in CA1 of old Pde11a wild-type (WT) mice, two different antibody cocktails including PDE11A #1 (fails normality; Rank Sum Test for effect of group: T(6,16)=99.00, P=0.022) and PDE11A-140/150 (fails normality; Rank Sum Test for effect of group: T(7,15)=109.00, P=0.043), revealed a significant decrease in PDE11A4-filled structures in GAF-B-treated mice relative to mCherry-treated mice (GAF-B, n=7M,9F; mCherry, n=3M,3F). The 140/150 combination detects phosphorylation of serines 117 and 124—a key intramolecular mechanism that drives the accumulation of PDE11A4 in ghost axons. Brightness, histogram stretch, and/or contrast of images adjusted for graphical clarity.

FIGS. 14A-14G illustrate that disrupting PDE11A homodimerization in the hippocampus of old mice is sufficient to reverse age-related decline of remote long-term social memory. FIG. 14A is a graph of experimental data showing that using the social transmission of food preference assay (STFP), it was found that GB-O mice exhibited impaired recent LTM relative to mCh-O and young mice (GB-O; n=3M/3F, mCh-O; n=3M/2F, Young; n=5F; effect of group: F(2,13)=16.52, P<0.001; Post hoc: GAF-B vs each group, P<0.001). FIG. 14B is a graph of experimental data showing GB-O mice (n=3M/3F) and young mice (n=8F) demonstrated significant remote LTM for STFP, but mCh-O mice did not (n=4M/3F). To determine if disrupting homodimerization alters the non-social and/or social recognition components of the STFP assay, odor recognition memory was tested in these mice. FIG. 14C is a graph of experimental data showing GB-O and mCh-O mice learned equally well relative to young mice during non-social odor recognition (NSOR) training (GB-O, n=5M/10F; mCh-O, n=5M/11F; Young, n=2M/6F; effect of trial for novel odor: F(1,45)=76.58, P<0.001). FIG. 14D is a graph of experimental data showing all groups also demonstrated equivalent NSOR recent LTM (effect of group: F(2,45)=0.92, P=0.405) and E) NSOR remote LTM (effect of group: F(2,42)=0.53, P=0.590). FIG. 14F is a graph of experimental data showing GB-O and mCh-O mice learned equally well relative to young mice during social odor recognition training (SOR) (effect of trial for novel odor: F(1,46)=115.79, P<0.001). FIG. 14G is a graph of experimental data showing GB-O mice showed significantly improved remote SOR memory relative to mCh-O mice (effect of group: F(2,45)=3.76, P=0.031; Post hoc: mCh-O vs. GB-O P=0.029). #vs Young and mCh-O, P=0.031−0.001; \hat{ } vs Trial 1, P<0.001; * has memory (i.e., significantly >0), P=0.045 to <0.001 (see Table 1 for one-sample t-tests). Data plotted as individual points (females as circles, males as squares) and expressed as mean±SEM.

### DEFINITIONS

Unless defined otherwise, all technical and scientific terms used herein have the same meaning as is commonly understood by one of skill in the art to which this disclosure belongs. All patents and publications referred to herein are incorporated by reference in their entireties.

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The terms “active pharmaceutical ingredient” and “drug” include the polypeptides, polynucleotides, and compositions described herein. The terms “active pharmaceutical ingredient” and “drug” may also include those compounds described herein that bind PDE11A4 and thereby modulate (e.g. inhibit) PDE11A4 activity.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc., which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the terms “treat,” “treatment,” and/or “treating” may refer to the management of a disease, disorder, or pathological condition, or symptom thereof with the intent to cure, ameliorate, stabilize, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition). As used herein, the terms “prevent,” “preventing,” and/or “prevention” may refer to reducing the risk of developing a disease, disorder, or pathological condition.

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule. For example, the compounds described herein may modulate (e.g., inhibit) PDE11A4 protein. In some embodiments, the compounds described herein may selectively modulate (e.g., selectively inhibit) PDE11A4 protein as compared to other PDE11A proteins. In some embodiments, the compounds described herein may selectively modulate (e.g., selectively inhibit) PDE11A4 protein as compared to other PDE or PDE11A proteins. “Modulate” and “modulation” also include changing the subcellular localization and/or location of PDE11A4. “Modulate” and “modulation” also include disrupting and/or preventing homodimerization of PDE11A4. “Modulate” and “modulation” also include direct modulation of PDE11A4 (e.g. modulation of catalytic activity of PDE11A4). “Modulate” and “modulation” also include indirect modulation of PDE11A4 (e.g. disrupting and/or preventing homodimerization of PDE11A4). “Inhibit” and “inhibiting” also include changing the subcellular localization and/or location of PDE11A4. “Inhibit” and “inhibiting” also include disrupting and/or preventing homodimerization of PDE11A4. “Inhibit” and “inhibiting” also include direct inhibition of PDE11A4 (e.g. inhibition of catalytic activity of PDE11A4). “Inhibit” and “inhibiting” also include indirect inhibition of PDE11A4 (e.g. disrupting and/or preventing homodimerization of PDE11A4 and/or degrading PDE11A4).

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)). In some embodiments, the term “conservative sequence modifications” are used to refer to amino acid modifications that do not significantly affect or alter the binding characteristics of the antibody containing the amino acid sequence.

The terms “percent identical” or “percent identity,” in the context of two or more nucleic acids or polypeptide sequences, refers to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman, Adv. Appl. Math. 2: 482c (1970), by the homology alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., Current Protocols in Molecular Biology, 2003).

Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff, (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444-453, 1970) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Balzer et al., (1991) Nucleic Acid Res. 19:5081; Ohtsuka et al., (1985) J. Biol. Chem. 260:2605-2608; and Rossolini et al, (1994) Mol. Cell. Probes 8:91-98).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” or “physiologically compatible” carrier or carrier medium is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, or from 0% to 10%, or from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

### DETAILED DESCRIPTION

### Inhibition of PDE11A4

An enzyme called phosphodiesterase 11A (PDE11A) and its role in the neurobiological substrates of memory and social behaviors has been examined. PDE11A is a member of the large phosphodiesterase enzyme family and was originally cloned in 2000. The enzyme is derived from a single gene product, hydrolyzes both cAMP and cGMP and exists in 4 isoforms. The enzyme is predominantly expressed in the brain. In particular, PDE11A is found in the anterior hippocampus and more specifically in neurons in the superficial layer of CA1, the subiculum and the amygdalohippocampal area of the hippocampus. Moreover, PDE11A4 is the only PDE whose expression in brain emanates from the hippocampus, a region of the brain associated with associated long term memory (aLTM). Little is known of the signaling pathways lying up or downstream of PDE11A4, but it has been shown that PDE11A appears to regulate important signals for memory consolidation, including glutamatergic and calcium/calmodulin-dependent kinase II (CamKII) signaling, as well as protein synthesis. It has been shown that CAMP and cGMP signaling are decreased in aged and demented hippocampus in rats and humans. These age-related decreases in cyclic nucleotides are associated with increased expression of PDE11A4 in rodents and in humans with hippocampal dementia, versus non-demented aged subjects with a history of traumatic brain injury.

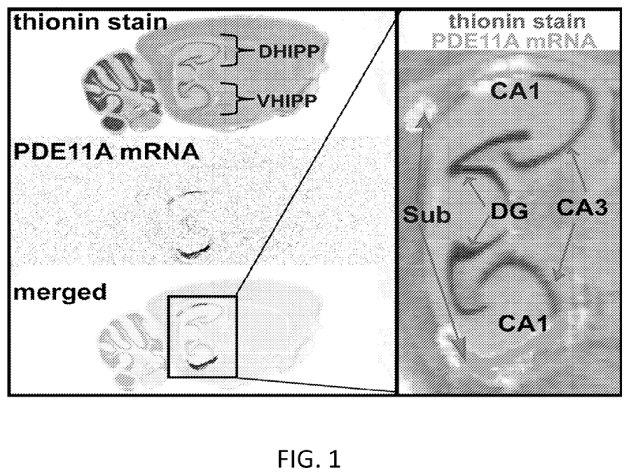

The PDE11A family, which breaks down cAMP and cGMP, is comprised of a single gene that is spliced into 4 isoforms, PDE11A1 through PDE11A4. The longest isoform, PDE11A4, is the isoform that is expressed in brain and it is ~95% homologous across mouse, rat and human. This high degree of homology argues that the results obtained in rodent models translates across species. PDE11A single nucleotide polymorphisms (SNPs) have been associated with major depressive disorder (MDD), suicide risk, antidepressant response in patients with MDD, and lithium response in patients with bipolar disorder. Both MDD and BPD have been conceptualized as diseases of accelerated aging. It was established that PDE11A4 is expressed in the brain. PDE11A4 was found in brain because PDE11A knockout (KO) mice was phenotyped and what few phenotypes they had were found to be of relevance to ventral hippocampal function. Thus, the search for a PDE11A isoform in brain was directed to the ventral hippocampal formation (VHIPP) to detect expression restricted to this small brain region. Indeed, it was discovered that PDE11A4 was strongly expressed in neurons of the superficial layer of CA1, the subiculum, and the adjacently connected amygdalohippocampal area (AHi) of the VHIPP (FIG. 1A), with little expression in dorsal HIPP (DHIPP) and no expression in other brain regions or over 20 peripheral organs. Only the nervous system shows a specific PDE11A4 signal. This makes PDE11A4 very unique because in brain it is the only PDE to emanate only from the HIPP, a structure critical for social aLTMs. This enrichment of PDE11A4 in the HIPP has now been independently confirmed by multiple investigators. This—along with the fact that PDE11A is a highly druggable enzyme—makes PDE11A a very attractive drug target because it stands to selectively restore aberrant cyclic nucleotide signaling in a brain region affected by age-related decline without directly affecting signaling in other brain regions or peripheral organs that might lead to unwanted side effects. At the very least, PDE11A4 molecularly defines an exceptionally discrete circuit within a brain region key to learning and memory, making it ripe for the study of age-related cognitive decline.

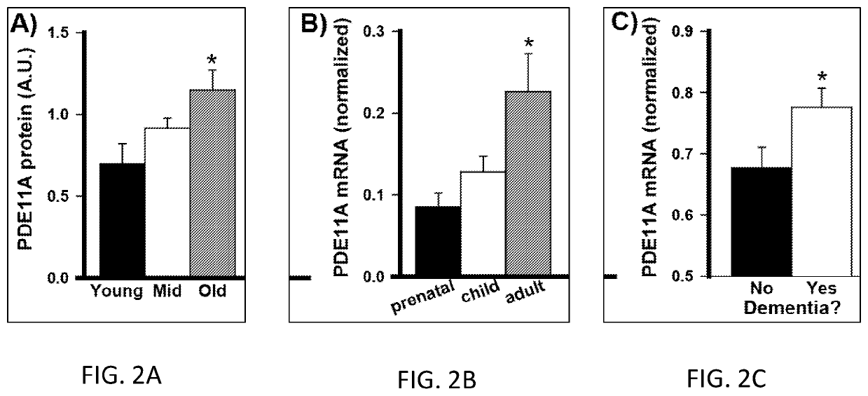

While not being bound by any particular theory, it is hypothesized that CAMP and cGMP signaling are decreased in the aged and demented hippocampus (rodents and humans), particularly when there is a history of traumatic brain injury (TBI). These age-related decreases in cyclic nucleotides are consistent with the observations that PDE11A4 expression increases with age in the rodent and human hippocampus and is significantly elevated in hippocampus of demented vs. non-demented aged humans with a history of TBI (FIG. 2).

Consistent with PDE11A4's restricted expression pattern, PDE11A KO mice appear normal on a wide range of sensory, motor and anxiety/depression-related behaviors, and show no gross peripheral pathology at least up to 1 year of age (later ages not assessed). Instead, PDE11A KO mice exhibit select social phenotypes such as preferring to interact with other PDE11A KO mice vs wild-type (WT) mice and showing differences in the consolidation of social memories. PDE11A KO mice also have an increased sensitivity to the behavioral effects of lithium. Little has been characterized of the signaling pathways lying up or downstream of PDE11A4, but it was shown that PDE11A appears to regulate signals that are important for memory consolidation, including glutamatergic and calcium/calmodulin-dependent kinase II (CamKII) signaling as well as protein synthesis.

PDEs are discretely localized to specific subcellular domains. As a result, PDEs do not simply control the total cellular content of cyclic nucleotides, they generate individual pools or nanodomains of cyclic nucleotide signaling. Such subcellular compartmentalization of cyclic nucleotide signaling allows a single cell to respond discretely to diverse intra- and extracellular signals. Thus, where a PDE is localized is just as important to its overall function as is its catalytic activity. While some cyclases and PDEs responsible for generating and breaking down cAMP/cGMP are expressed more in the cytosol than the membrane (like PDE11A4), others are enriched in the membrane (like the closely related PDE2A and PDE10A). Interestingly, cyclic nucleotide signaling deficits observed in bipolar disorder and Alzheimer's disease appear to be more prominent in the cytosolic as opposed to membrane fractions.

Associative long-term memories (aLTMs)—particularly those involving friends and family—are more susceptible to age-related cognitive decline than are recognition long-term memories (rLTMs) for reasons that are not well understood. The lack of knowledge of the molecular mechanisms that govern age-related decline slows the development of novel therapeutics. Age-related increases in phosphodiesterase 11A (PDE11A), an enzyme that breaks down cAMP/cGMP and regulates social behaviors, may be a fundamental mechanism underlying age-related cognitive decline of aLTMs for social experiences. With regard to its tissue expression profile, the best controlled studies to date suggest that the longest isoform PDE11A4 is almost exclusively expressed in the ventral hippocampal formation (a.k.a. anterior HIPP in primates), specifically within neurons of the subiculum, superficial layer of CA1, and the adjacently connected amygdalohippocampal area. This makes PDE11A4 the ONLY PDE to be preferentially expressed in the HIPP, a brain region key to social aLTMs. Previous studies suggest that CAMP and cGMP signaling are decreased in the aging and demented HIPP (rodents and humans), consistent with the novel observations of aging and dementia-related increases in HIPP PDE11A4 expression in rodents and humans.

Decreases in 3′,5′-cyclic nucleotide signaling in the aged and demented hippocampus contribute to cognitive decline and are related, in part, to changes in the enzymes that break down cyclic nucleotides. There are eleven families of 3′,5′-cyclic nucleotide phosphodiesterases (PDEs) that are the only known enzymes to hydrolyze 3′,5′-cyclic adenosine monophosphate (cAMP) and 3′,5′-cyclic guanosine monophosphoate (cGMP), and their expression across subcellular compartments differs between families. For example, some PDEs are expressed more in the cytosol than the membrane (e.g., PDE11A), while others are more highly expressed in the membrane versus cytosol (including PDE2A, PDE9A and PDE10A. Due to the fact that PDEs are localized to specific subcellular domains, they are able to regulate individual pools or nanodomains of cyclic nucleotide signaling. Such subcellular compartmentalization of cyclic nucleotide signaling allows a single cell to respond specifically to simultaneous intra- and/or extracellular signals. Therefore, the subcellular localization of any PDE is equally important to its actual catalytic activity when considering its function. PDEs can become overexpressed and/or mislocalized with age and/or disease, which compromises the integrity of this physiological segregation of signals. Indeed, age-related diseases and neuropsychiatric diseases can show a loss of cyclic nucleotide signaling in one subcellular compartment but not another, suggesting therapeutic strategies should optimally target enzymes in a compartment-specific manner.

Of the eleven families of PDEs, phosphodiesterase 11A (PDE11A) has garnered particular interest in the context of altered cyclic nucleotide signaling related to ARCD and early-onset Alzheimer's disease. PDE11A is encoded by a single gene and has four isoforms. While protein for PDE11A4—the isoform expressed in brain—is found across all subcellular compartments, it is particularly enriched in the cytosolic versus membrane and nuclear compartments. The PDE11A catalytic domain is located within the C-terminal region, which is common to all isoforms, while the N-terminal region serves a regulatory function and is unique to each isoform. The regulatory N-terminus of PDE11A4, the longest PDE11A isoform, is unique in that it contains two full GAF (cGMP binding PDE, Anabaena adenylyl cyclase and E. coli FhlA) domains. The GAF-A domain binds cGMP as a potential allosteric regulatory site and the GAF-B domain regulates protein-protein interactions, including homodimerization. PDE11A4 is unique in that it is the only PDE whose expression in brain emanates solely from the extended hippocampal formation, a brain region critical to learning and memory and vulnerable to age-related deficits in cyclic nucleotide signaling. Possibly contributing to these hippocampal cyclic nucleotide signaling deficits are age-related increases in PDE11A4 expression that are conserved across mice, rats and humans. These age-related increases in PDE11A4 protein expression are deleterious as 1) Pde11a KO mice are protected against age-related cognitive decline (ARCD) of remote social associative long-term memories (aLTMs) and 2) mimicking age-related overexpression of PDE11A4 in the CA1 field of hippocampus of either young or old Pde11a KO mice is sufficient to mimic ARCD of remote social aLTMs.

While PDE11A4 protein expression in the young adult hippocampus is significantly higher in the cytosolic versus membrane compartment, age-related increases in PDE11A4 are found specifically in the membrane compartment of the ventral hippocampal formation (VHIPP). Therefore, these age-related increases in membrane-associated PDE11A4 may be considered ectopic. That said, the membrane pool of PDE11A4—although lesser in relative quantity—continually shows itself to be critical in regulating social behaviors. For instance, it was found that social isolation selectively decreases expression of membrane-associated PDE11A4 in the VHIPP, and that these isolation-induced decreases in PDE11A4 are sufficient to cause changes in subsequent social preferences and social memory. Similarly, PDE11A4 protein expression differences in VHIPP of BALB/cJ versus C57BL/6J mice are restricted to the membrane pool. The increased protein expression of membrane-associated PDE11A4 found in the VHIPP of BALB/cJ vs C57BL/6J mice appears to be driven by a single point mutation at amino acid 499 within the GAF-B domain, which strengthens the homodimerization and punctate accumulation of PDE11A4. Interestingly, disrupting PDE11A4 homodimerization in vitro by expressing an isolated GAF-B domain that acts as a negative sink disperses the punctate accumulation of PDE11A4 and selectively reduces expression of membrane-associated PDE11A4. This suggests that disrupting PDE11A4 homodimerization in vivo may represent a therapeutic option capable of treating age-related increases in PDE11A4 expression in a compartment-specific manner and, thus, ARCD of social memories. Indeed, it was previously showed that social preference of C57BL/6J mice can be altered by manipulating PDE11A4 homodimerization selectively within the CA1 field of hippocampus.

In one aspect, the disclosure provides a novel class of inhibitors of PDE11A4. In embodiments, inhibitors of PDE11A4 include molecules (e.g. polypeptides) capable of changing the subcellular localization and/or location of PDE11A4, and/or disrupting and/or preventing homodimerization of PDE11A4, and/or degrading PDE11A4. In embodiments, PDE11A4 is a useful therapeutic target for treating social deficits associated with schizophrenia, bipolar disorder, or autism as well as for treating cognitive deficits/dementia associated with age-related cognitive decline, traumatic brain injury, or Alzheimer's disease.

In one aspect, the disclosure provides an isolated fragment of PDE11A4, which comprises an isolated GAF-B domain of PDE11A4 that disrupts homodimerization and is capable of 1) altering social preferences/compatibility within the context of neuropsychiatric or neurodevelopmental disorders, 2) reversing cognitive decline associated with aging, dementia associated with traumatic brain injury, and/or Alzheimer's disease, and/or 3) alleviating other disorders where PDE11A4 forms accumulated proteinopathies, particularly in the membrane fraction. In a non-limiting embodiments, the role of PDE11A4 in actual brain function is explored, and intramolecular mechanisms that control how PDE11A4 functions in terms of enzymatic activity and subcellular trafficking are examined.

### PDE11A4 Inhibitors and Methods of Modulating PDE11A4

In an embodiment, the disclosure includes molecules capable of inhibiting PDE11A4, including but not limited to disrupting and/or preventing homodimerization of PDE11A4, and including but not limited to isolated fragments of PDE11A4 comprising a GAF-B binding sequence. In some embodiments, the PDE11A4 inhibitor is a molecule (e.g. polypeptide) capable of changing the subcellular localization and/or location of PDE11A4. In some embodiments, the PDE11A4 inhibitor is a molecule (e.g. polypeptide) capable of disrupting and/or preventing homodimerization of PDE11A4. In some embodiments, the PDE11A4 inhibitor is a molecule (e.g. polypeptide) capable of degrading PDE11A4 (e.g. a PDE11A4 degrader). In a non-limiting example, the PDE11A4 degraders of the disclosure disrupt and/or prevent homodimerization of PDE11A4, thereby changing the subcellular localization and/or location of PDE11A4 and triggering degradation of PDE11A4. In one embodiment, the PDE11A4 inhibitor is a PDE11A4 selective inhibitor. In an embodiment, the PDE11A4 inhibitor of the disclosure is about 1-fold, about 5-fold, about 10-fold, about 20-fold, about 50-fold, about 100-fold, about 500-fold, or about 1000-fold more selective for PDE11A4 over PDE11A1, PDE11A2, and/or PDE11A3.

In one aspect, the disclosure provides an isolated fragment of PDE11A4 comprising a GAF-B binding sequence. See for example Smith et al., Mol. Psychiatry 26:7107-7117 (2011), which is incorporated by reference herein in its entirety. In some embodiments, the isolated fragment comprises or consists of a polypeptide sequence of SEQ ID NO: 1, or a sequence having at least about 70%, at least about 75%, at least about 80%, at least about 85%, at least about 87%, at least about 89%, at least about 90%, at least about 91%, at least about 92%, at least about 93%, at least about 94%, at least about 95%, at least about 96%, at least about 97%, at least about 98%, or at least about 99% identity thereto. In some embodiments, the isolated fragment comprises or consists of a polypeptide sequence of SEQ ID NO: 2, or a sequence having at least about 70%, at least about 75%, at least about 80%, at least about 85%, at least about 87%, at least about 89%, at least about 90%, at least about 91%, at least about 92%, at least about 93%, at least about 94%, at least about 95%, at least about 96%, at least about 97%, at least about 98%, or at least about 99% identity thereto.

| SEQ ID | | |
|---|---|---|
| NO. | Description | Sequence |
| 1 | aa402-558 of Pdella4 | DLEKIVKKIMHRAQTLLKCERCSVLLLEDIESPVVKFTKSFELM |
| | GAF-B domain | SPKCSADAENSFKESVEKSSYSDWLINNSIAELVASTGLPVNVS |
| | | DAYQDPRFDAEADQISGFHIRSVLCVPIWNSNHQIIGVAQV |
| | | LNRLDGKPFDDADQRLFEAFVIFCGLGI |
| 2 | aa388-558 of Pdella4 | ALLEVVNDLFEEQTDLEKIVKKIMHRAQTLLKCERCSVLLLEDI |
| | GAF-B domain (spacer | ESPVVKFTKSFELMSPKCSADAENSFKESVEKSSYSDWLINNSI |
| | underlined) | AELVASTGLPVNVSDAYCDPRFDAEADQISGFHIRSVLCVPIWN |
| | | SNHQIIGVAQVLNRLDGKPFDDADQRLFEAFVIFCGLGI |
| 3 | Nucleic acid encoding | GGATCCGCCACCATGGTGAGCAAGGGCGAGGAGGATAACATGGC |
| | amino acids 388-558 of | CATCATCAAGGAGTTCATGCGCTTCAAGGTGCACATGGAGGGCT |
| | mouse pdella | CCGTGAACGGCCACGAGTTCGAGATCGAGGGCGAGGGCGAGGGC |
| | (NP_001074502.1) | CGCCCCTACGAGGGCACCCAGACCGCCAAGCTGAAGGTGACCAA |
| | | GGGTGGCCCCCTGCCCTTCGCCTGGGACATCCTGTCCCCTCAGT |
| | | TCATGTACGGCTCCAAGGCCTACGTGAAGCACCCCGCCGACATC |
| | | CCCGACTACTTGAAGCTGTCCTTCCCCGAGGGCTTCAAGTGGGA |
| | | GCGCGTGATGAACTTCGAGGACGGCGGCGTGGTGACCGTGACCC |
| | | AGGACTCCTCCCTGCAGGACGGCGAGTTCATCTACAAGGTGAAG |
| | | CTGCGCGGCACCAACTTCCCCTCCGACGGCCCCGTAATGCAGAA |
| | | GAAGACCATGGGCTGGGAGGCCTCCTCCGAGCGGATGTACCCCG |
| | | AGGACGGCGCCCTGAAGGGCGAGATCAAGCAGAGGCTGAAGCTG |
| | | AAGGACGGCGGCCACTACGACGCTGAGGTCAAGACCACCTACAA |
| | | GGCCAAGAAGCCCGTGCAGCTGCCCGGCGCCTACAACGTCAACA |
| | | TCAAGTTGGACATCACCTCCCACAACGAGGACTACACCATCGTG |
| | | GAACAGTACGAACGCGCCGAGGGCCGCCACTCCACCGGCGGCAT |
| | | GGACGAGCTGTACAAGGCCCTGCTGGAGGTGGTCAATGACCTCT |
| | | TTGAAGAACAGACTGATCTGGAAAAGATTGTCAAGAAAATAATG |
| | | CATCGGGCCCAAACTCTGCTGAAATGTGAACGCTGTTCCGTCTT |
| | | ACTTCTAGAAGACATTGAGTCCCCAGTGGTGAAGTTTACCAAAT |
| | | CCTTTGAACTGATGTCCCCAAAGTGCAGCGCTGATGCTGAGAAC |
| | | AGTTTCAAAGAGAGTGTGGAGAAATCGTCTTACTCTGACTGGCT |
| | | GATAAACAACAGCATCGCTGAGCTGGTTGCTTCGACAGGCCTTC |
| | | CTGTGAATGTCAGCGATGCCTACCAGGACCCACGCTTTGACGCT |
| | | GAGGCTGACCAGATATCTGGTTTTCATATAAGATCTGTTCTCTG |
| | | TGTCCCCATTTGGAACAGCAACCACCAAATAATTGGGGTGGCTC |
| | | AAGTGTTGAACAGACTCGACGGAAAACCTTTTGATGATGCAGAC |
| | | CAAAGGCTTTTTGAGGCCTTTGTCATCTTTTGTGGCCTTGGTAT |
| | | TTAACTCGAG |
| 4 | Spacer | ALLEVVNDLFEEQT |

In some embodiments, the isolated fragment of PDE11A4 comprising a GAF-B binding sequence further comprises a fusion protein. In some embodiments, the fusion protein comprises the isolated fragment and a moiety selected from the group consisting of an immunoglobulin fragment (e.g., an immunoglobulin Fc domain), serum albumin (e.g., human serum albumin), transferrin, and Fn3, or variants thereof. In certain embodiments of the foregoing aspects, the isolated fragment of PDE11A4 comprising a GAF-B binding sequence comprises the isolated fragment conjugated to a non-protein polymer, such as polyethylene glycol. In some embodiments, the isolated fragment of PDE11A4 comprising a GAF-B binding sequence comprises the isolated fragment operably linked to an immunoglobulin Fc domain. In some embodiments, the isolated fragment of PDE11A4 comprising a GAF-B binding sequence comprises the isolated fragment operably linked to human serum albumin.

### Nucleic Acids, Vectors and Host Cells

In aspects, the disclosure also provides polynucleotides encoding any of the polypeptides, including SEQ ID NO: 1 or 2, described herein. In aspects, the disclosure also provides a method of making any of the polynucleotides described herein. Polynucleotides can be made and expressed by procedures known in the art.

The sequence of a desired polypeptide and/or fragment thereof, and nucleic acid encoding such antibody, or fragment thereof, can be determined using standard sequencing techniques. A nucleic acid sequence encoding a desired polypeptide and/or fragment thereof, may be inserted into various vectors (such as cloning and expression vectors) for recombinant production and characterization.

In one aspect, the disclosure provides polynucleotides encoding the polypeptide sequence of SEQ ID NO: 1. In some embodiments, the polynucleotide encoding the SEQ ID NO: 1 encodes an amino acid sequence having at least about 70%, at least about 75%, at least about 80%, at least about 85%, at least about 87%, at least about 89%, at least about 90%, at least about 91%, at least about 92%, at least about 93%, at least about 94%, at least about 95%, at least about 96%, at least about 97%, at least about 98%, or at least about 99% identity thereto.

In one aspect, the disclosure provides polynucleotides encoding the polypeptide sequence of SEQ ID NO: 2. In some embodiments, the polynucleotide encoding the SEQ ID NO: 2 encodes an amino acid sequence having at least about 70%, at least about 75%, at least about 80%, at least about 85%, at least about 87%, at least about 89%, at least about 90%, at least about 91%, at least about 92%, at least about 93%, at least about 94%, at least about 95%, at least about 96%, at least about 97%, at least about 98%, or at least about 99% identity thereto.

In one aspect, the disclosure provides the polynucleotide sequence of SEQ ID NO: 3, or a polynucleotide sequence having at least about 70%, at least about 75%, at least about 80%, at least about 85%, at least about 87%, at least about 89%, at least about 90%, at least about 91%, at least about 92%, at least about 93%, at least about 94%, at least about 95%, at least about 96%, at least about 97%, at least about 98%, or at least about 99% identity thereto.

In some embodiments, SEQ ID NO: 1, SEQ ID NO: 2, and/or SEQ ID NO: 3 further comprise a tag (e.g. mCherry).

In some embodiments, the disclosure provides a polynucleotide sequence comprising amino acids 561-567 of PDE11A and/or further comprising a tag (e.g. mCherry). (see SEQ ID NO: 5 for reference):

| (SEQ ID NO: 5) | |
|---|---|
| 1 | maasrldfge vetfldrhpe lfedylmrkg kqelvdkwlq rhtsgqgass lrpalagass |
| 61 | laqsnakgsp gigggagpqg sahshptpgg gesagvplsp swasgsrgdg slqrrasqke |
| 121 | lrksfarska ihvnrtydeq vtsraqepls svrrrallrk asslppttah ilsallesrv |
| 181 | nlpqypptai dykchlkkhn erqfflelvk disndldlts lsykilifvc lmvdadrcsl |
| 241 | flvegaaagk ktlvskffdv hagepllpcs stensnevqv pwgkgiigyv gehgetvnip |
| 301 | dayqdrrfnd eidkltgykt ksllcmpirn sdgeiigvaq ainkvpegap fteddekvmq |
| 361 | mylpfcgiai snaqlfaasr keyersrall evvndlfeeq tdlekivkki mhraqtllkc |
| 421 | ercsvllled iespvvkftk sfelmspkcs adaensfkes vekssysdwl innsiaelva |
| 481 | stglpvnvsd ayqdprfdae adqisgfhir svlcvpiwns nhqiigvaqv lnrldgkpfd |
| 541 | dadqrlfeaf vifcglginn timydqvkks wakqsvaldv lsyhatcska evdkfkaani |
| 601 | plvselaidd ihfddfsldv damitaalrm fmelgmvqkf kidyetlerw lltvrknyrm |
| 661 | vlyhnwrhaf nvcqlmfaml ttagfqeilt eveilavivg clchdldhrg tnnafqaksd |
| 721 | salaqlygts atlehhhfnh avmilqsegh nifanlsske ysdlmqllkq silatdltly |
| 781 | ferrteffel vrkgdydwsi tshrdvfrsm lmtacdlgav tkpweisrqv aelvtseffe |
| 841 | qgdrerselk ltpsaifdrn rkdelprlql ewidsicmpl yqalvkvnak lkpmldsvaa |
| 901 | nrrkweelhq krlqvsaasp dpaspmvage drl. |

The polynucleotides of this disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al., eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, for example.

### Vectors

In some embodiments, a vector is selected that is optimized for expression of polypeptides in CHO or CHO-derived cells, or in NSO cells. Exemplary vectors are described, e.g., in Running Deer et al., Biotechnol. Prog. 20:880-889 (2004).

Suitable cloning and expression vectors can include a variety of components, such as promoter, enhancer, and other transcriptional regulatory sequences. The vector may also be constructed to allow for subsequent cloning of polypeptide into different vectors. Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColEl, pCR1, RP4, phage DNAs, and shuttle vectors such as pSA3 and pAT28. These and many other cloning vectors are available from commercial vendors such as BioRad, Stratagene, and Invitrogen. Expression vectors are further provided. Expression vectors generally are replicable polynucleotide constructs that contain a polynucleotide according to the disclosure. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest and/or the polynucleotides themselves, can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

### Host Cells

The polypeptides and/or fragment thereof, may be made recombinantly using a suitable host cell. A nucleic acid encoding the polypeptides and/or fragment thereof can be cloned into an expression vector, which can then be introduced into a host cell, such as E. coli cell, a yeast cell, an insect cell, a simian COS cell, a Chinese hamster ovary (CHO) cell, or a myeloma cell where the cell does not otherwise produce an immunoglobulin protein, to obtain the synthesis of polypeptides and/or fragment thereof in the recombinant host cell. Preferred host cells include a HT-22 cell, a CHO cell, a Human embryonic kidney HEK-293 cell, or an Sp2.0 cell, among many cells well-known in the art. An antibody fragment can be produced by proteolytic or other degradation of a full-length nucleic acid, by recombinant methods, or by chemical synthesis. A polypeptide fragment, especially shorter polypeptides up to about 50 amino acids, can be conveniently made by chemical synthesis. Methods of chemical synthesis for proteins and peptides are known in the art and are commercially available.

In various embodiments, polypeptides and/or fragment thereof may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lecl3 CHO cells, and FUT8 CHO cells; PER.C6R cells (Crucell); and NSO cells. In some embodiments, polypeptides and/or fragment thereof, may be expressed in yeast. See, e.g., U.S. Publication No. US 2006/0270045 A1. In some embodiments, a particular eukaryotic host cell is selected based on its ability to make desired post-translational modifications to the anti-CDCP1 heavy chains and/or anti-CDCP1 light chains. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Non-limiting exemplary methods are described, e.g., in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

Isolated fragments (e.g. polypeptides) may be purified by any suitable method. Many methods of purifying polypeptides are known in the art. In some embodiments, polypeptides and/or fragment thereof are produced in a cell-free system. Non-limiting exemplary cell-free systems are described, e.g., in Sitaraman et al., Methods Mol. Biol. 498:229-44 (2009); Spirin, Trends Biotechnol. 22:538-45 (2004); Endo et al., Biotechnol. Adv. 21:695-713 (2003).

### Methods of Treatment

The compounds and compositions described herein can be used in methods for treating diseases and/or disorders. In some embodiments, the compounds and compositions described herein can be used in methods for treating diseases associated with PDE11A4 activity.

In one embodiment, the disclosure relates to a method of treating and/or preventing a disease or disorder alleviated by inhibiting PDE11A4 activity in a patient in need thereof, including administering to the patient a therapeutically effective amount of a PDE11A4 inhibitor. In some embodiments, the therapeutically effective amount of the PDE11A4 inhibitor administered to the patient is capable of changing the subcellular localization and/or location of PDE11A4. In some embodiments, the therapeutically effective amount of the PDE11A4 inhibitor administered to the patient is capable of disrupting and/or preventing homodimerization of PDE11A4. In some embodiments, the therapeutically effective amount of the PDE11A4 inhibitor administered to the patient is capable of degrading PDE11A4. In a non-limiting example, In some embodiments, the therapeutically effective amount of the PDE11A4 inhibitor administered to the patient is capable of disrupting and/or preventing homodimerization of PDE11A4, thereby changing the subcellular localization and/or location of PDE11A4 and triggering degradation of PDE11A4. In one embodiment, the PDE11A4 inhibitor comprises or consists of an isolated fragment of PDE11A4 comprising a GAF-B binding sequence. In some embodiments, the PDE11A4 inhibitor comprises or consists of an isolated fragment of PDE11A4 comprising a GAF-B binding sequence and comprising or consisting of a polypeptide sequence of SEQ ID NO: 1, or a sequence having at least about 70%, at least about 75%, at least about 80%, at least about 85%, at least about 87%, at least about 89%, at least about 90%, at least about 91%, at least about 92%, at least about 93%, at least about 94%, at least about 95%, at least about 96%, at least about 97%, at least about 98%, or at least about 99% identity thereto. In some embodiments, the PDE11A4 inhibitor comprises or consists of an isolated fragment of PDE11A4 comprising a GAF-B binding sequence and comprising or consisting of a polypeptide sequence of SEQ ID NO: 2, or a sequence having at least about 70%, at least about 75%, at least about 80%, at least about 85%, at least about 87%, at least about 89%, at least about 90%, at least about 91%, at least about 92%, at least about 93%, at least about 94%, at least about 95%, at least about 96%, at least about 97%, at least about 98%, or at least about 99% identity thereto. In some embodiments, the disease or disorder is associated with cognitive decline. In some embodiments, the disease or disorder is selected from dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), and cognitive decline associate with spatial memory, other depression-related behaviors, additional anxiety-related behaviors, sensorimotor gating, and social behaviors.

In some embodiments, “cognitive decline” includes be any negative change in an animal's cognitive function. For example cognitive decline, includes but is not limited to, memory loss (e.g. behavioral memory loss), failure to acquire new memories, confusion, impaired judgment, personality changes, disorientation, or any combination thereof.

### Pharmaceutical Compositions

In an embodiment, an active pharmaceutical ingredient or combination of active pharmaceutical ingredients, such as any of the PDE11A4 inhibitors of the disclosure (e.g. molecules (e.g. polypeptides) capable of changing the subcellular localization and/or location of PDE11A4, and/or disrupting and/or preventing homodimerization of PDE11A4, and/or degrading PDE11A4), is provided as a pharmaceutically acceptable composition. In some embodiments, the PDE11A4 inhibitor comprises or consists of an isolated fragment of PDE11A4 comprising a GAF-B binding sequence (e.g. SEQ ID NO: 1 or SEQ ID NO: 2).

In one embodiment, the disclosure relates to a pharmaceutical composition including a therapeutically effective amount of a PDE11A4 inhibitor for the treatment of a disease alleviated by inhibiting PDE11A4 activity (e.g. changing the subcellular localization and/or location of PDE11A4, and/or disrupting and/or preventing homodimerization of PDE11A4, and/or degrading PDE11A4) in a patient in need thereof, and a physiologically compatible carrier medium. In some embodiments, the disease is associated with cognitive decline. In some embodiments, the PDE11A4 inhibitor comprises or consists of an isolated fragment of PDE11A4 comprising a GAF-B binding sequence (e.g. SEQ ID NO: 1 or SEQ ID NO: 2). In some embodiments, the disease or disorder is selected from dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), and cognitive decline associate with spatial memory, other depression-related behaviors, additional anxiety-related behaviors, sensorimotor gating, and social behaviors.

In some embodiments, the concentration of each of the active pharmaceutical ingredients provided in the pharmaceutical compositions of the disclosure, such as any of the PDE11A4 inhibitors of the disclosure, for example an isolated fragment of PDE11A4 comprising a GAF-B binding sequence (e.g. SEQ ID NO: 1 or SEQ ID NO: 2), is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of each of the active pharmaceutical ingredients provided in the pharmaceutical compositions of the disclosure, such as any of the PDE11A4 inhibitors of the disclosure, for example an isolated fragment of PDE11A4 comprising a GAF-B binding sequence (e.g. SEQ ID NO: 1 or SEQ ID NO: 2), is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of each of the active pharmaceutical ingredients provided in the pharmaceutical compositions of the disclosure, such as any of the PDE11A4 inhibitors of the disclosure, for example an isolated fragment of PDE11A4 comprising a GAF-B binding sequence (e.g. SEQ ID NO: 1 or SEQ ID NO: 2), is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v, or v/v of the pharmaceutical composition.

In some embodiments, the concentration of each of the active pharmaceutical ingredients provided in the pharmaceutical compositions of the disclosure, such as any of the PDE11A4 inhibitors of the disclosure, for example an isolated fragment of PDE11A4 comprising a GAF-B binding sequence (e.g. SEQ ID NO: 1 or SEQ ID NO: 2), is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v, or v/v of the pharmaceutical composition.

In some embodiments, the amount of each of the active pharmaceutical ingredients provided in the pharmaceutical compositions of the disclosure, such as any of the foregoing PDE11A4 inhibitors of the disclosure, for example an isolated fragment of PDE11A4 comprising a GAF-B binding sequence (e.g. SEQ ID NO: 1 or SEQ ID NO: 2), is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of each of the active pharmaceutical ingredients provided in the pharmaceutical compositions of the disclosure, such as any of the PDE11A4 inhibitors of the disclosure, for example an isolated fragment of PDE11A4 comprising a GAF-B binding sequence (e.g. SEQ ID NO: 1 or SEQ ID NO: 2), is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the active pharmaceutical ingredients according to the disclosure is effective over a wide dosage range. For example, in the treatment of adult humans, dosages independently range from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician. The clinically-established dosages of the PDE11A4 inhibitors of the disclosure may also be used if appropriate.

In an embodiment, the molar ratio of two active pharmaceutical ingredients in the pharmaceutical compositions is in the range from 10:1 to 1:10, preferably from 2.5:1 to 1:2.5, and more preferably about 1:1. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20.

In an embodiment, the pharmaceutical compositions described herein, such as any of the PDE11A4 inhibitors of the disclosure, for example an isolated fragment of PDE11A4 comprising a GAF-B binding sequence (e.g. SEQ ID NO: 1 or SEQ ID NO: 2), are for use in the treatment of a disease or disorder associated with cognitive decline. In an embodiment, the pharmaceutical compositions described herein, such as any of the PDE11A4 inhibitors of the disclosure, are for use in the treatment of dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), or cognitive decline associate with spatial memory, other depression-related behaviors, additional anxiety-related behaviors, sensorimotor gating, and social behaviors.

Described below are non-limiting pharmaceutical compositions and methods for preparing the same.

### Pharmaceutical Compositions for Oral Administration

In an embodiment, the disclosure provides a pharmaceutical composition for oral administration containing the active pharmaceutical ingredient or combination of active pharmaceutical ingredients, such as the PDE11A4 inhibitors described herein (e.g. molecules capable of changing the subcellular localization and/or location of PDE11A4, and/or disrupting and/or preventing homodimerization of PDE11A4, and/or degrading PDE11A4), and a pharmaceutical excipient suitable for oral administration.

In some embodiments, the disclosure provides a solid pharmaceutical composition for oral administration containing: (i) an effective amount of an active pharmaceutical ingredient or combination of active pharmaceutical ingredients, and (ii) a pharmaceutical excipient suitable for oral administration. In selected embodiments, the composition further contains (iii) an effective amount of a third active pharmaceutical ingredient, and optionally (iv) an effective amount of a fourth active pharmaceutical ingredient.

In some embodiments, the pharmaceutical composition may be a liquid pharmaceutical composition suitable for oral consumption. Pharmaceutical compositions of the disclosure suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Each of the active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl-lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogs thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In some embodiments, a pharmaceutical composition is provided for injection containing an active pharmaceutical ingredient or combination of active pharmaceutical ingredients, such as a PDE11A4 inhibitors of the disclosure (e.g., molecules capable of changing the subcellular localization and/or location of PDE11A4, and/or disrupting and/or preventing homodimerization of PDE11A4, and/or degrading PDE11A4), for example an isolated fragment of PDE11A4 comprising a GAF-B binding sequence (e.g. SEQ ID NO: 1 or SEQ ID NO: 2), and a pharmaceutical excipient suitable for injection.

The forms in which the compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating an active pharmaceutical ingredient or combination of active pharmaceutical ingredients in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In some embodiments, a pharmaceutical composition is provided for transdermal delivery containing an active pharmaceutical ingredient or combination of active pharmaceutical ingredients, such as PDE11A4 inhibitors of the disclosure (e.g. molecules capable of changing the subcellular localization and/or location of PDE11A4, and/or disrupting and/or preventing homodimerization of PDE11A4, and/or degrading PDE11A4), for example an isolated fragment of PDE11A4 comprising a GAF-B binding sequence (e.g. SEQ ID NO: 1 or SEQ ID NO: 2), and a pharmaceutical excipient suitable for transdermal delivery.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of an active pharmaceutical ingredient or combination of active pharmaceutical ingredients in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445; and 5,001,139, the entirety of which are incorporated herein by reference. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra and PDE11A4 inhibitors of the disclosure (e.g. molecules capable of changing the subcellular localization and/or location of PDE11A4, and/or disrupting and/or preventing homodimerization of PDE11A4, and/or degrading PDE11A4), for example an isolated fragment of PDE11A4 comprising a GAF-B binding sequence (e.g. SEQ ID NO: 1 or SEQ ID NO: 2). Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions of the PDE11A4 inhibitors of the disclosure (e.g. molecules capable of changing the subcellular localization and/or location of PDE11A4, and/or disrupting and/or preventing homodimerization of PDE11A4, and/or degrading PDE11A4), for example an isolated fragment of PDE11A4 comprising a GAF-B binding sequence (e.g. SEQ ID NO: 1 or SEQ ID NO: 2), may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of an active pharmaceutical ingredient or combination of active pharmaceutical ingredients or a pharmaceutical composition thereof can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The active pharmaceutical ingredient or combination of active pharmaceutical ingredients can also be administered intraadiposally or intrathecally.

Exemplary parenteral administration forms include solutions or suspensions of active compound in sterile aqueous solutions, for example, aqueous propylene glycol or dextrose solutions. Such dosage forms can be suitably buffered, if desired.

### Kits

The disclosure also provides kits. The kits include an active pharmaceutical ingredient or combination of active pharmaceutical ingredients, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In selected embodiments, an active pharmaceutical ingredient or combination of active pharmaceutical ingredients are provided as separate compositions in separate containers within the kit. In selected embodiments, an active pharmaceutical ingredient or combination of active pharmaceutical ingredients are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer.

In some embodiments, the disclosure provides a kit comprising a composition comprising a therapeutically effective amount of an active pharmaceutical ingredient (e.g., a PDE11A4 inhibitor of the disclosure, e.g. molecules capable of changing the subcellular localization and/or location of PDE11A4, and/or disrupting and/or preventing homodimerization of PDE11A4, and/or degrading PDE11A4, for example an isolated fragment of PDE11A4 comprising a GAF-B binding sequence, such as SEQ ID NO: 1 or SEQ ID NO: 2), or combination of active pharmaceutical ingredients or a pharmaceutically acceptable salt, solvate, hydrate, cocrystal, or prodrug thereof. These compositions are typically pharmaceutical compositions. The kit is for co-administration of the active pharmaceutical ingredient or combination of active pharmaceutical ingredients, either simultaneously or separately.

In some embodiments, the disclosure provides a kit comprising (1) a composition comprising a therapeutically effective amount of an active pharmaceutical ingredient (e.g., a PDE11A4 inhibitor of the disclosure, for example an isolated fragment of PDE11A4 comprising a GAF-B binding sequence (e.g. SEQ ID NO: 1 or SEQ ID NO: 2), or combination of active pharmaceutical ingredients, and (2) a diagnostic test for determining whether a patient's disease or disorder associated with cognitive decline is a particular subtype of a disease or disorder associated with cognitive decline. Any of the foregoing diagnostic methods may be utilized in the kit.

The kits described above are preferably for use in the treatment of the diseases and conditions described herein. In some embodiments, the kits are for use in the treatment of a disease or disorder associated with cognitive decline. In some embodiments, the kits are for use in the treatment of dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), or cognitive decline associate with spatial memory, other depression-related behaviors, additional anxiety-related behaviors, sensorimotor gating, and social behaviors.

### Dosages and Dosing Regimens

The amounts of the pharmaceutical compositions administered using the methods herein, such as the dosages of PDE11A4 inhibitors of the disclosure (e.g., molecules capable of changing the subcellular localization and/or location of PDE11A4, and/or disrupting and/or preventing homodimerization of PDE11A4, and/or degrading PDE11A4), for example an isolated fragment of PDE11A4 comprising a GAF-B binding sequence (e.g. SEQ ID NO: 1 or SEQ ID NO: 2), will be dependent on the human or mammal being treated, the severity of the disorder or condition, the rate of administration, the disposition of the active pharmaceutical ingredients and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of the pharmaceutical compositions and active pharmaceutical ingredients may be provided in units of mg/kg of body mass or in mg/m² of body surface area.

In some embodiments, the disclosure includes methods of treating a disease or disorder associated with cognitive decline in human subject suffering from the disease or disorder, the method comprising the steps of administering a therapeutically effective dose of an active pharmaceutical ingredient that is a PDE11A4 inhibitor of the disclosure, for example an isolated fragment of PDE11A4 comprising a GAF-B binding sequence (e.g. SEQ ID NO: 1 or SEQ ID NO: 2), to the human subject.

In some embodiments, the disclosure includes methods of treating a disease or disorder associated with cognitive decline in a human subject suffering from the disease or disorder, the method comprising the steps of administering a therapeutically effective dose of an active pharmaceutical ingredient that is a PDE11A4 inhibitor, for example an isolated fragment of PDE11A4 comprising a GAF-B binding sequence (e.g. SEQ ID NO: 1 or SEQ ID NO: 2), to the human subject to inhibit or decrease the activity of PDE11A protein.

In some embodiments, a pharmaceutical composition or active pharmaceutical ingredient is administered in a single dose. Such administration may be by injection, e.g., intravenous injection, in order to introduce the active pharmaceutical ingredient quickly. However, other routes, including the preferred oral route, may be used as appropriate. A single dose of a pharmaceutical composition may also be used for treatment of an acute condition.

In some embodiments, a pharmaceutical composition or active pharmaceutical ingredient is administered in multiple doses. In an embodiment, a pharmaceutical composition is administered in multiple doses. Dosing may be once, twice, three times, four times, five times, six times, or more than six times per day. Dosing may be once a month, once every two weeks, once a week, or once every other day. In other embodiments, a pharmaceutical composition is administered about once per day to about 6 times per day. In some embodiments, a pharmaceutical composition is administered once daily, while in other embodiments, a pharmaceutical composition is administered twice daily, and in other embodiments a pharmaceutical composition is administered three times daily.

Administration of the active pharmaceutical ingredients may continue as long as necessary. In selected embodiments, a pharmaceutical composition is administered for more than 1, 2, 3, 4, 5, 6, 7, 14, or 28 day(s). In some embodiments, a pharmaceutical composition is administered for less than 28, 14, 7, 6, 5, 4, 3, 2, or 1 day(s). In some embodiments, a pharmaceutical composition is administered chronically on an ongoing basis—e.g., for the treatment of chronic effects. In some embodiments, the administration of a pharmaceutical composition continues for less than about 7 days. In yet another embodiment the administration continues for more than about 6, 10, 14, 28 days, two months, six months, or one year. In some cases, continuous dosing is achieved and maintained as long as necessary.

In some embodiments, an effective dosage of an active pharmaceutical ingredient disclosed herein, for example any of the PDE11A4 inhibitors of the disclosure, for example an isolated fragment of PDE11A4 comprising a GAF-B binding sequence (e.g. SEQ ID NO: 1 or SEQ ID NO: 2), is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of an active pharmaceutical ingredient disclosed herein is less than about 25 mg, less than about 50 mg, less than about 75 mg, less than about 100 mg, less than about 125 mg, less than about 150 mg, less than about 175 mg, less than about 200 mg, less than about 225 mg, or less than about 250 mg. In some embodiments, an effective dosage of an active pharmaceutical ingredient disclosed herein is greater than about 25 mg, greater than about 50 mg, greater than about 75 mg, greater than about 100 mg, greater than about 125 mg, greater than about 150 mg, greater than about 175 mg, greater than about 200 mg, greater than about 225 mg, or greater than about 250 mg.

In some embodiments, an effective dosage of an active pharmaceutical ingredient disclosed herein, for example any of the PDE11A4 inhibitors of the disclosure, for example an isolated fragment of PDE11A4 comprising a GAF-B binding sequence (e.g. SEQ ID NO: 1 or SEQ ID NO: 2), is in the range of about 0.01 mg/kg to about 200 mg/kg, or about 0.1 to 100 mg/kg, or about 1 to 50 mg/kg.

In some embodiments, an active pharmaceutical ingredient is administered at a dosage of 10 to 200 mg BID, including 50, 60, 70, 80, 90, 100, 150, or 200 mg BID. In some embodiments, an active pharmaceutical ingredient is administered at a dosage of 10 to 500 mg BID, including 1, 5, 10, 15, 25, 50, 75, 100, 150, 200, 300, 400, or 500 mg BID.

In some instances, dosage levels below the lower limit of the aforesaid ranges may be more than adequate, while in other cases still larger doses may be employed without causing any harmful side effect, e.g., by dividing such larger doses into several small doses for administration throughout the day. Of course, as those skilled in the art will appreciate, the dosage actually administered will depend upon the condition being treated, the age, health and weight of the recipient, the type of concurrent treatment, if any, and the frequency of treatment. Moreover, the effective dosage amount may be determined by one skilled in the art on the basis of routine empirical activity testing to measure the bioactivity of the compound(s) in a bioassay, and thus establish the appropriate dosage to be administered.

An effective amount of the combination of the active pharmaceutical ingredient may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

In some embodiments, the compositions described herein further include controlled-release, sustained release, or extended-release therapeutic dosage forms for administration of the compounds described herein, which involves incorporation of the compounds into a suitable delivery system in the formation of certain compositions. This dosage form controls release of the compound(s) in such a manner that an effective concentration of the compound(s) in the bloodstream may be maintained over an extended period of time, with the concentration in the blood remaining relatively constant, to improve therapeutic results and/or minimize side effects. Additionally, a controlled-release system would provide minimum peak to trough fluctuations in blood plasma levels of the compound.

The disclosure will be further described in the following embodiments, which do not limit the scope of the disclosure described in the claims.

Embodiment 1. An isolated fragment of PDE11A4 comprising a GAF-B binding sequence and comprising or consisting of a polypeptide sequence of SEQ ID NO: 1, or a sequence having at least about 70%, at least about 75%, at least about 80%, at least about 85%, at least about 87%, at least about 89%, at least about 90%, at least about 91%, at least about 92%, at least about 93%, at least about 94%, at least about 95%, at least about 96%, at least about 97%, at least about 98%, or at least about 99% identity thereto.

Embodiment 2. The isolated fragment of Embodiment 1, wherein the isolated fragment comprises and or consists of a polypeptide sequence of SEQ ID NO: 1.

Embodiment 3. An isolated fragment of PDE11A4 comprising a GAF-B binding sequence and comprising or consisting of a polypeptide sequence of SEQ ID NO: 1 or SEQ ID NO: 2, or a sequence having at least about 70%, at least about 75%, at least about 80%, at least about 85%, at least about 87%, at least about 89%, at least about 90%, at least about 91%, at least about 92%, at least about 93%, at least about 94%, at least about 95%, at least about 96%, at least about 97%, at least about 98%, or at least about 99% identity thereto.

Embodiment 4. The isolated fragment of Embodiment 3, wherein the isolated fragment comprises and or consists of a polypeptide sequence of SEQ ID NO: 1 or SEQ ID NO: 2.

Embodiment 5. A polynucleotide encoding an isolated fragment of PDE11A4 comprising a GAF-B binding sequence having an polypeptide sequence of SEQ ID NO: 1, or a sequence having at least about 70%, at least about 75%, at least about 80%, at least about 85%, at least about 87%, at least about 89%, at least about 90%, at least about 91%, at least about 92%, at least about 93%, at least about 94%, at least about 95%, at least about 96%, at least about 97%, at least about 98%, or at least about 99% identity thereto.

Embodiment 6. A polynucleotide encoding an isolated fragment of PDE11A4 comprising a GAF-B binding sequence having an polypeptide sequence of SEQ ID NO: 1 or SEQ ID NO: 2, or a sequence having at least about 70%, at least about 75%, at least about 80%, at least about 85%, at least about 87%, at least about 89%, at least about 90%, at least about 91%, at least about 92%, at least about 93%, at least about 94%, at least about 95%, at least about 96%, at least about 97%, at least about 98%, or at least about 99% identity thereto.

Embodiment 7. A vector comprising the nucleic acid of Embodiment 5 or Embodiment 6.

Embodiment 8. A host cell comprising the polynucleotide of Embodiment 7.

Embodiment 9. The host cell of Embodiment 8, wherein the cell is a mammalian cell.

Embodiment 10. The host cell of Embodiment 9, wherein said host cell is a HT-22 cell, a CHO cell, a HEK-293 cell, or an Sp2.0 cell.

Embodiment 11. A pharmaceutical composition comprising the isolated fragment of any one of Embodiment 1-4, and a physiologically compatible carrier medium.

Embodiment 12. A pharmaceutical composition comprising the isolated fragment of any one of Embodiment 1-4, and a physiologically compatible carrier medium, wherein the amount of the isolated fragment in the composition is a therapeutically effective amount for the treatment or prevention of a disease or disorder alleviated by inhibiting PDE11A4 activity in a patient in need thereof.

Embodiment 13. The pharmaceutical composition of Embodiment 11 or Embodiment 12, wherein the disease or disorder is associated with cognitive decline.

Embodiment 14. The pharmaceutical composition of Embodiment 13, wherein the disease or disorder is selected from dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), and cognitive decline associate with spatial memory, other depression-related behaviors, additional anxiety-related behaviors, sensorimotor gating, and social behaviors.

Embodiment 15. A method of treating or preventing a disease or disorder alleviated by inhibiting PDE11A4 activity in a patient in need thereof, comprising administering to the patient a therapeutically effective amount of the isolated fragment of any one of Embodiment 1-4.

Embodiment 16. A method of treating or preventing a disease or disorder alleviated by inhibiting PDE11A4 activity in a patient in need thereof, comprising administering to the patient a therapeutically effective amount of a pharmaceutical composition of any one of Embodiment 11-14.

Embodiment 17. The method of any one of Embodiment 15 or Embodiment 16, wherein the isolated fragment is administered in a dosage unit form.

Embodiment 18. The method of Embodiment 17, wherein the dosage unit comprises a physiologically compatible carrier medium.

Embodiment 19. The method of any one of Embodiment 15-18, wherein the disease or disorder is associated with cognitive decline.

Embodiment 20. The method of Embodiment 19, wherein the disease or disorder is selected from dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), and cognitive decline associate with spatial memory, other depression-related behaviors, additional anxiety-related behaviors, sensorimotor gating, and social behaviors.

The following examples describe the disclosure in further detail. These examples are provided for illustrative purposes only, and should in no way be considered as limiting the disclosure.

### EXAMPLES

### Example 1: Phosphodiesterase 11A (PDE11A) and its Role in the Neurobiological Substrates of Memory and Social Behaviors

This Example describes data testing the hypothesis that age-related increases in HIPP PDE11A4 occur in a compartmentalized manner and impair social aLTMs. A novel conditional transgenic system that controls the expression of PDE11A4 in a time- and brain region-specific manner by combining overexpressing or knockdown lentiviruses with PDE11A WT and KO mice is used. RNAscope probes are used to delineate nuclear vs. cytosolic localization of PDE11A4 mRNA. In vivo/ex vivo techniques are used to study of PDE compartmentalization- and its functional consequences-rather than the study of artificial FRET-based constructs in in vitro assays.

Given PDE11A4's uniquely restricted expression pattern, the studies not only uncover the neurobiological role of a particular enzyme, they also define the function of an anatomically restricted, molecularly defined-circuit that appears to be uniquely specialized for processing social information. The focus on LTMs for social experiences is relatively unique in the field of learning memory. Most studies tend to utilize contextual/cued fear conditioning, novel object recognition or various spatial learning paradigms (e.g., water maze). Further, what studies have examined the molecular/anatomical mechanisms of social memory have largely focused on short-term memory (STM). The role that age-related increases in PDE11A4 plays in STM, recent LTM (24 hthes after training), and remote LTM (7 days after training) of both aLTMs and rLTMs for social vs. non-social experiences is compared. An Integrative approach includes the use of in vivo, ex vivo, and in vitro approaches, along with viral techniques, permitting assessing translatability of findings across species.

Determining if Preventing or Reversing Age-Related Increases in PDE11A4 can Prevent and/or Rescue Age-Related Impairments in Social aLTM.

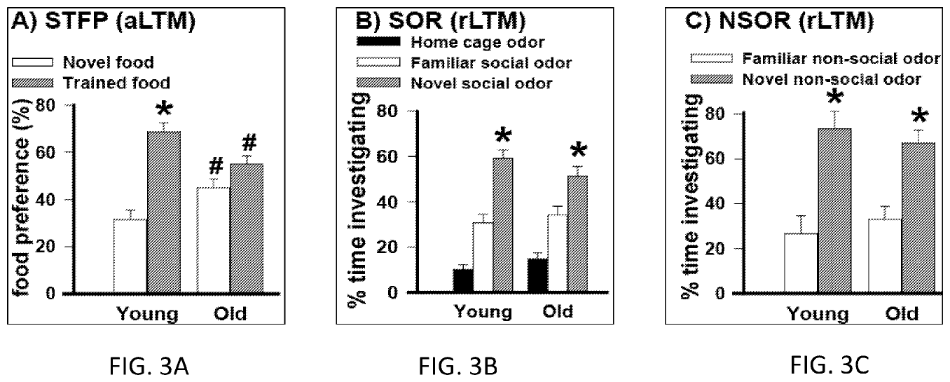

Age-related cognitive decline is not a uniform process, with variability in symptom severity observed across cognitive domains. Human studies have demonstrated that associative long-term memories (aLTMs)—particularly those involving experiences with family and friends—are more susceptible to age-related cognitive decline than are recognition long-term memories (rLTMs). This differential sensitivity of social aLTMs vs rLTMs in mice (FIG. 3) was recapitulated. The findings, in combination with the human studies noted above and a report showing rapid decay of social aLTMs in aged rats, suggests that an enhanced vulnerability of social aLTMs to age-related cognitive decline is conserved across species.

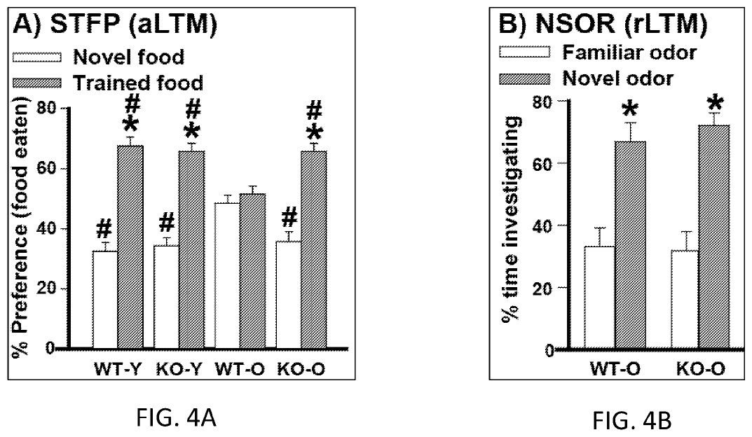

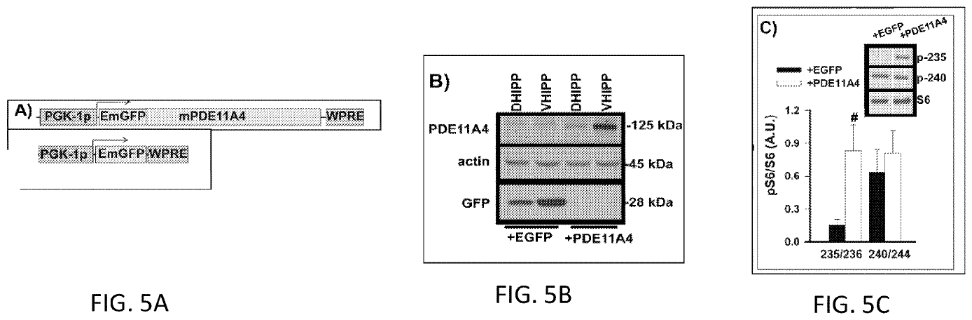

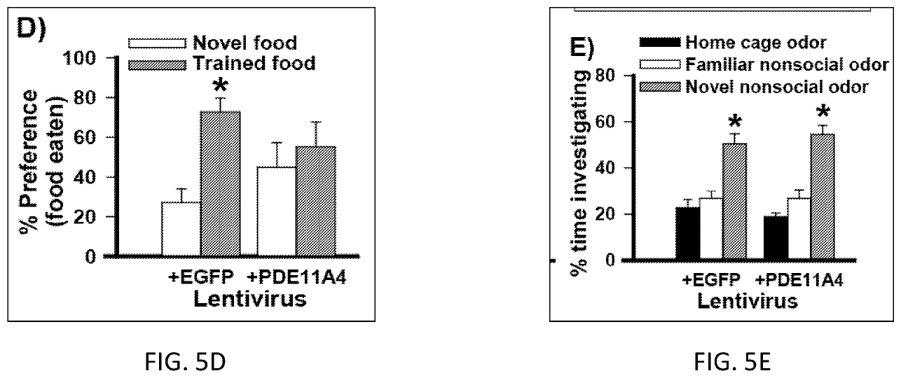

To test the hypothesis that age-related cognitive decline of social aLTMs is driven by the age-related increases in PDE11A4 expression described above (FIGS. 2A-2C), young vs. old PDE11A WT and KO mice in social transmission of food preference (STFP; FIGS. 4A-4B) were compared. While aging severely impairs PDE11A WT mice, old KO mice show robust aLTM for STFP on par with that of young PDE11A WT mice. These findings have been replicated in two large cohorts of male and female mice (no effect of sex; combined data shown in FIGS. 4A-4B), underscoring the reproducibility of the protective effect. Further, in preliminary studies, it has been shown that the protective effect of PDE11A deletion is reversible by acutely overexpressing PDE11A4 in the hippocampus of PDE11A KO mice (i.e., mimicking the state of an old WT; FIGS. 5A-5E). Together, these data show that preventing age-related increases in PDE11A4 is sufficient to prevent age-related cognitive decline of social aLTMs and suggests that more acute manipulations of elevated PDE11A4 expression is meet with equal success.

Based on data disclosed herein, preventing or minimizing age-related increases in PDE11A4 is sufficient to prevent the onset of age-related cognitive decline of social aLTMs (FIGS. 4A-4B). Further, the age-related cognitive decline observed in PDE11A WTs is due to the excessive PDE11A4 that is acutely present in the aged adult hippocampus. Identifying when and where PDE11A4 affects age-related cognitive decline allowed for the discovery of more sophisticated therapeutic approaches to target PDE11A4 for the reversal of aLTM deficits in aged adults or only as a prophylactic agent that prevents the onset of deficits. These results demonstrate that preventing or reversing age-related increases in HIPP PDE11A expression are sufficient to rescue age-related impairments in social aLTM.

### Experimental Approach:

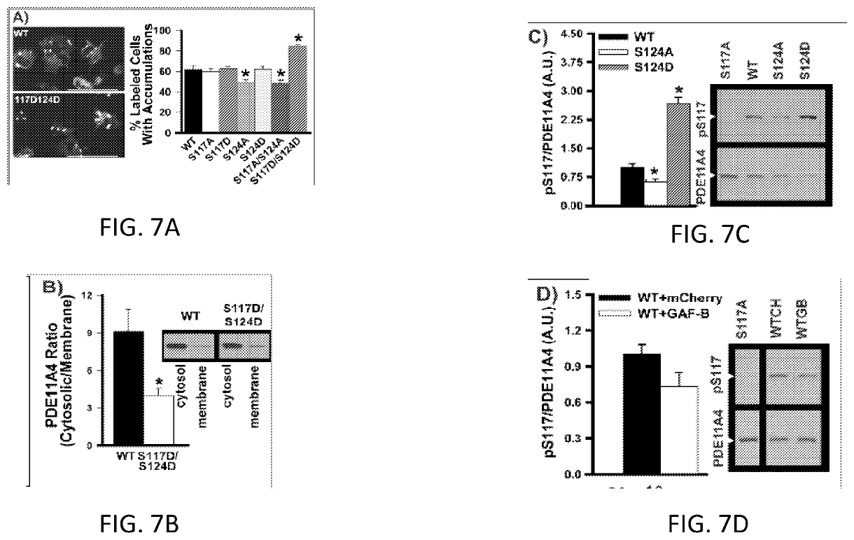

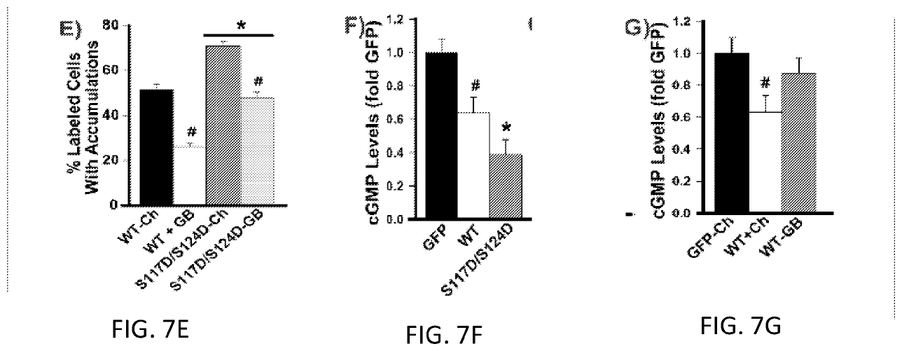

Innovative conditional transgenic system used to prevent, reverse, or mimic age-related increases in HIPP PDE11A4: This system combines PDE11A WT and KO mice with lentivirus constructs that either overexpress (FIGS. 5A-5E). Deletion of PDE11A does not appear to trigger compensatory upregulation of closely related PDEs. In a non-limiting embodiment, integrative studies are conducted with a number of mutant mouse models and lentiviruses. In a non-limiting example, “young” is defined as 2-6 months of age and “Old” is defined as 18-22 months of age since 1) protective effects are observed in PDE11A KO mice as early as 14 months old and 2) it is expected 50% of the colony dies by the age of 24 months (i.e., do not want to introduce a selection bias by preferentially studying older mice with exceptional longevity). In the mimic experiments, PDE11A WT or KO mice are administered a lentivirus containing a control fluorescent protein (EmGFP, as appropriate) or an N-terminal EmGFP-tagged PDE11A4 construct. It was chosen to place the tag at the N-terminus in view of the benign nature of a PDE11A4 N-terminal tag, which have been confirmed (FIGS. 7F-7G). Untreated male and female PDE11A WT, HT and KO mice are used to study the effects of preventing (in the case of the KO) or minimizing (in the case of the HT) age-related increases in PDE11A4 expression (FIGS. 4A-4B).

Viral vectors were delivered using stereotaxic techniques similar to those that have been described previously, except that injections were made directly into the hippocampus. Coordinates were selected to target CA1 and subiculum—the portions of the hippocampus that naturally express PDE11A4 (DHIPP coordinates: AP-1.7 mm, ML+/−1.6 mm, DV-1.4 mm; VHIPP coordinates: AP-3.3 mm, ML+/−3.5 mm, DV-4.4 mm). The ability to virally manipulate PDE11A4 expression has been measured and function in vivo and to measure aLTM and rLTM in virally-treated mice (FIGS. 5A-5E). These data support the hypothesis that reversal/mimicry of age-related increases in PDE11A4 expression in the adult hippocampus is sufficient to rescue/cause deficits in social aLTMs.

Social aLTM using STFP were measured and—to determine the specificity of the effects—1) non-social aLTM using contextual fear conditioning, 2) social rLTM using SOR, and 3) non-social rLTM using NSOR. To date, experiments measuring remote aLTM for STFP (i.e., 7 days post training; FIGS. 4A-4B and 5A-5E) have been prioritized as opposed to short-term memory (STM; 1 hthe post training) or recent long-term memory (24 hours post training) for 2 reasons. First, a study in rats suggested that aging impairs aLTM for STFP at more remote time points but not immediately after training. Second, adolescent and young adult PDE11A KO mice show intact STM and remote LTM (FIGS. 4A-4B and 5A-5E), but impaired recent LTM for social experiences. The studies to date indicate that PDE11A KO mice exhibit transient amnesia by virtue of expediting systems consolidation, which temporarily “misplaces” the memory but ultimately results in a strengthened memory trace in the cortex. While it remains to be determined if old PDE11A KO mice similarly show a form of transient amnesia (intact STM, impaired recent LTM, improved remote LTM), such an effect on systems consolidation could explain why old KOs are protected from age-related cognitive decline (see below for Arc mapping study that tests for this possibility). That said, Young PDE11A heterozygous (HT) mice do show recent LTMs for social experiences, and yet old PDE11A HTs are still protected against age-related cognitive decline of aLTMs for STFP (HT-O: novel food, 35.9±3.9%; trained food 64.1±3.9%; P<0.001). Thus, transient amnesia is not required to see the protective effects of PDE11A deletion on remote aLTMs in aged mice.

Moving forward, young vs. old WT, HT, and KO PDE11A mice are compared at all 3 time points (STM, recent LTM, and remote LTM) to determine the effects of age-related increases in PDE11A4 at each memory phase. In addition to behavior, ex vivo studies are conducted. Functional activation of neural circuits was mapped using in situ hybridization for the activity-regulated gene Arc, as has been previously disclosed. In so doing, the circuit was identified (including nodes of activity and functional connectivity amongst nodes) that is engaged by aged PDE11A KO mice during the successful retrieval of a social aLTM. It is then determined if the circuit engaged by an aged PDE11A KO shares similarities with or diverges from that of young PDE11A WT mice. This approach shows whether preventing age-related increases in PDE11A prevents/delays the onset of aging pathophysiology or if it simply enables the brain to adopt a compensatory strategy for achieving equivalent behavioral performance (i.e., engages cognitive reserve). It is also determined if preventing/reversing age-related increases in PDE11A4 is sufficient to prevent/reverse age-related changes in phosphorylation of CREB, GluR1, and CaMKII that it has been reported to occur in the hippocampus. Cause-and-effect studies determine which of these downstream events may be sufficient to drive age-related cognitive decline of social aLTMs.

In conducting these ex vivo studies, cannula tracks and PDE11A4 expression levels are verified in each virally-treated subject. If a viral delivery is determined to have failed (e.g., cannula tracks miss the hippocampus, PDE11A4 expression not changed) data from that subject are dropped. Approximately equal numbers of male and female offspring are used in all studies (see infra for specific n's). All experiments are counterbalanced for sex and genotype, but data are collected by an experimenter blind to genotype.

### Results

Based on the fact that PDE11A HT and KO mice are protected against age-related decline of aLTMs (FIGS. 4A-4B), it was determined that age-related increases in PDE11A4 expression are deleterious. Although not wishing to be bound by any particular theory, the fact that the protective effect of the KO was acutely reversed suggests that the deleterious effects of PDE11A4 were due to the acute presence of elevated PDE11A4 in the aged brain (as opposed to a cumulative effect of chronic overexpression). Knocking down elevated PDE11A4 expression in the aged hippocampus was as effective as preventing age-related increases in PDE11A4 expression. This is based on two primary results: 1) virally overexpressing PDE11A4 expression in old PDE11A KO or young WT mice (i.e., mimicking the state of an old WT) was sufficient to cause deficits in social aLTMs, and 2) virally knocking down PDE11A4 expression in aged WTs was sufficient to rescue social aLTM deficits. It was also determined that aged PDE11A KO mice exhibit transient amnesia for social memories as it has been observed in young KOs. Old PDE11A KO mice showed intact or improved recent LTM, suggesting that the function of PDE11A4 evolves across the lifespan.

Identify the Circuit, Cell-Type and Subcellular Domain where PDE11A4 is Upregulated with Age and the Signal Driving this Compartmentalized Effect.

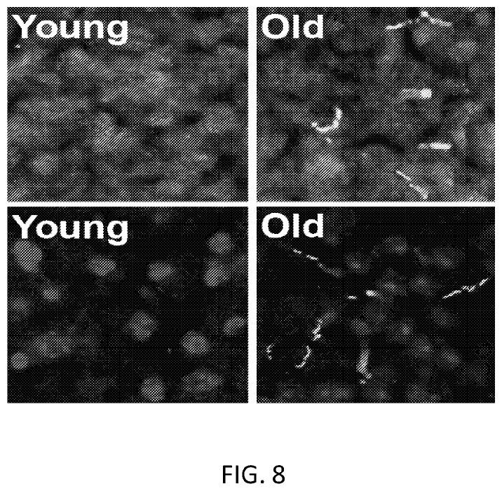

Intramolecular signals have been identified that alter the trafficking of PDE11A4, including homodimerization and N-terminal phosphorylation (FIGS. 7A-7G). As such, it is important to determine 1) if the rate/nature of PDE11A4 post-translational modifications change with age and, as a result, 2) if age-related increases in PDE11A4 expression occur in a compartment-specific manner (i.e., if excessive PDE11A4 is aberrantly trafficked). In a preliminary study, PDE11A4 were mapped expression in young vs old C57BL/6J and BALB/cJ mice by immunofluorescence and found in both strains and sexes that the excessive expression of PDE11A4 protein that occurs with age appears to accumulate in short “filamentous structures” (FIG. 8). These PDE11A4-filled structures are rarely seen in young mice but are abundant in CA1, AHi, and especially the subiculum of aged mice. The preliminary study also shows that aging dramatically increases phosphorylation of PDE11A4 at serines 117 and 124 (pS117/pS124) and that pS117/pS124 is found almost exclusively in the pools of PDE11A4 that accumulate in these filamentous structures (FIG. 8). This in vivo finding is consistent with the in vitro studies showing that the phosphomimic mutation S117D/S124D increases accumulation of PDE11A4 while the phosphoresistant mutation S117A/S124A has the opposite effect (FIG. 7A).

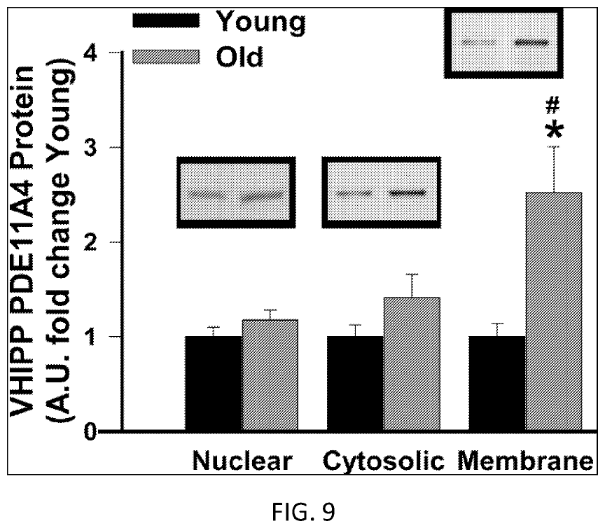

A preliminary biochemical fractionation experiment also points to compartment-specific effects. Age-related increases in VHIPP PDE11A4 occur primarily in the membrane fraction as opposed to the cytosol or nucleus, suggesting a mislocalization of the overexpressed PDE11A4 (FIG. 9). Although not wishing to be bound by any particular theory, this mislocalization is likely driven by the increase in PDE11A4-pS117/pS124 because the in vitro studies show that S117D/S124D shifts PDE11A4 from the cytosol to the membrane (FIG. 7B). Not only does S117D/S124D change the localization of PDE11A4, it also selectively increases PDE11A4 cGMP hydrolytic activity, thus causing deficits in cGMP that mimic aging (FIG. 7F). Together, these data suggest that age-related increases in PDE11A4 expression are compounded by increased cGMP hydrolytic activity and a mislocalization of that aberrant activity that is driven by phosphorylation at S117/S124.

Unlike S117D/S12D, disrupting PDE11A4 homodimerization reduces PDE11A4 accumulation and shifts PDE11A4 from the membrane back to the cytosol. Importantly, disrupting PDE11A4 homodimerization also reduces PDE11A4 cGMP hydrolytic (FIG. 7G), consistent with the fact that it reduces pS117/pS124-PDE11A4 (FIG. 7D). As such, studies here determines if preventing phosphorylation at S117/S124 or disrupting homodimerization are sufficient to prevent/reverse 1) age-related accumulation of PDE11A4 in filamentous structures and/or the membrane and 2) age-related deficits in social aLTMs.

Previously it was shown that PDE11A4 protein expression increases in hippocampal lysates, but the specific compartment within which this increase occurs has yet to be identified. It is possible that this excess PDE11A4 may not be globally distributed, but rather discretely localized or even ectopically expressed in cell types or subcellular domains that are normally void of PDE11A4. Defining the signals that control age-related changes in PDE11A4 trafficking enables a more sophisticated targeting of compartment-specific localizations of the enzyme as opposed to overall catalytic activity, which is preferred since eliminating all PDE11A catalytic activity influences social preferences and impairs recent LTMs for social experiences.

These results demonstrated that age-related increases in pS117/pS124 cause PDE11A4 to accumulate ectopically, contributing to age-related decline of social aLTMs.

### Experimental Approach

Series 1: Identifying the location of age-related increases in PDE11A4 at the level of circuit, cell type, and subcellular domain and the intramolecular signals driving those discrete changes. To establish the conservation of effects across species, these experiments utilize brains from young vs old 1) C57BL/6 and BALB/cBy mice, 2) Fischer 344 and Brown Norway rats, and 3) rhesus monkeys (all obtained from the NIA Rodent Colonies or Primate Tissue Bank). Mice and rats are available as live animals and, thus, are tested for remote LTM of STFP, SOR, and NSOR to confirm a selective age-related cognitive decline of social aLTMs. Monkey tissue is available only from post mortem stock and, thus, subjects are not cognitively phenotyped. As noted above, age-related expression changes in PDE11A4 do appear to be conserved between rodents and primates at the level of total HIPP mRNA levels (rat: ; human: FIG. 2B); this is verified at the level of PDE11A4 protein expression and compartmentalization. Tissue from PDE11A KO mice are processed in parallel as a negative control.

Defining changes at the level of circuit and cell type. As per the published techniques, the first hemisphere (rodents) or block of tissue (primate) are kept intact for processing by in situ hybridization and IF in order to determine in which hippocampal subfields age-related increases in PDE11A4 mRNA, protein expression, and phosphorylation occur (e.g., CA1 vs. subiculum or stratum radiatum vs. stratum pyramidale). As noted above, changes in the subcellular location of a PDE would impact function—but so would changes at the level of circuit or cell type. For example, changes in subiculum would indicate an effect on retrieval mechanisms; whereas, changes in CA1 would indicate an effect on input integration. More specifically, changes in CA1 dendrites of stratum radiatum proximal to the cell body indicates modulation of CA3 input signals, while changes in distal CA1 dendrites indicates modulation of entorhinal cortex input signals. As described above, the age-related increases in PDE11A4 expression most strikingly occur in the filamentous structures; however, the preliminary study also showed that a subset of sporadically distributed neurons in the VHIPP stratum pyramidale exhibit increased PDE11A4 expression around the cell body. The sporadic nature of these cell bodies raises the possibility that these neurons reflect either a specific subtype of inhibitory interneuron or neurons that send projections to a discrete brain region. In young mice, it was found only PDE11A4 expressed in excitatory neurons of CA1, subiculum, and the AHi. The preliminary IF study described above verifies that PDE11A4 expression continues to be restricted to these subfields in old mice but it has not been verified that PDE11A4 is only expressed in excitatory neurons in old mice. If PDE11A4 were to become ectopically expressed in inhibitory interneurons with age, this has significant functional consequences for the excitatory-inhibitory balance of the CA1 circuit

As such, co-labeling studies here determine in which cell types PDE11A4 is expressed in the aging brain and if a specific subtype segregates with cells showing increased expression of PDE11A4 around the cell body. If PDE11A4 continues to only be expressed in excitatory neurons during aging, then retrograde tracer studies using stereotaxically-delivered fluorogold are conducted to determine if this neuronal subpopulation segregates based on their projections. Of particular interest are anterior cingulate cortex, entorhinal cortex, and retrosplenial cortex as these brain regions show heightened activation in the PDE11A KO during retrieval of enhanced remote social aLTM. Nucleus accumbens (NAcc) are also of interest given that a subset of PDE11A4-expressing neurons project to NAcc and ventral CA1-NAcc projections are required for social aLTM.

Defining changes at the level of subcellular domain. To determine which subcellular domain constitutes the “filamentous structures” in which PDE11A4 accumulates with age, the slides collected above along are used with the previously published techniques to co-label for PDE11A4 and various markers, including those for axons, dendrites, glia, perineuronal nets, collagen, etc. Biochemical fractionation is conducted using DHIPP and VHIPP dissected from the second hemisphere of the rodents as well as anterior and posterior HIPP tissue samples from primates, with resulting fractions run on denaturing or native Western blots, all as per the published techniques. Fractionation experiments determine 1) if age-related increases in PDE11A4 protein expression occur preferentially in membrane vs. cytosolic vs nuclear fractions (denaturing blots; FIG. 9) and 2) if age-related increases in PDE11A4 protein expression are accompanied by increased PDE11A4 homodimerization (native blots). Increased PDE11A4 homodimerization, along with pS117/pS124, is expected to preferentially upregulate PDE11A4 expression in the VHIPP membrane as was observed in the preliminary study (FIG. 9). Together, experiments identifying where age-related increases in PDE11A4 expression occur at the level of subcellular compartments, cell types, and circuits and defines the potential intramolecular signals driving those compartment-specific effects.

Identifying mechanisms by which PDE11A4 expression patterns in old mice is restored to those of young mice. Previously, it was shown that expression of an isolated GAF-B domain (the domain required for PDE11A4 homodimerization) was sufficient to disrupt PDE11A4 homodimerization by acting as a negative sink. Further, it was shown that reduced levels of PDE11A4 homodimerization are associated with 1) reduced accumulation of PDE11A4, 2) a shifting of PDE11A4 from the membrane to the cytosol, and 3) reduced expression of PDE11A4 due to increased proteolysis. While not wishing to be bound by any particular theory, these studies suggest that age-related changes in PDE11A4 expression/compartmentalization may be prevented/reversed by reducing levels of PDE11A4 homodimerization.

In vitro and in vivo experiments are conducted to determine if the isolated GAF-B domain can 1) reduce phosphorylation of PDE11A4 at S117/S124, 2) prevent/reverse the enhanced accumulation that is seen with the S117D/S124D mutations, and 3) prevent/reverse the increased membrane expression that is seen with S117D/S124D. In preliminary studies, it was shown that disrupting homodimerization in vitro reduces pS117/pS124 of PDE11A4WT and restores trafficking patterns of S117D/S124D to that of PDE11A4WT (FIGS. 7D-7E). In vivo studies utilizing lentiviruses is conducted following the general approach described above. The isolated GAF-B domain (or control) are chronically expressed via lentiviral injection to the HIPP of old PDE11A WT mice to determine if disrupting PDE11A4 homodimerization is sufficient to restore aged PDE11A4 phosphorylation, expression, and trafficking patterns to those observed in young mice and, in so doing, prevent age-related decline of social aLTMs. Importantly, the lentiviruses express for at least 3 months, allowing for chronic manipulations. The GAB-B lentivirus have been obtained and it has been confirmed that it expresses in vivo. The effects of virally overexpressing S117A/S124A vs PDE11A4WT (vs. a control lentivirus) are compared in HIPP of old PDE11A KO mice. In so doing, it is determined if preventing phosphorylation of S117/S124 is sufficient to block the accumulation of PDE11A4 that is seen with high levels of endogenous PDE11A4 expression (FIG. 8) or viral overexpression of PDE11A4WT. Social aLTM using STFP is also assessed to determine if preventing phosphorylation of S117/S124 blocks the ability of PDE11A4 overexpression to impair social aLTM (FIGS. 5A-5E). Together, these studies help understand how/why age-related increases in PDE11A4 lead to age-related decline of social aLTM and, in so doing, identify novel therapeutic mechanisms by which the ectopic localization of PDE11A4 that occurs with age is addressed.

### Results

Based on the consistencies of the findings in mouse, rat, and human tissue to date (FIG. 2B), it is believed age-related changes in PDE11A4 protein expression and compartmentalization are highly conserved across species. Based on the preliminary studies, it is anticipated that aging are associated with increased PDE11A4 expression and phosphorylation at S117/S124 in select compartments, notably the membrane fraction of VHIPP, select cell bodies in the superficial layer of CA1, and the filamentous structures that are most enriched in the subiculum. Although aging and S117D/S124D appear to regulate PDE11A4 in the opposite direction of disrupted homodimerization, it is do not believed aging are associated with increased homodimerization because in vitro studies show that S117D/S124D does not promote homodimerization. Nonetheless, it is anticipated that disrupting homodimerization remedies age-related deficits in PDE11A4 expression and compartmentalization by reducing phosphorylation of pS117/pS124 (FIG. 7D) and promoting proteolysis. Thus, it is expected that expression of the isolated GAF-B domain or S117A/S124A are sufficient to prevent age-related cognitive decline of social aLTMs.

It is not anticipated significant technical hurdles that prevents completion of the proposed experiments. In a non-limiting example, co-labeling studies are insufficient to identify the nature of the “filamentous structures,” despite the fact that there are a large number of validated antibodies that label axons, dendrites, etc.; and electron microscopy (EM) is used for study of ultrastructure. It has been determined the custom PDE11A4 antibody works with the EM fixative acrolein in immunohistochemistry, which is a strong predictor of an ability to work in a full EM protocol. In a non-limiting embodiment, tissue is labeled from EmGFP-PDE11A4 infected PDE11A KOs since overexpressed EmGFP-PDE11A4 also accumulates in filamentous structures in KOs and has been previously validated a GFP antibody in EM using EmGFP-PDE11A4 transfected COS-1 cells.

Westerns blots have been conducted and in situ hybridization in human tissue, so it is not anticipated unsurmountable difficulties in adapting the techniques for monkey tissue. In a non-limiting example, the NIA Primate Tissue bank tracks only age and general health of the monkeys, not their cognitive abilities. It was possible to detect age-related increases in PDE11A4 mRNA in human hippocampus without information regarding cognitive abilities (FIG. 2B), so it is believed the same are possible with monkey tissue. Thus, it is believed the proposed examination of this readily available monkey tissue is a worthwhile first step towards establishing the translatability of the findings, which is of particular concern when measuring age-related changes in the brain. Primates may show PDE11A4 expression in additional/alternative hippocampal subfields (e.g., DG) or cell types (e.g., inhibitory interneurons) than mice and rats, which is important given that location infers function. In this case, the functional consequences of virally expressing PDE11AWT vs. a control virus in those additional subfields or specific cell types (i.e., by using a cell type-specific promoter) using WT mice are interrogated.

### Identify Molecular Mechanisms Driving Age-Related Increases in PDE11A4 Expression

As described above, the spatially restricted nature of PDE11A4 expression is maintained across the lifespan; however, steady-state levels of PDE11A4 protein expression within the HIPP steadily increase. Increases in steady state levels of PDE11A4 protein could result from a number of mechanisms including increased rates of transcription and/or translation or increased stability of the transcript and/or protein. It has been shown previously that PDE11A mRNA expression is also increased in the aged rat brain and more recently discovered that PDE11A mRNA increases with age in the human hippocampus (FIG. 2B). Thus, age-related increases in PDE11A4 protein expression appear to be driven, at least in part, by increases in PDE11A4 transcription and/or transcript stability.

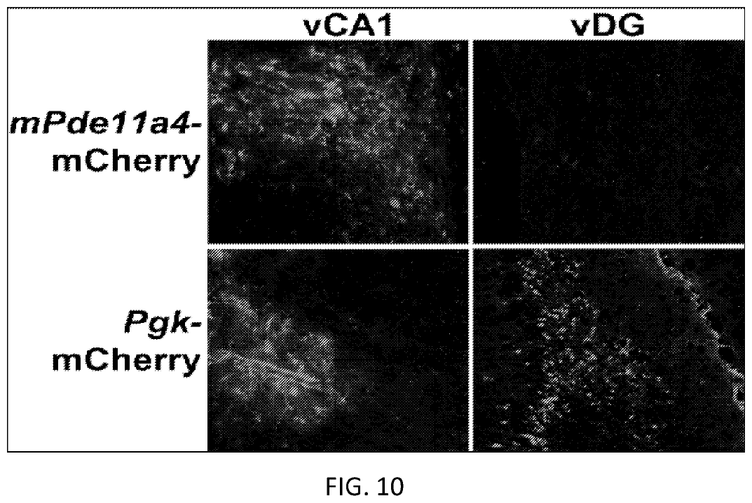

In general, transcription of a given gene is controlled by a core promoter, promoter-proximal elements, as well as enhancers or silencers. Although the core promoter falls within 30 base pairs (bp) of the transcription initiation site (TIS) and the promoter-proximal elements fall within 200 bp of the TIS, enhancers and silencers can fall anywhere within 50 kB of the TIS. A number of studies have shown that a gene promoter is sufficient to drive transcription of a gene in a tissue-specific manner. Indeed transgenic technology makes great use of this fact by ligating the promoter of a tissue-specific gene to the coding sequence of a transgene in order to spatially restrict expression of that transgene to a desired tissue. Putative promoters have been described for hPDE11A4, hPDE11A3, and hPDE11A1 in the 1200 bps upstream of their respective TISs. Thus, to measure transcriptional activity of the mPde11a4 promoter, a lentivirus construct that uses the 1200 bps upstream of the mPde11a4 TIS to drive expression of the mCherry reporter (mPde11a4-mCherry) was developed. The decision to take a lentiviral approach is based on a prototype study by Chhatwal and colleagues. In a recent pilot study, infusion of a control Pgk-mCherry reporter resulted in mCherry expression in both CA1 and DG; however, infusion of the mPde11a4-mCherry reporter resulted in mCherry expression only in CA1 (FIG. 10). Although not wishing to be bound by any particular theory, this result suggests the tool faithfully tracks transcriptional activity of the mPde11a4 promoter, and so it is used it to compare PDE11A4 transcriptional activity in young vs old rodents.

### Experimental Approach

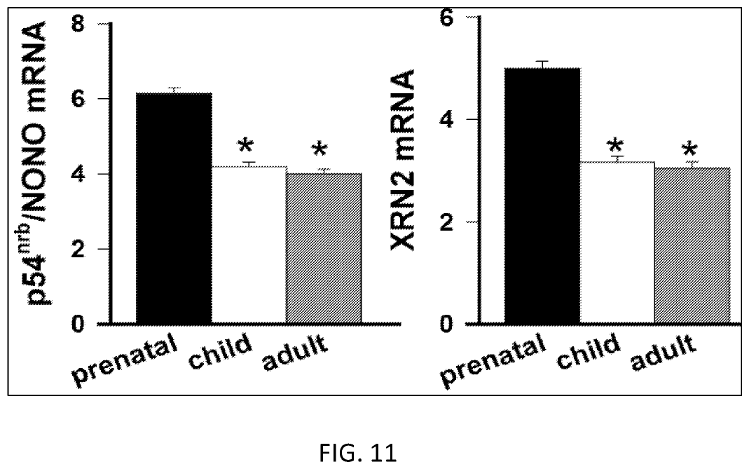

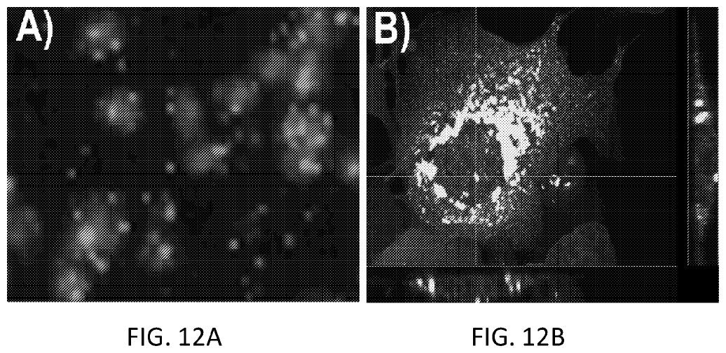

Assessing rates of PDE11A4 mRNA degradation by its known exoribonuclease (XRN2). First, in situ hybridization and Western blots are conducted on young vs. old HIPP tissue collected as described above to determine if age-related decreases in p54^{mb}/NONO and XRN2 are observed in rodents and rhesus monkeys as they are in humans (FIG. 11). Immunoprecipitation are also conducted using total homogenates using methods described above. Antibodies are used against p54^{mb}/NONO and XRN2 to perform pull downs and then RT-PCR is conducted for PDE11A4 mRNA. It is determined if old animals show less binding of p54^{mb}/NONO and XRN2 to the PDE11A4 transcript than do young animals. Finally, nuclear and cytoplasmic RNAs are isolated from young and aged rodents using the Ambion Paris system according to manufacturer's instructions (Life Technologies) and, again, RT-PCR for PDE11A4 mRNA are conducted. This allows for determination if aging is associated with heightened accumulation of PDE11A4 mRNA in the nucleus vs. the cytosol, which indicates impaired degradation by the p54^{mb}/NONO-XRN2 complex. In the event of finding a relative increase in nuclear PDE11A4 mRNA expression, brain sections collected as described above are used to conduct confirmatory in situ hybridization experiments using RNAscope probes (ACD). RNAscope probes enable single molecule-level resolution (see FIG. 12A) and when combined with confocal imaging (FIG. 12B), enable the qualitative assessment of PDE11A mRNA expression in the nucleus vs. the cytosol.

### Results

It is expected to find increased mCherry expression in old vs young rodents only when they are injected with the mPde11a4-mCherry into hippocampal subfields that show increased PDE11A4 mRNA expression. In a non-limiting example, if the constitutive Pgk-mCherry reporter also shows increased expression in the aged hippocampus, then this result suggests a more global upregulation of transcriptional activity, or a potential indirect effect of the mCherry itself, as opposed to a selective upregulation of PDE11A4 transcriptional activity. In a non-limiting example, the finding that aging is associated with increased PDE11A4 transcription or transcript stability does not rule out the possibility that aging is also associated with increased rates of translation or protein stability, and the possibility of age-related reductions in sumoylation or ubiquitination of PDE11A4 is further examined.

The pilot data suggests the 1200 bps upstream of the PDE11A4 TIS is sufficient to control the transcription of mPde11a4 in terms of its spatial distribution, but it is possible that aging also/alternatively could influence rates of transcription by differentially engaging enhancer or silencing motifs within 50 kB of the TIS. In a non-limiting example, to identify cis-acting elements beyond the promoter region that might control PDE11A4 transcription, a BAC transgenic approach is adopted as previously described by Koppel and colleagues. BAC transgenic mice are generated that express a fluorescent protein under the control of a minimal promoter coupled with various combinations of PDE11A4 exons, introns, 5′ upstream sequences, and 3′ downstream sequences.

Further, exoribonucleases are not the only mechanism regulating transcript stability; microRNAs, along with other noncoding RNAs, play an important role as well. In another non-limiting example, the focus is to identify non-coding RNAs that regulate PDE11A4 expression. Of primary interest are miR-375. This is one of only 4 microRNAs that is predicted by TargetScan to target the PDE11A4 transcript. Interestingly, miR-375 expression decreases with age in the mouse brain, which is consistent with the observed age-related increases in PDE11A4.

Statistical Analyses: Power analyses are conducted post hoc to confirm that n=10/sex/group provides sufficient power for in vivo studies and n=4 biological replicates/group (×3 experiments) provides sufficient power for cell culture experiments (as they have in the past). In Western blot experiments, samples generally must span multiple blots (particularly in fractionation studies). Therefore, to account for non-specific technical differences across blots (e.g., transfer or antibody binding efficiency, film exposures etc.), all biochemical data are normalized as a fold change of the control group on a given blot, as have been previously described. All datasets meeting normality and equal variance assumptions are tested by parametric statistics. Those datasets not meeting these assumptions are tested by nonparametric statistics. In general, data are analyzed by multifactorial ANOVAs or by repeated measure ANOVAs where appropriate to account for multiple comparisons. For example, in vivo data are analyzed for effects of sex, genotype, lentiviral treatment (in addition to assay-specific factors, such as food type). Statistical outliers (>2 standard deviations from the mean) are dropped from analyses, as previously described. Significant ANOVAs are followed by Student Newman-Keuls post hoc tests, with significance determined as P<0.05. Data in figures are plotted as means±SEMs.

### Data Rigor

The rationale is based on both mouse and human data with follow-up with studies in mice, rats and monkeys, which strengthens the rigor of this proposal. Further, the primary findings have been replicated across multiple cohorts of mice. Achieving robust and unbiased results: Genetically modified mice are genotyped a priori to enable proper counterbalancing of experimental run lists; however, experimenters are blind to genotype at the time of data collection. Genotypes are then reconfirmed post death by Western blot or in situ hybridization. Physical parameters are counterbalanced across subjects (e.g., which is the “trained” spice and which is “novel”). Biological Variables: both males and females were tested for effect of sex.

Since cohorts of mice are aged up, these experiments largely conducted in parallel across years 1-5. One exception is that some experiments slightly lag behind as species selection depends on the outcome of in situ hybridization studies described herein.

These innovative studies provides much needed insight into the fundamental mechanisms of age-related cognitive decline as well as the function and regulation of PDE11A4, including establishing whether PDE11A represents a therapeutic target not only for preventing age-related decline of social aLTMs but also for reversing deficits. In a non-limiting example, the effects of excessive PDE11A4 on acquisition vs. consolidation. vs. retrieval of remote social aLTMs are studied as well as those aimed at understanding the system-level mechanism by which altered cyclic nucleotide signaling in the VHIPP can impair social aLTMs (e.g., by compromising the integrity of neuronal ensembles encoding the memory engram in the hippocampus vs. cortex). Studies also determine if excessive PDE11A4 is problematic simply by virtue of increased steady state levels that are ubiquitously distributed or by virtue of a discretely localized upregulation or even an ectopic expression of PDE11A4. In a non-limiting example, the effect of modulating PDE11A4 function within specific cell-types (e.g., excitatory vs. inhibitory) or sub-region (e.g., subiculum vs. CA1) is examined. In addition, the upstream signaling events are delineated, including the specific kinases that lead to age-related increases in the phosphorylation of PDE11A4. The fact that aging changes the compartmentalization of PDE11A4 provides a biologically-relevant framework for exploring the functional impact of PDE11A post-translational modifications and protein-protein binding events. Defining the signals that control age-related changes in PDE11A4 trafficking enables a more sophisticated targeting of compartment-specific localizations of the enzyme as opposed to overall catalytic activity, which is preferred since eliminating all PDE11A catalytic activity influences social preferences and impairs recent social LTMs. Studies also identify the mechanism(s) by which PDE11A4 expression is upregulated with age. In a non-limiting example, if age-related increases in PDE11A4 expression are driven by increased transcription, additional studies can be performed to seek to identify the transcription factors responsible. Identifying the mechanism driving age-related increases in PDE11A4 open up new avenues for therapeutically restoring normal levels of expression. Overall, the results provide proof of principle for pursuing PDE11A4 as a novel therapeutic target. PDE11A is a highly druggable enzyme and it is positioned to selectively control cyclic nucleotide signaling in a molecularly-defined circuit that specifically regulates social LTMs, without affecting signaling elsewhere. This may relieve age-related impairments in aLTM without causing unwanted side effects. Importantly, PDE11A exhibits all properties the pharmaceutical industry believes an ‘ideal’ drug target should have. The fact that PDE11A is a realistic candidate for drug development increases the value of understanding its biological function.

### Example 2: Role of Cyclic Nucleotide Signaling in Age-Related Decline of Social Memories

This Example describes studies where it is hypothesized that age-related increases in HIPP PDE11A4 occur in a compartmentalized manner and impair social aLTM.

Determine if blocking age-related increases in PDE11A4 can prevent and/or rescue age-related impairments in social aLTM. It is not known if age-related increases in hippocampal PDE11A4 expression reflect a physiological breakdown or an attempt at protective compensation. In studies, PDE11A WT mice show age-related impairments in social transmission of food preference (STFP) aLTM but PDE11A KO mice do not. Further, mimicking the condition of an old WT by overexpressing PDE11A4 in the hippocampus of PDE11A KO mice impairs STFP aLTM. It is hypothesized that preventing/reversing age-related increases in PDE11A expression are sufficient to prevent/reverse age-related impairments in social aLTM. An innovative conditional transgenic system is used to prevent, reverse, or mimic age-related increases in HIPP PDE11A4 and determine the effect on social aLTM vs non-social aLTM and social/nonsocial rLTM, neural circuit activation, as well as biochemical endpoints widely reported to change with age (e.g., CREB). These studies determine if PDE11A represents a therapeutic target not only for preventing age-related decline of social aLTMs but also for reversing deficits.

Identify the circuit, cell-type and subcellular domain where PDE11A4 is upregulated with age and the signal driving this compartmentalized effect. Previously it was shown that PDE11A4 protein expression increases with age in HIPP lysates. It is possible that this excess PDE11A4 may not be globally distributed, but rather discretely localized or even ectopically expressed in cell types or subcellular domains that are normally void of PDE11A4. In studies, biochemical fractionation shows PDE11A4 increases with age preferentially in membrane vs cytosol and nucleus. Immunofluorescence shows PDE11A4 ectopically accumulates in filamentous structures in the aged HIPP, which are rarely seen in young HIPP. Further, this pool of accumulated PDE11A4 is uniquely phosphorylated at serines 117 and 124. In vitro, phosphomimic mutation of S117 and S124 drives accumulation of PDE11A4, while phosphoresistant mutations or blocking homodimerization reduces this accumulation.

In vivo studies are conducted to 1) quantify circuit, cell-type, and subcellular domain-specific age-related increases in PDE11A4 expression/phosphorylation in rodents and primates (for translatability) and 2) determine if blocking phosphorylation of S117/S124 or disrupting PDE11A4 homodimerization in mice can prevent/reverse ectopic trafficking of PDE11A4 and social aLTM deficits. It is hypothesized that age-related increases in pS117/pS124 drive ectopic localization of PDE11A4, contributing to age-related decline. Outcome: Defining the signals that control age-related changes in PDE11A4 trafficking enables a more sophisticated targeting of compartment-specific localizations of the enzyme as opposed to its total catalytic activity.

Identify molecular mechanisms driving age-related increases in PDE11A4 expression. Age-related increases in steady state levels of PDE11A4 protein could be driven by an increase in transcript/protein generation and/or transcript/protein stability. In studies, it was found that PDE11A4 mRNA is increased in aged vs. young rat HIPP and demented vs. nondemented human HIPP. It is hypothesized that age-related increases in PDE11A4 protein expression are driven by increased rates of transcription and/or transcript stability. The novel PDE11A4-promoter reporter is used to measure transcription in HIPP of aged vs. young mice. In addition, rates of PDE11A4 mRNA degradation are assessed by its known exoribonuclease (XRN2). By identifying the mechanism driving age-related increases in PDE11A4 new avenues are open for therapeutically restoring normal levels of expression.

These innovative studies provides much needed insight into PDE11A function/regulation and broaden the understanding of age-related cognitive decline via the interrogation of a very specific molecularly-defined circuit that appears to selectively regulate social memories and their decline.

### Example 3: Biologic that Disrupts PDE11A4 Homodimerization in Hippocampus CA1 Reverses Age-Related Proteinopathies in PDE11A4 and Age-Related Decline of Social Memories

This Example describes studies demonstrating that age-related increases in phosphodiesterase 11A (PDE11A), an enzyme that degrades 3′,5′-cAMP/cGMP and is enriched in the ventral hippocampal formation (VHIPP), drive age-related cognitive decline (ARCD) of social memories. In the VHIPP, age-related increases in PDE11A4 occur specifically within the membrane compartment and ectopically accumulate in filamentous structures termed ghost axons. Previous in vitro studies show that disrupting PDE11 homodimerization by expressing an isolated PDE11A-GAFB domain that acts as a “negative sink” for monomers selectively degrades membrane-associated PDE11A4 and prevents the punctate accumulation of PDE11A4. Therefore, it was determined if disrupting PDE11A4 homodimerization in vivo via the expression of an isolated PDE11A4-GAFB domain would be sufficient to reverse 1) age-related accumulations of PDE11A4 in VHIPP ghost axons and 2) ARCD of social memories. Indeed, in vivo lentiviral expression of the isolated PDE11A4-GAFB domain in hippocampal CA1 reverses the age-related accumulation of PDE11A4 in ghost axons, reverses ACRD of social transmission of food preference memory (STFP), and improves remote long-term memory for social odor recognition (SOR) without affecting memory for non-social odor recognition. In vitro studies suggest that disrupting homodimerization of PDE11A4 does not directly alter the catalytic activity of the enzyme but may reverse age-related decreases in cGMP by dispersing the accumulation of the enzyme independently of other intramolecular mechanisms previously established to disperse PDE11A4 (e.g., phosphorylation of PDE11A4 at serine 162). Although not wishing to be bound by any particular theory, altogether these data suggest that a biologic designed to disrupt PDE11A4 homodimerization may serve to ameliorate age-related deficits in hippocampal cyclic nucleotide signaling and subsequent ARCD of remote social memory.

Therefore, it is sought to determine if a biologic that decreases PDE11A4 homodimerization will be sufficient to reverse age-related accumulations of PDE11A4 in ghost axons and rescue ARCD of social memories.

### Methods

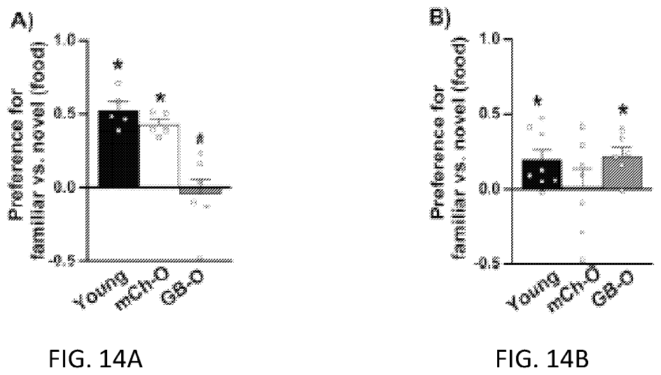

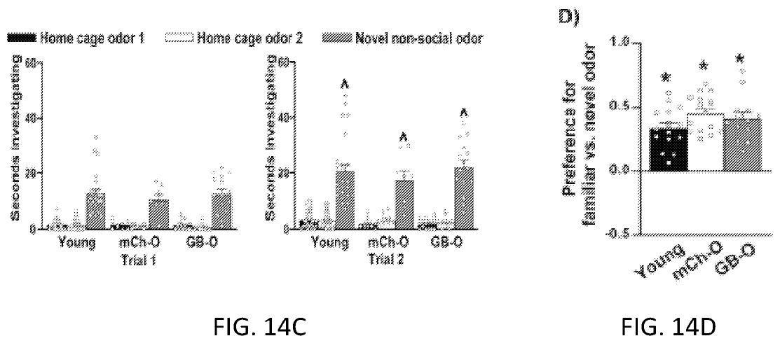

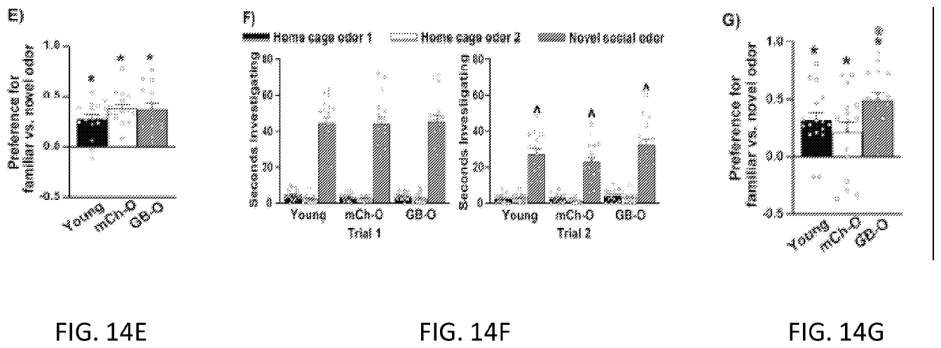

Subjects. C57BL6/J mice were originally obtained from Jackson Laboratory (Bar Harbor, ME) and the line was maintained at the University of South Carolina. The Pde11a mouse line obtained from Deltagen (San Mateo, CA) was maintained on a mixed C57BL6 background (99.8% multiple C57BL/6 substrains, 0.2% 129P2/OlaHsd). Pde11a mice were bred at the University of South Carolina in heterozygous (HT)×HT trio-matings. Same-sex wild-type (WT), heterozygous (HT), and knockout (KO) littermates were weaned and caged together to total 3-5 mice/cage. It is not believed litter effects are driving findings here due to each dataset reflecting multiple cohorts born and tested at different times. Further, a litter of mice normally contributes only 1-2 mice/genotype and parents contribute two litters at most to a cohort. While both males and females were used in experiments, analyze for sex effects (see figure legends for specific n's/sex/group/experiment). In these studies, young mice were defined as 2-6 months and old mice were defined as 18-22 months. “Young” mice included both young Pde11a WT mice surgerized alongside old Pde11a WT mice (i.e., receiving bilateral injections of mCherry lentivirus to the dorsal and ventral hippocampi) and unsurgerized young C57BL6/J mice that were used as an internal control for the assays (FIGS. 14A-14B). Since no obvious differences were found between groups of young surgerized Pde11a WT mice and young unsurgerized C57BL6/J mice, the data from these 2 subgroups were subsequently combined into a singular “young” (FIGS. 14C-14G). All mice used in experiments were generally healthy throughout the duration of testing. Gross pathology was not conducted but mice were routinely assessed by husbandry, veterinary, and laboratory staff. Mice with lethargy, altered gait, signs of malnutrition or dehydration, noticeable tumors>1 cm, were removed from study and euthanized.

The effects of healthy aging were studied, and therefore if upon brain dissection evidence was found of an anatomical abnormality (e.g., a pituitary tumor), the animals was excluded from the study (note: no occurrences in this study). A 12:12 light: dark cycle and ad lib access to food and water were provided. Experiments were conducted in accordance with the National Institutes of Health Guide for the Care and Use of Laboratory Animals (Pub 85-23, revised 1996) and were fully approved by the Institutional Animal Care and Use Committee of the University of South Carolina and the University of Maryland, Baltimore.

Tissue Collection: Mice were euthanized (during light cycle) via rapid cervical dislocation and brains were immediately collected and flash frozen on 2-methylbutane sitting on dry ice. Brain tissue was then stored at −80° C. until cryosectioning at 20 μm to verify viral expression.

Social Transmission of Food Preference. Subjects' access to food was restricted the two days prior to testing to one hour per day. The day prior to testing, all mice were placed in a clean home cage and given access to plain powdered chow packed into a glass jar. The following day, the designated “demonstrator mouse” was individually placed in a clean home cage and fed powdered chow flavored with a household spice (e.g. basil vs. ginger, thyme vs turmeric, mint vs cardamom, orange vs anise, basil vs thyme). After one hour, the “demonstrator mouse” was returned to the original home cage where the “observer” cage mates were allowed unrestricted access to the demonstrator for 15 minutes. It is during this time that the observers make an association between the social pheromones in the breath of the demonstrator and the non-social odor (household spice). Recent and remote long-term memory were assessed 24 hours or 7 days after training, respectively. At that time, the observer mice were individually placed in clean home cages and given access to two flavored/powdered chows for 1 hour. One flavored chow contained a novel spice and the other contained the spice that the demonstrator was given. The amount of food eaten was measured by an experimenter blind to treatment. All mice had to meet minimum inclusion criteria including eating at least 0.25 grams of food. Cohorts were able to be trained/tested at multiple time points using different spice combination to reduce the total number of mice used and we have shown that this does not confound interpretation of the data. Observer mice eating more food containing the familiar spice (i.e., the spice on their demonstrator's breath) versus the novel spiced food constituted memory (preference ratio: familiar-novel/familiar+novel).

Odor Recognition. Subjects were allowed to habituate to 1″ round wooden beads (Woodworks) for at least seven days prior to testing by placing several beads in the subjects' home cages. For social odor recognition (SOR), the wooden beads take up the scent of the cage of each strain used (e.g., C57BL/6J Jax #000664, BALB/cJ Jax #000651, 129S6/SvEv Taconic #129SVE) and are used as the social odor in testing. For non-social odor recognition (NSOR), the wooden beads were placed in a bag containing bedding saturated with a household spice (e.g., marjoram, cumin, etc.) for at least 7 days. Training for SOR and NSOR consisted of a habituation trial with 3 beads from the subject's home cage, followed by two training trials that included 2 home-cage beads and 1 novel-scented bead. Recent and remote long-term memory were assessed 24 hours or 7 days after training, respectively. During SOR, mice were tested with one home cage bead, one bead from the trained donor strain (familiar), and one bead from a second donor strain (novel). During NSOR, mice were tested with only two beads, one scented with the training spice and one a novel spice. The designation of which scent was “novel” within a given testing trial and the location of the novel scent (i.e., left versus right) was counterbalanced across subjects. Mice were given two minutes to investigate the beads and the amount of time spent on each was manually scored by an experimenter blind to treatment and bead. It was previously determined that infusion of even a negative control lentivirus into the hippocampus reverses the recent long-term memory impairment observed in Pde11a KO mice 24 hours after training. Therefore, 24-hour memory following injection of the isolated GAF-B domain was not tested as the results would not be interpretable. All mice met minimum inclusion criteria of spending a minimum of 3 seconds in total sniffing the beads. Spending more time investigating the novel vs familiar scent constituted memory (preference ratio: novel-familiar/novel+familiar).

Stereotaxic Surgeries. Stereotaxic surgeries and injections were performed using a NeuroStar motorized stereotaxic, drill, and injection robot (Tubingen, Germany). Mice were anesthetized with a steady flow of oxygen and isoflurane. The mice were induced at 3% isoflurane and maintained at 1-1.5%. Lack of reflexes was verified and the scalp was then shaved and cleaned with betadine. A small incision was made in the scalp and the skull was cleared with sterile saline. Cotton swabs were again used to visualize the skull and locate Bregma. Using the robotic drill, small holes were made above the dorsal and ventral hippocampi as per the following coordinates relative to Bregma: dCA1 AP, −1.7, dCA1 ML, +/−1.6, vCA1 AP, −3.5, vCA1 ML, +/−3.0. At a speed of 10 mm/sec, a Hamilton syringe (custom needle #7804-04: 26s gauge, 1″ length, 25 degree bevel) was then then placed to the following depths relative to Bregma: dCA1 DV, −1.3, vCA1 DV, −4.4. After a thirty second pause following the needle movement, the injection robot was used to inject 2 μl of lentivirus at 0.167 μl/minute. Following injection completion, the experimenter waited two minutes to allow the lentivirus to diffuse away from the needle and the needle was raised at the same speed. After all injections were complete, pronged tweezers were used to close the scalp and secured using glutures. Buprenorphine in sterile saline at a dose of 0.1 mg/kg was injected IP for pain management. For recovery, the mouse was placed on a warm Deltaphase pad and allowed to recover until moving normally and posturing upright. Mice were allowed at least 2 weeks of recovery in grouped home cages prior to behavioral testing.

A lentivirus carrying an mCherry-tagged PDE11A4-GAFB served to disrupt PDE11A4 homodimerization, while an mCherry-only virus was used as a negative control. A lentiviral construct was used here in order to compare to previous studies examining the effects of overexpressing PDE11A4 in vivo, which required the use of a lentiviral cassette due to the large size of PDE11A4. The viruses were made on an “SPW” backbone that drives expression using the phosphoglycerate kinase 1 (PGK) promoter, which in theory is a ubiquitous promoter and yet is taken up preferentially by neurons. It was previously shown that the isolated GAF-B construct disrupts PDE11A4 homodimerization by binding to PDE11A4 and triggering proteolytic degradation. For reasons that are not well understood, the GAF-B construct degrades PDE11A4 more significantly in the membrane versus cytosolic fractions. The lentiviruses were prepared and diluted in 0.2 M sucrose/42 mM NaCl/0.84 mM KCl/2.5 mM Na2HPO4/0.46 mM KH2PO4/0.35 mM EDTA and the original titers were as follows: mCherry, 7.37×10E10/mL; GAF-B, 1.82×10E10/ml. Pilot studies using wide-field fluorescent microscopy determined diluting the mCherry-only virus to one-third the original concentration equated comparable mCherry expression between viruses, and so this concentration was used in experiments. While high viral expression was found throughout CA1 of hippocampus in all mice, a subset of mice exhibited some viral expression in dentate gyrus, CA2, and/or CA3. This ectopic viral expression does not appear to affect the results given that 1) it is found in both the mCherry-only and mCherry-GAFB groups and 2) PDE11A4 expression does not emanate from the dentate, CA2 nor CA3.

It was found that cells in proximal CA1 (closer to CA3) relative to distal CA1 (closer to subiculum) more readily took up the virus, which mirrors the preferential distribution pattern of endogenous PDE11A4 across CA1. Importantly, no gross cellular toxicity or morphological damage was found with either virus, and animals were healthy following surgery. The accuracy of the injection and expression of the viral construct was verified by direct visualization of raw florescence and/or following immunofluorescence (see below).

Immunofluorescence. A cryostat was used to section fresh-frozen mouse brains at 20 μm. Sections were thaw-mounted onto slides and dried briefly at room temperature before storing at −80° C. until processing. The slides were fixed in 4% paraformaldehyde in phosphate-buffered saline (PBS) for 20 minutes. After fixation, 3×10 minute washes with PBS and 3×10 minute washes with PBT (phosphate-buffered saline/0.4% BSA/0.3% Triton-X 100) were performed to reduce background. Primary antibodies were combined in a tube with PBT at validated and optimized concentrations: PDE11A4 (Aves custom PDE11A4 #1 at 1:10,000; Fabgennix PPD11A-140AP at 1:1000; Fabgennix PPD11A-150AP at 1:500) and mCherry (ThermoFisher #PA534974 at 1:1000; Invitrogen #M11217 at 1:500; PhosphoSolutions #1203-mCherry at 1:10,000). Multiple PDE11A antibodies were utilized to discern diffuse expression versus accumulations of PDE11A4. While the PDE11A4 #1 antibody labels all PDE11A4 and, thus, detects both diffusely localized and accumulated PDE11A, the PDE11A4-140 and 150 antibodies specifically labels PDE11A4 phosphorylated at serines 117 and/or 124 and, thus, only detects PDE11A4 accumulated in ghost axons. To limit non-specific labeling, mCherry antibodies hosted in a rodent species (Invitrogen and PhosphoSolutions), were pretreated with anti-Mouse FabFragments (0.15 mg/ml; Jackson Immunoresearch #715-007-003) in PBS for 2 hours, followed by 3×10 minute washes in PBT prior to adding primary antibody. Primary antibody solution was added over brain sections and the slides were kept level at 4° C. overnight. Primary antibodies was removed using 4×10 minute washes in PBT. For optimal labeling, PDE11A4 secondary antibody (Alexafluor 488 AffiniPure Donkey Anti-Chicken, 1:1000, Jackson Immunoresearch #703-545-155) was applied first to the slides for 90 min at room temperature. The secondary was washed off using 3×10 minute washes with PBT. Next, the mCherry secondary (Alexafluor 594 AffiniPure species-specific, 1:1000, Jackson Immunoresearch) was repeated for the same time and conditions. Finally, 3×10 minute washes with PBT were used to clear the slides of any remaining secondary and the slides were briefly dip-rinsed in PBS to remove Triton. The slides were wiped dry along back, sides, and edges and mounted using DAPI Fluoromount-G (Southern Biotech, #0100-20). PDE11A4-filled structures were quantified by an experimenter blind to treatment with images captured using Leica Application Suite (LASX) software and a Leica DM5000 B florescent microscope. Brightness, histogram stretch, and/or contrast of images was adjusted for graphical clarity.

Cell culture and transfections. COS-1 (male line), HEK293T (female line), and HT-22 (sex undefined) cell culture and transfection were performed as previously described [1]. While kept in t-75 flasks, cells were grown in Dulbecco's Modified Eagle Medium (DMEM) (GIBCO; Gaithersburg, MD, USA), 10% fetal bovine serum (FBS) (GE Healthcare Life Sciences; Logan, UT, USA), and 1% Penicillin/Streptomycin (P/S) (Mediatech, a Corning subsidary; Manassas, VA, USA). Cells were incubated at 37° C./5% CO₂ and passaged using TrypLE Express (GIBCO; Gaithersburg, MD, USA) as a dissociation agent once 70-90% confluent. One day before transfection began, cells were plated along with DMEM+FBS+P/S in either 24-well plates for imaging or 100 mm dishes for biochemistry. The day of transfection, Optimem (GIBCO) replaced the media. According to manufacturer protocol (ratio of 3.75 μg DNA plus 10 μL lipofectamine per 10 mLs of media), cells were transfected with Lipofectamine 2000 (Invitrogen; Carlsbad, CA). ~19 hours post-transfection (PT), the Optimem/Lipofectamine solution was removed and replaced with DMEM+FBS+P/S. Normally, cells grew for five hours before sample processing, which meant they were harvested about 24 hours following transfection. Additionally, cells were sporadically tested for mycoplasma, with negative results always obtained. For assessment of subcellular trafficking, paraformaldehyde (4%) in PBS was used to fix the cells for fifteen minutes, after which they were kept in PBS until imaging. Images were captured using NIS-Elements BR-2.3 (Nikon, Tokyo Japan) on a CoolSNAP EZ CCD camera (Photometrics, Tucson AZ) mounted on an inverted Leica (Wetzlar DE) DMIL microscope with a Fluotar 10×/0.3 ∞/1.2 objective.

All images pertaining to an experiment were quantified by an experimenter blind to treatment using the same computer within the same position in the room, the same lighting conditions, and the same percent zoom. Images were loaded onto a gridded template to facilitate keeping track of count locations within the image, and an experimenter scored each image box by box, with cells along the top and left edges of the image as a whole not included to follow stereological best practices. Images were quantified in a counterbalanced manner such that 1 picture from each condition was evaluated before moving onto a 2nd image from that condition. The experimenter classified cells as exhibiting either cytosolic-only labeling or punctate labeling (with or without cytosolic labeling present), with data expressed as the % of the total number of labeled cells that exhibited punctate labeling.

Biochemical Fractionation and Western Blotting. Biochemical fractionation was performed to obtain cytosolic and soluble membrane fractions. Cell were mixed with ice cold fractionation buffer (20 mM Tris-HCl, pH 7.5; 2 mM MgCl₂; Thermo Pierce Scientific phosphatase tablet #A32959 and protease inhibitor 3 #P0044) and sonicated. First, a low-speed spin (1000×g) removed cellular debris and the supernatant from this spin was transferred to a new tube. Next, a high speed spin (89,000×g) was performed to obtain the membrane (pellet) and cytosolic (supernatant) proteins. After being suspended in fractionation buffer with 0.5% Triton-X 100 to solubilize the protein, the membrane pellet was sonicated and subjected to a high-speed spin (60,000×g). The supernatant containing all membrane proteins was then placed in a new tube. Samples were nutated for at 4° C. for 30 minutes. A second high-speed spin (60,000×g) was done for 30 minutes to separate the soluble membrane (supernatant) from the insoluble membrane (pellet). The soluble membrane sample was then transferred to a clean tube and used for western blot (see below). A DC Protein Assay kit (Bio-Rad; Hercules, CA, USA) was used to determine protein concentrations by which total protein was equalized across samples. Samples were stored at −80° C. until used in Western blotting. For Western blotting, 10 μg of protein was loaded onto 12% NuPAGE Bis-Tris gels (Invitrogen, Waltham MA) and run at 180 volts. After about one hour, the proteins remaining in the gels were transferred onto 0.45 μm nitrocellulose membrane using 100 mA for two hours. Following transfer, tris-buffered saline with 0.1% tween20 (TBS-T) was used to wash membranes. Membrane were cut into multiple strips to probe for multiple antibodies if needed. The membranes were blocked using either 5% milk or Superblock (PBS) Blocking Buffer (ThermoFisher, Cat #37515). Primary antibody was applied overnight at 4° C. for PDE11A (Fabgennix PD11-101 at 1:500). The following day, membranes were washed with TBS-T (4×10 minutes). Secondary antibody (Jackson Immunoresearch Anti-Rabbit, 111-035-144; 1:10 000), was applied at room temperature for one hour. Finally, the membrane was washed in TBS-T (3×15 minutes). Chemiluminescence (SuperSignal West Pico Chemilumiscent Substrate; ThermoScientific, Waltham MA) was captured using film and multiple exposures were taken to ensure densities were within the linear range of the film. ImageJ was used to quantify optical densities. Each blot was normalized to a control condition (e.g., WT) to account for any technical variables (film exposure, antibody signal-noise, variance in chemiluminescence, etc.).

PDE assay. cAMP- and cGMP-PDE catalytic activity were measured. The assay was validated in vitro using HT-22 cells (a mouse hippocampal cell line). Buffer containing 20 mM Tris-HCl and 10 mM MgCl₂ was used to harvest cells and kept on ice until ready to use. PDE activity was measured using 50 μl of sample and 50 μL of [3H] cAMP (Perkin Elmer, NET275) or cGMP (Perkin Elmer, NET337) and incubated for 10 minutes. After incubation, 0.1M HCl was added to quench the reaction, followed by 0.1M Tris to neutralize the reaction. 3.75 mg/mL snake venom (Crotalus atrox, Sigma V-7000) was then added to complete the reaction and the mixture was incubated at 37° C. for 10 minutes. Samples were put into 5′polystyrene chromatography columns with coarse filters (Evergreen, 208-3383-060) containing DEAE Sephadex A-25 resin (VWR, 95055-928). The columns were equilibrated in high salt buffer (20 mM Tris-HCL, 0.1% sodium azide, and 0.5 M NaCl) and low salt buffer (20 mM Tris-HCL and 0.1% sodium azide). The reactions were then run down the equilibrated columns. Following four washes with 0.5 ml of low salt buffer, 4 ml of Ultima Gold XR scintillation fluid (Fisher, 50-905-0519) was added to the eluate and mixed thoroughly. A Beckman-Coulter liquid scintillation counter Beckman LS 6000) was used to read counts per minute (CPM). As an assay control, two reactions free of sample lysate were ran in parallel to account for background activity and could then be subtracted from the sample CPMs. Total protein levels were quantified using the DC Protein Assay Kit (Bio-Rad, Hercules, CA) as described above, and CPMs were then normalized to the total amount of protein in each sample.

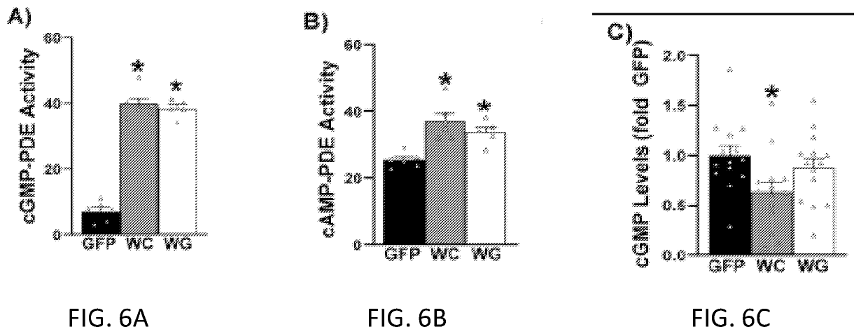

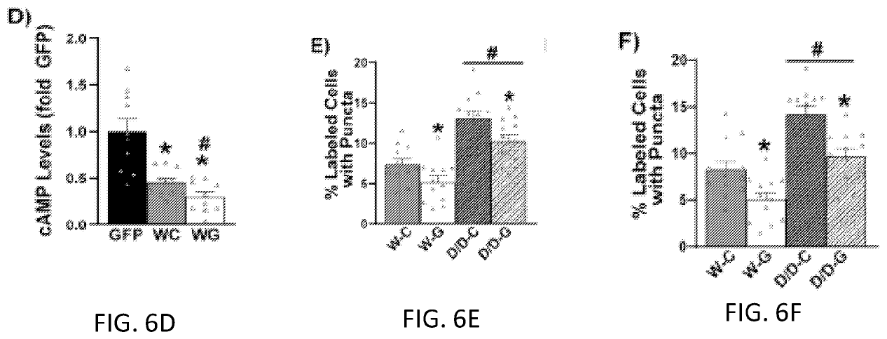

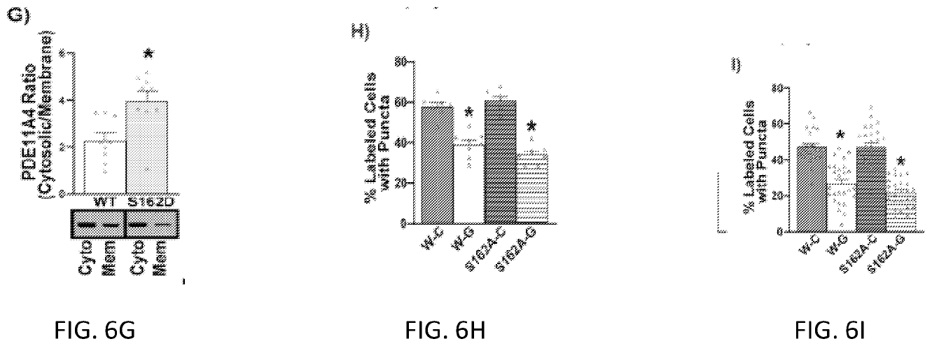

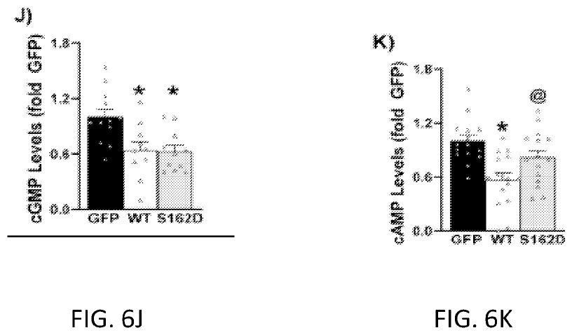

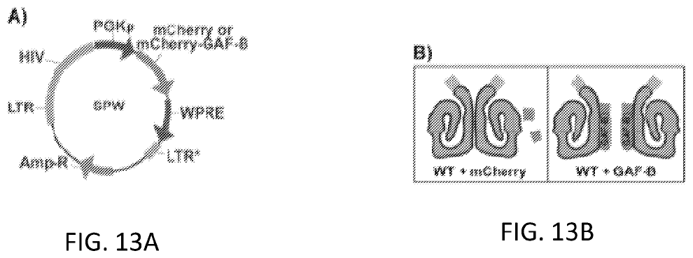

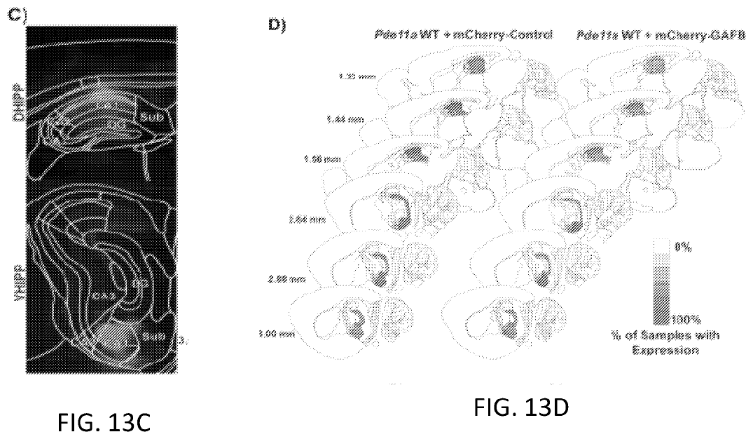

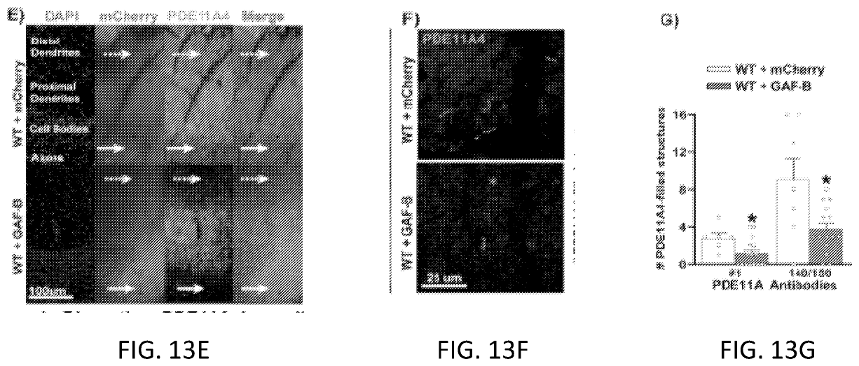

Data Analysis. Data was collected by investigators blind to treatment and experiments were designed to counterbalance technical/biological variables. Outliers more than 2 standard deviations away from the mean were removed prior to analyses. Outliers removed/total n: FIG. 13G, 1/23; FIG. 14A, 2/18; FIG. 14B, 1/22; FIG. 14E, 4/49; FIG. 6A, 1/18; FIG. 6B, 1/18; FIG. 6C, 3/45; FIG. 6G, 2/20; FIG. 6I, 9/112; FIG. 6J, 3/36; FIG. 6K, 3/36. Data were analyzed for effect of genotype, behavioral measure (e.g., bead or food), and treatment. Parametric statistical analyses were run on SigmaPlot 11.2 (San Jose, CA, USA) including ANOVA (F), Student's t-test (t), and one-sample t-test (t) when datasets met assumptions of normality (Shapiro-Wilk test) and equal variance (Levene's test). To offset the possibility of a Type I error associated with multiple comparisons, a false-rate discovery (FDR) correction was applied to P-values or one-sample t-tests within an experiment. If analyses failed normality and/or equal variance, nonparametric Kruskal-Wallis ANOVA (H) or Mann-Whitney rank sum test (T) were used instead. Student-Newman-Keuls or Dunn's test were performed for Post hoc analyses. Significance was defined as P<0.05.

### Results

Disrupting PDE11A4 homodimerization selectively decreases PDE11A4 expression in a compartment-specific manner and is sufficient to decrease PDE11A4 accumulations in ghost axons that occur with aging. It was previously found that ventral hippocampal PDE11A4 expression increases with age in both mice and humans and that these age-related increases accumulate specifically in the membrane compartment and within filamentous structures termed “ghost axons”. Additionally, it was found in vitro that disrupting PDE11A4 homodimerization by expressing an isolated GAF-B binding domain that acts as a negative sink (FIG. 13B) leads to proteolytic degradation specifically of membrane-bound PDE11A4 and reduces the accumulation of PDE11A4 into punctate structures. Therefore, the studies sought to determine if disrupting PDE11A homodimerization in vivo would be sufficient to reverse age-related increases in PDE11A4 expression and accumulation. To do this, lentiviruses were used (FIG. 13A) containing either mCherry alone (i.e., negative control) or an mCherry-tagged isolated GAF-B domain that disrupts PDE11A4 homodimerization (FIG. 13B). These lentiviruses were stereotactically injected bilaterally into the CA1 field of dorsal and ventral hippocampi of old Pde11a WT mice, since this is the field where PDE11A4 regulates social learning and memory. All mice demonstrated mCherry signal in dorsal and ventral CA1, with a subset of mice exhibiting expression in neighboring hippocampal sub-regions (e.g., dentate gyrus, CA3, CA2) that do not express PDE11A4 (FIG. 13D). While mCherry-treated mice exhibit a uniform pattern of PDE11A4 expression across stratum radiatum, stratum pyramidale and stratum oriens of CA1, GAF-B treated mice exhibit a compartment-specific decrease in PDE11A expression, with reduced expression in the distal segment of stratum radiatum and stratum oriens relative to stratum pyramidale (FIG. 13E). Consistent with the fact that the isolated GAF-B domain reduces PDE11A4 protein expression in the membrane compartment, it was found that disrupting homodimerization of PDE11A4 reduced age-related increases in so-called “PDE11A4 ghost axons” (i.e., filamentous structures harboring age-related accumulations of PDE11A4) (FIG. 13F). The ability of the GAF-B construct to reduce the accumulation of PDE11A4 in ghost axons was confirmed using two different PDE11A4 antibodies on two different sets of slides (FIG. 13G). Without wishing to be bound by any particular theory, all together these data suggest disruption of PDE11A homodimerization in vivo is sufficient to reverse age-related increases in PDE11A4 protein expression and ectopic accumulation.

Disrupting PDE11A4 homodimerization in the hippocampus of old mice is sufficient to reverse age-related decline of remote long-term social memory. It was found that while Pde11a WT mice suffer from ARCD of remote long-term social associative memories, Pde11a KO mice do not. Unfortunately, this protection of remote LTM 7 days after training comes at the expense of not being able to access recent LTM 24 hours after training. Therefore, it was determined if disrupting homodimerization of PDE11A4 using the isolated GAF-B domain is sufficient to induce a transient amnesia in old Pde11a WT mice that ultimately reverses ARCD of remote long-term social associative memories.

Social associative memory was measured using social transmission of food preference (STFP), an assay where mice form an association between a non-social odor (i.e., a household spice) and a social odor (i.e., pheromones in their cage mate's breath), with the memory of that association indicating a food with that scent is safe to eat. Mice treated with the GAF-B domain showed no recent LTM for STFP but did show remote LTM for STFP on par with that of young adult mice; whereas, mice treated with mCherry alone showed ARCD of remote LTM for STFP (FIGS. 14A-14B; Table 1). Importantly, no significant differences between the groups in terms of the total amount of food eaten was found (Table 2). To disentangle effects of the GAF-B construct on the non-social versus social components of the STFP assay, memory tests for odor recognition were examined. Old Pde11a WT mice treated with mCherry alone or mCherry-GAF-B spent the same amount of time sniffing the beads (Table 2) and learned equally well during training for non-social odor recognition (NSOR; FIG. 14C). They also demonstrated equally strong recent and remote LTM for NSOR (FIGS. 14D-DE; Table 1). Although not wishing to be bound by any particular theory, this result suggests that disrupting PDE11A4 homodimerization—like genetically deleting PDE11A—does not alter the ability to detect, learn about, or retrieve memories for recognizing non-social odors. In contrast, disrupting PDE11A4 homodimerization—again, like genetically deleting PDE11A—did significantly improve remote LTM for SOR memory (FIG. 14G; Table 1), despite having no effect on SOR learning (FIG. 14F) or total time sniffing (Table 2). Although not wishing to be bound by any particular theory, these data suggest that disrupting PDE11A4 homodimerization is sufficient to reverse ARCD of remote social memory.

TABLE 1
Preference ratios for each group calculated and subequently analyzed by one-sample t-test to determine
if their performance significantly differed from chance (i.e., 0) or determine whether or not
a given group exhibited a significant memory. The resulting P-values within an experiment were
then corrected for multiple comparisons using false discovery rate correction (FDR).

| Datasets | Young | mCh-O | GB-O |
|---|---|---|---|
| FIG. 2A | t(4) = 9.51, FDR-P < 0.001 | t(4) = 12.60, FDR-P < 0.001 | t(14) = 6.74, FDR-P < 0.001 |
| STFP 24 h LTM | | | |
| FIG. 2B | t(7) = 2.90, FDR-P = 0.036 | t(6) = 0.14, FDR-P = 0.896 | t(5) = 3.84, FDR-P = 0.036 |
| STFP 7 d LTM | | | |
| FIG. 2D | t(14) = 11.9, FDR-P < 0.001 | t(14) = 8.19, FDR-P < 0.001 | t(12) = 8.48, P < 0.001 |
| NSOR 24 h LTM | | | |
| FIG. 2E | t(14) = 9.75, FDR-P < 0.001 | t(14) = 9.45, FDR-P < 0.001 | t(14) = 8.04, FDR-P < 0.001 |
| NSOR 7 d LTM | | | |
| FIG. 2G | t(16) = 7.49, FDR-P < 0.001 | t(15) = 2.18, FDR-P = 0.046 | t(14) = 6.74, FDR-P < 0.001 |
| SOR 7 d LTM | | | |

TABLE 2
Amount of food (grams) eaten during STFP tests or amount of time spent invenstigating
(seconds) during SOR/NSOR tests by young controls and old mice treated
with mCherry (mCh-O) or GAF-B (GB-O) lentivirus during 24-hour and 7-day
long-term memory (LTM) tests (Data expressed as mean ± SEM).

| Datasets | Young | mCh-O | GB-O | *effect of group: |
|---|---|---|---|---|
| FIG. 2A | 0.96 ± 0.08 | 1.15 ± 0.11 | 1.23 ± 0.13 | F(2, 15) = 1.63, P = 0.23 |
| STFP 24 h LTM | | | | |
| FIG. 2B | 0.94 ± 0.12 | 0.93 ± 0.10 | 0.95 ± 0.05 | F(2, 19) = 0.01, P = 0.99 |
| STFP 7 d LTM | | | | |
| FIG. 2D | 37.06 ± 2.59 | 29.44 ± 2.90 | 29.73 ± 3.44 | F(2, 45) = 2.19, P = 0.124 |
| NSOR 24 h LTM | | | | |
| FIG. 2E | 33.47 ± 2.84 | 23.75 ± 2.98 | 26.50 ± 3.13 | F(2, 46) = 2.88, P = 0.067 |
| NSOR 7 d LTM | | | | |
| FIG. 2G | 52.77 ± 4.48 | 29.38 ± 2.77 | 28.67 ± 2.67 | H(2) = 16.22, P < 0.001 |
| SOR 7 d LTM | | | | |

*Data analyzed for effect of group by One-Way ANOVA (F) when data passed assumptions of normality and equal variance or by ANOVA on Ranks (H) when assumptions failed. Old mice spent less time sniffing during social odor recognition (SOR) than young mice. There were no effects of group on time spent investigating during non-social odor recognition (NSOR).

PDE11A4 homodimerization is an independent intramolecular mechanism that regulates PDE11A4 trafficking and functioning. As noted above, disrupting PDE11A4 homodimerization significantly changes the subcellular compartmentalization of the enzyme. To better understand the functional consequences of disrupting PDE11A4 homodimerization, we measured PDE activity and cyclic nucleotide levels in cells transfected with GFP+mCherry (i.e., negative control), GFP-PDE11A4+mCherry, or GFP-PDE11A4+mCherry-GAF-B. Compared to the negative control, expression of PDE11A4 significantly increased cGMP- and cAMP-PDE activity in HT-22 cells as expected. This increase in PDE activity was not altered by disruption of PDE11A4 homodimerization (FIGS. 6A-6B), which is consistent with previous studies using purified PDE11A4 enzyme. Also as expected, expression of PDE11A4 in concert with mCherry decreased both cGMP and cAMP levels in COS1 cells relative to the negative control (FIGS. 6C-6D). Interestingly, disrupting homodimerization of PDE11A4 decreased PDE11A4 hydrolysis of cGMP while exacerbating PDE11A4 degradation of cAMP (FIG. 6C-6D). The ability of the GAF-B construct to alter cyclic nucleotide levels in absence of a direct effect on PDE11A4 enzymatic activity is likely due to changes in subcellular compartmentalization of PDE11A4 possibly shifting it from a CGMP-rich pool to a cAMP-rich pool. Importantly, disrupting homodimerization not only reduces the accumulation of WT PDE11A4, it also reduces back to WT levels the potentiated accumulation caused by the aging-related phosphomimic mutant S117D/S124D. Although not wishing to be bound by any particular theory, together these data suggest disruption of PDE11A4 homodimerization alters cyclic nucleotide signaling not by altering PDE11A4 catalytic activity directly, but rather by changing the subcellular localization of PDE11A4 protein (e.g., shifting from a cGMP-rich pool to a cAMP-rich pool).

It was next determined if the isolated GAF-B domain could reduce accumulation not only of WT PDE11A4 but also PDE11A4-S117D/S124D, which mimics the age-related increase in PDE11A4-pS117/pS124 that drives the punctate accumulation of PDE11A4 in the aged brain. Across multiple experiments and cell lines (i.e., HEK293T and HT-22), the isolated GAF-B domain reduced the punctate accumulation of both the WT and PDE11A4-S117D/S124D compared to the mCherry control (FIG. 6E-6F). Together, these data suggest that disrupting PDE11A4 homodimerization may reverse age-related decreases in cGMP signaling by counteracting the effect of age-related increases in PDE11A4-pS117/pS124.

PDE11A homodimerization does not require phosphorylation of S162. As described above, disrupting PDE11A4 homodimerization reduces the accumulation of PDE11A4 in punctate structures both in vivo (FIG. 13F) and in vitro (FIGS. 6E-6F). Since the ability of the isolated GAF-B domain to disperse PDE11A4 accumulations strongly resembles that of a phosphomimic mutation at S162 (i.e., S162D), it was sought to determine if disrupting homodimerization promoted phosphorylation of S162. Such a dispersing phenotype is also triggered by a phosphomimic mutation at S162 (i.e., S162D). As such, it was determined if disrupting PDE11A4 homodimerization may work by promoting phosphorylation of S162. Indeed, S162D changed the subcellular compartmentalization of PDE11A4 in a manner similar to that observed with the isolated GAF-B domain-namely, shifting PDE11A4 from the membrane to the cytosolic fraction (FIG. 6G). The isolated GAF-B domain was able to effectively reduce the accumulation of both WT PDE11A4 as well as the phosphoresistant PDE11A4-S162A, suggesting phosphorylation of S162 is not needed for the dispersing effect of the isolated GAF-B domain (FIG. 6H-6I). While not wishing be bound by any particular theory, this result suggests that disrupting homodimerization does not achieve effects by promoting phosphorylation of S162. Indeed, S162D differed substantially from GAF-B in terms of regulating cyclic nucleotide levels. It was found that S162D elicited quite different effects on cyclic nucleotide levels than were described above for the isolated GAF-B domain (FIGS. 6C-6D). Specifically, S162D did not alter PDE11A4 hydrolysis of cGMP (FIG. 6J) and appeared to reduce PDE11A4 hydrolysis of cAMP (FIG. 6K). Although not wishing to be bound by any particular theory, together these data suggest that homodimerization and pS162 are independent intramolecular mechanism that regulate PDE11A4 trafficking and function.

### Discussion

Previously, it was found that age-related increases in PDE11A4 occur specifically within the membrane compartment of the VHIPP and ectopically accumulate in filamentous structures termed “ghost axons” due to age-related increases in the phosphorylation of S117/S124. Here, it was shown that disrupting PDE11A4 homodimerization within hippocampal CA1 using a biologic encoding an isolated GAF-B domain reverses the age-related accumulation of PDE11A4 in ghost axons in vivo, reverses the accumulating effect of S117D/S124D in vitro, and reduces PDE11A4 expression in ventral CA1 in a compartment-specific manner (i.e., in distal dendrites and stratum oriens; FIGS. 13E-13G). Further, it was shown that disrupting PDE11A homodimerization is sufficient to reverse ARCD of remote social aLTMs—albeit at the expense of an inability to retrieve recent social LTMs (FIGS. 14A-14G).

Such a transient amnesia that ultimately produces stronger remote social LTMs is also observed with genetic deletion of Pde11a in young and old adult mice. Although disrupting PDE11A homodimerization does not alter catalytic activity of the enzyme, it changes the subcellular localization of PDE11A4 (FIGS. 6E-6F), which ultimately leads to higher cGMP levels and slightly lower cAMP levels (FIGS. 6C-6D). Although not wishing to be bound by any particular theory, this result suggests that disrupting PDE11A4 homodimerization shifts PDE11A from a cGMP-rich pool to a cAMP-rich pool, thereby alleviating age-related decreases in cGMP that are widely reported to occur in the aging hippocampus. Interestingly, phosphorylation of PDE11A4 at serine 162 (pS162) similarly reduces the accumulation of PDE11A4 in punctate structures and shifts PDE11A4 from the membrane to the cytosol (FIG. 6G) as does disruption of homodimerization. That said, it was shown herein that these two regulatory mechanisms clearly operate independently since disrupting homodimerization 1) reduces the accumulation of PDE11A4 even when phosphorylation of S162 was prevented using the S162A mutant (FIGS. 6H-6I) and 2) decreased cAMP levels (FIG. 6D) while mimicking phosphorylation of S162 increased cAMP levels (FIG. 6K). Although not wishing to be bound by any particular theory, together these data suggest that disrupting PDE11A4 homodimerization in vivo via expression of its isolated GAF-B domain is sufficient to reverse age-related increases in the ectopic accumulation of PDE11A4 within ghost axons and rescue ARCD of remote social aLTMs independently of phosphorylating S162.

Post-translational modifications and protein-protein interactions alter the subcellular localization and trafficking of PDE11A4 protein. The subcellular compartmentalization of PDEs are regulated by multiple mechanisms. The localization of the PDE2, PDE4, PDE5, PDE10, and PDE11 families is regulated in part by post-translational modifications. For instance, phosphorylation of PDE10A prevents membrane insertion by blocking palmitoylation. As opposed to direct membrane insertion, PDE11A4 is thought to associate with the membrane by binding to a macromolecular complex. The ability of PDE11A4 to interact with this macromolecular complex appears to be reduced when PDE11A4 homodimerization is disrupted or when S162 is phosphorylated (FIG. 6G), as both intramolecular signals cause PDE11A4 to shift from the membrane to the cytosol. Interestingly, this shift away from the membrane compartment following disruption of homodimerization or introduction of the S162D phophomimic mutation is accompanied by a reduction in the punctate accumulation of PDE11A4. Despite these similarities, however, the two regulatory mechanisms have opposing effects on cAMP levels (FIG. 6D vs. FIG. 6K). These opposing effects suggest PDE11A4 is being redistributed to differing cytosolic compartments in response to disrupted homodimerization versus phosphorylation of S162. Indeed, disrupting homodimerization acts independently of pS162 to reduce the punctate accumulation of PDE11A4 (FIGS. 6H-6I). Phosphorylation of PDE11A4 at serines 117 and 124 (pS117/pS124), on the other hand, increases expression and accumulation of PDE11A4 protein. PKA and PKG have been shown to phosphorylate S117 and S162 in vitro, and these same kinases are predicted to phosphorylate S124. Cyclic nucleotide levels controlled by PDE11A4 could then modulate PKA/PKG activity at S117, S124, and/or S162, thereby creating direct feedback/feedforward loop as has been described for other PDE families.

In addition to post-translational modification, protein-protein interactions regulate the subcellular localization of PDEs. Although not wishing to be bound by any particular theory, these results suggest that homodimerization is a key type of protein-protein interaction that regulates the compartmentalization of PDE11A4 both in vitro and in vivo. For example, the PDE11A4 sequence in BALB/cJ mice differs from that of C57BL/6J mice at a single amino acid that falls within the GAF-B domain—whereas BALB/cJ mice encode a threonine at amino acid 499, C57BL/6J mice encode an alanine. This A499T BALB/cJ mutation strengthens homodimerization, elevates protein expression, and increases the punctate accumulation of PDE11A4 both in vitro and in vivo. In contrast, disrupting homodimerization using the isolated GAF-B domain has the opposite effects, lowering protein expression of PDE11A4 due to increased proteolysis and reducing the punctate accumulation of PDE11A4 both in vivo and in vitro; FIGS. 13E-13G, 6E-6F and 6H-6I). Although not wishing to be bound by any particular theory, these studies suggest that age-related increases in PDE11A4 expression/accumulation may be prevented/reversed by reducing levels of PDE11A4 homodimerization. Indeed, it is shown here that disrupting PDE11A4 homodimerization in old WT mice was sufficient to reverse age-related accumulation of PDE11A4 in ghost axons (FIGS. 13F-13G) and ARCD of remote social aLTMs (FIG. 14B). These positive effects may be related to the fact that disrupting PDE11A4 homodimerization leads to increased cGMP levels (F FIG. 6C), which may counteract decreases in hippocampal cGMP levels that occur with age.

Disrupting PDE11A4 homodimerization as a therapeutic approach for treating ARCD. A hallmark pathology of the aging brain includes ectopic protein expression and accumulation in the brain, as well as the loss of cGMP signaling in the hippocampus. Much like hyperphosphorylated tau causes neurofibrillary tangles that lead to impaired cell communication, function, and death, it was found that phosphorylation of PDE11A4 at serines S117 and S124 increases PDE11A4 protein expression and ectopic accumulation in filamentous structures termed “ghost axons.” These age-related increases in PDE11A protein appear to cause ARCD of social memories via the cGMP-PKG, as opposed to CAMP-PKA, pathway. This is consistent with reports that aging and ARCD are associated with decreases in cGMP, but not cAMP, in the hippocampus, and that elevating cGMP levels elicits nootropic effects in the context of ARCD and neurodegenerative diseases. Therefore, it is particularly noteworthy that disrupting PDE11A4 homodimerization increases cGMP levels (FIGS. 14A-14G), possibly by removing PDE11A4 from hydrolyzing a key nanodomain of cGMP.

Although it is clear that age-related increases in PDE11A4 protein expression are deleterious to remote social memory, it is possible that the ectopic accumulation of PDE11A in ghost axons may actually serve as a protective mechanism to sequester and/or neutralize excess PDE11A4. An example of this type of sequestration is found with some PDE4A isoforms when they are bound by conformationally-altering catalytic inhibitors. Therefore, therapeutic targeting of PDE11A4 intended to reduce its ectopic accumulation may also need to promote clearance of PDE11A4 from the system. As noted above, disrupting PDE11A4 homodimerization significantly decreases the puctate accumulation of PDE11A4 and specifically promotes degradation of membrane-associated PDE11A4. The results described herein suggest that disruption of PDE11A4 homodimerization may be a sophisticated mechanism for therapeutic targeting of age-related increases in PDE11A4, by decreasing both its accumulation and clearing it from select compartments. The data described herein demonstrate that disruption of PDE11A4 homodimerization using a biologic-such as the biologic described herein-represents a novel therapeutic approach for treating ARCD, including ARCD of social memories.

### REFERENCES

1. Kelly M P. A Role for Phosphodiesterase 11A (PDE11A) in the Formation of Social Memories and the Stabilization of Mood. Advances in neurobiology. 2017; 17:201-30. doi: 10.1007/978-3-319-58811-7_8. PubMed PMID: 28956334; PubMed Central PMCID: PMC5652326.
2. Hegde S, Capell W R, Ibrahim B A, Klett J, Patel N S, Sougiannis A T, Kelly M P. Phosphodiesterase 11A (PDE11A), Enriched in Ventral Hippocampus Neurons, is Required for Consolidation of Social but not Nonsocial Memories in Mice. Neuropsychopharmacology: official publication of the American College of Neuropsychopharmacology. 2016; 41(12): 2920-31. doi: 10.1038/npp.2016.106. PubMed PMID: 27339393; PubMed Central PMCID: PMC5061884.
3. Abbott A. Cognition: The brain's decline. Nature. 2012; 492(7427): S4-5. doi: 10.1038/492S4a. PubMed PMID: 23222672.
4. Guerreiro R, Bras J. The age factor in Alzheimer's disease. Genome medicine. 2015; 7:106. doi: 10.1186/s13073-015-0232-5. PubMed PMID: 26482651; PubMed Central PMCID: PMC4617238.
5. Plassman B L, Langa K M, Fisher G G, Heeringa S G, Weir D R, Ofstedal M B, Burke J R, Hurd M D, Potter G G, Rodgers W L, Steffens D C, McArdle J J, Willis R J, Wallace R B. Prevalence of cognitive impairment without dementia in the United States. Annals of internal medicine. 2008; 148(6): 427-34. PubMed PMID: 18347351; PubMed Central PMCID: PMC2670458.
6 Bender A R, Naveh-Benjamin M, Raz N. Associative deficit in recognition memory in a lifespan sample of healthy adults. Psychology and aging. 2010; 25(4): 940-8. doi: 10.1037/a0020595. PubMed PMID: 20822256; PubMed Central PMCID: PMC3011045.
7. Yuasa K, Ohgaru T, Asahina M, Omori K. Identification of rat cyclic nucleotide phosphodiesterase 11A (PDE11A): comparison of rat and human PDE11A splicing variants. European journal of biochemistry/FEBS. 2001; 268(16): 4440-8. PubMed PMID: 11502204.
8. Yuasa K, Kanoh Y, Okumura K, Omori K. Genomic organization of the human phosphodiesterase PDE11A gene. Evolutionary relatedness with other PDEs containing GAF domains. European journal of biochemistry/FEBS. 2001; 268(1): 168-78. PubMed PMID: 11121118.
9. Wong M L, Whelan F, Deloukas P, Whittaker P, Delgado M, Cantor R M, McCann S M, Licinio J, Wong M L, Whelan F, Deloukas P, Whittaker P, Delgado M, Cantor R M, McCann S M, Licinio J. Phosphodiesterase genes are associated with susceptibility to major depression and antidepressant treatment response. Proceedings of the National Academy of Sciences of the United States of America. 2006; 103(41): 15124-9.
10. Luo H R, Wu G S, Dong C, Arcos-Burgos M, Ribeiro L, Licinio J, Wong M L. Association of PDE11A global haplotype with major depression and antidepressant drug response. Neuropsychiatric disease and treatment. 2009; 5:163-70. PubMed PMID: 19557111; PubMed Central PMCID: PMC2695232.
11. Cabanero M, Laje G, Detera-Wadleigh S, McMahon F J. Association study of phosphodiesterase genes in the Sequenced Treatment Alternatives to Relieve Depression sample. Pharmacogenetics and genomics. 2009; 19(3): 235-8. doi: 10.1097/FPC.0b013e328320a3e2. PubMed PMID: 19214142; PubMed Central PMCID: PMC2819180.
12. Coon H, Darlington T, Pimentel R, Smith K R, Huff C D, Hu H, Jerominski L, Hansen J, Klein M, Callor W B, Byrd J, Bakian A, Crowell S E, McMahon W M, Rajamanickam V, Camp N J, McGlade E, Yurgelun-Todd D, Grey T, Gray D. Genetic risk factors in two Utah pedigrees at high risk for suicide. Translational psychiatry. 2013; 3: e325. doi: 10.1038/tp.2013.100. PubMed PMID: 24252905; PubMed Central PMCID: PMC3849959.
13. Perlis R H, Fijal B, Dharia S, Heinloth A N, Houston J P. Failure to replicate genetic associations with antidepressant treatment response in duloxetine-treated patients. Biological psychiatry. 2010; 67(11): 1110-3. doi: 10.1016/j.biopsych.2009.12.010. PubMed PMID: 20110084.
14. Couzin J. Science and commerce. Gene tests for psychiatric risk polarize researchers. Science. 2008; 319(5861): 274-7. doi: 10.1126/science.319.5861.274. PubMed PMID: 18202268.
15. Kelsoe J, inventor; THE REGENTS OF THE UNIVERSITY OF CALIFORNIA (Oakland, CA), assignee. METHOD TO PREDICT RESPONSE TO TREATMENT FOR PSYCHIATRIC ILLNESSES. USA 2010.
16. Mertens J, Wang Q W, Kim Y, Yu D X, Pham S, Yang B, Zheng Y, Diffenderfer K E, Zhang J, Soltani S, Eames T, Schafer S T, Boyer L, Marchetto M C, Nurnberger J I, Calabrese J R, Odegaard K J, McCarthy M J, Zandi P P, Alda M, Nievergelt C M, Pharmacogenomics of Bipolar Disorder S, Mi S, Brennand K J, Kelsoe J R, Gage F H, Yao J. Differential responses to lithium in hyperexcitable neurons from patients with bipolar disorder. Nature. 2015; 527(7576): 95-9. doi: 10.1038/nature15526. PubMed PMID: 26524527; PubMed Central PMCID: PMC4742055.
17. Rizzo L B, Costa L G, Mansur R B, Swardfager W, Belangero S I, Grassi-Oliveira R, McIntyre R S, Bauer M E, Brietzke E. The theory of bipolar disorder as an illness of accelerated aging: implications for clinical care and research. Neuroscience and biobehavioral reviews. 2014; 42:157-69. doi: 10.1016/j.neubiorev.2014.02.004. PubMed PMID: 24548785.
18. Kinser P A, Lyon D E. Major depressive disorder and measures of cellular aging: an integrative review. Nursing research and practice. 2013; 2013:469070. doi: 10.1155/2013/469070. PubMed PMID: 23691300; PubMed Central PMCID: PMC3649747.
19. Kelly M P, Logue S F, Brennan J, Day J P, Lakkaraju S, Jiang L, Zhong X, Tam M, Sukoff Rizzo S J, Platt B J, Dwyer J M, Neal S, Pulito V L, Agostino M J, Grauer S M, Navarra R L, Kelley C, Cornery T A, Murrills R J, Houslay M D, Brandon N J. Phosphodiesterase 11A in brain is enriched in ventral hippocampus and deletion causes psychiatric disease-related phenotypes. Proceedings of the National Academy of Sciences of the United States of America. 2010; 107(18): 8457-62. doi: 10.1073/pnas. 1000730107. PubMed PMID: 20404172; PubMed Central PMCID: PMC2889541.
20. Kelly M P. Does phosphodiesterase 11A (PDE11A) hold promise as a future therapeutic target? Current pharmaceutical design. 2015; 21(3): 389-416. PubMed PMID: 25159071.
21. Pathak G, Agostino M J, Bishara K, Capell W R, Fisher J L, Hegde S, Ibrahim B A, Pilarzyk K, Sabin C, Tuczkewycz T, Wilson S, Kelly M P. PDE11A negatively regulates lithium responsivity. Molecular psychiatry. 2017; 22(12): 1714-24. doi: 10.1038/mp.2016.155. PubMed PMID: 27646265; PubMed Central PMCID: PMC5359083.
22. Kelly M P. PDE11A. In: Choi S, editor. Encyclopedia of Signaling Molecules. Cham: Springer International Publishing; 2018. p. 3804-26.
23. Kelly M P, Adamowicz W, Bove S, Hartman A J, Mariga A, Pathak G, Reinhart V, Romegialli A, Kleiman R J. Select 3′,5′-cyclic nucleotide phosphodiesterases exhibit altered expression in the aged rodent brain. Cellular signalling. 2014; 26(2): 383-97. doi: 10.1016/j.cellsig.2013.10.007. PubMed PMID: 24184653.
24. Jager R, Russwurm C, Schwede F, Genieser H G, Koesling D, Russwurm M. Activation of PDE10 and PDE11 phosphodiesterases. The Journal of biological chemistry. 2012; 287(2): 1210-9. doi: 10.1074/jbc.M111.263806. PubMed PMID: 22105073; PubMed Central PMCID: PMC3256904.
25. Hegde S, Ji H, Oliver D, Patel N S, Poupore N, Shtutman M, Kelly M P. PDE11A regulates social behaviors and is a key mechanism by which social experience sculpts the brain. Neuroscience. 2016; 335:151-69. doi: 10.1016/j.neuroscience.2016.08.019. PubMed PMID: 27544407; PubMed Central PMCID: PMC5031549.
26. Zhang C, Cheng Y, Wang H, Wang C, Wilson S P, Xu J, Zhang H T. RNA interference-mediated knockdown of long-form phosphodiesterase-4D (PDE4D) enzyme reverses amyloid-beta42-induced memory deficits in mice. Journal of Alzheimer's disease: JAD. 2014; 38(2): 269-80. doi: 10.3233/JAD-122236. PubMed PMID: 23948935.
27. Bonkale W L, Cowburn R F, Ohm T G, Bogdanovic N, Fastbom J. A quantitative autoradiographic study of └3H┘cAMP binding to cytosolic and particulate protein kinase A in post-mortem brain staged for Alzheimer's disease neurofibrillary changes and amyloid deposits. Brain research. 1999; 818(2): 383-96. PubMed PMID: 10082824.
28. Titus D J, Furones C, Kang Y, Atkins C M. Age-dependent alterations in cAMP signaling contribute to synaptic plasticity deficits following traumatic brain injury. Neuroscience. 2013; 231:182-94. doi: 10.1016/j.neuroscience.2012.12.002. PubMed PMID: 23238576; PubMed Central PMCID: PMC3558555.
29. Countryman R A, Gold P E. Rapid forgetting of social transmission of food preferences in aged rats: relationship to hippocampal CREB activation. Learning & memory. 2007; 14(5): 350-8. doi: 10.1101/lm.524907. PubMed PMID: 17522026; PubMed Central PMCID: PMC1876759.
30. Sha L, Macintyre L, Machell J A, Kelly M P, Porteous D J, Brandon N J, Muir W J, Blackwood D H, Watson D G, Clapcote S J, Pickard B S. Transcriptional regulation of neurodevelopmental and metabolic pathways by NPAS3. Molecular psychiatry. 2012; 17(3): 267-79. Epub 2011 Jun. 29. doi: 10.1038/mp.2011.73. PubMed PMID: 21709683.
31. Maxwell C R, Liang Y, Kelly M P, Kanes S J, Abel T, Siegel S J. Mice expressing constitutively active Gsalpha exhibit stimulus encoding deficits similar to those observed in schizophrenia patients 2006; 141(3): 1257-64.
32. Kelly M P, Stein J M, Vecsey C G, Favilla C, Yang X, Bizily S F, Esposito M F, Wand G, Kanes S J, Abel T. Developmental etiology for neuroanatomical and cognitive deficits in mice overexpressing Galphas, a G-protein subunit genetically linked to schizophrenia. Molecular psychiatry. 2009; 14(4): 398-415, 347. Epub 2008 Nov. 26. doi: 10.1038/mp.2008.124. PubMed PMID: 19030002; PubMed Central PMCID: PMC3312743.
33. Kelly M P, Isiegas C, Cheung Y F, Tokarczyk J, Yang X, Esposito M F, Rapoport D A, Fabian S A, Siegel S J, Wand G, Houslay M D, Kanes S J, Abel T. Constitutive activation of Galphas within forebrain neurons causes deficits in sensorimotor gating because of PKA-dependent decreases in cAMP. Neuropsychopharmacology: official publication of the American College of Neuropsychopharmacology. 2007; 32(3): 577-88. doi: 10.1038/sj.npp. 1301099. PubMed PMID: 16738544; PubMed Central PMCID: PMC3303872.
34. Kelly M P, Cheung Y F, Favilla C, Siegel S J, Kanes S J, Houslay M D, Abel T. Constitutive activation of the G-protein subunit Galphas within forebrain neurons causes PKA-dependent alterations in fear conditioning and cortical Arc mRNA expression. Learning & memory. 2008; 15(2): 75-83. doi: 10.1101/lm.723708. PubMed PMID: 18230676; PubMed Central PMCID: PMC2216679.
35. Gould T J, Bizily S P, Tokarczyk J, Kelly M P, Siegel S J, Kanes S J, Abel T. Sensorimotor gating deficits in transgenic mice expressing a constitutively active form of Gs-alpha. Neuropsychopharmacology: official publication of the American College of Neuropsychopharmacology. 2004; 29(3): 494-501. Epub 2003 Dec. 25. doi: 10.1038/sj.npp 1300309. PubMed PMID: 14694347; PubMed Central PMCID: PMC3348581.
36. Favilla C, Abel T, Kelly M P. Chronic Galphas signaling in the striatum increases anxiety-related behaviors independent of developmental effects. The Journal of neuroscience: the official journal of the Society for Neuroscience. 2008; 28(51): 13952-6. Epub 2008 Dec. 19. doi: 10.1523/JNEUROSCI.4986-08.2008. PubMed PMID: 19091983; PubMed Central PMCID: PMC2688724.
37. Bourtchouladze R, Patterson S L, Kelly M P, Kreibich A, Kandel E R, Abel T. Chronically increased Gsalpha signaling disrupts associative and spatial learning. Learning & memory. 2006; 13(6): 745-52. Epub 2006 Dec. 5. doi: 10.1101/lm.354106. PubMed PMID: 17142304; PubMed Central PMCID: PMC1783628.
38. Wang Q, Charych E I, Pulito V L, Lee J B, Graziane N M, Crozier R A, Revilla-Sanchez R, Kelly M P, Dunlop A J, Murdoch H, Taylor N, Xie Y, Pausch M, Hayashi-Takagi A, Ishizuka K, Seshadri S, Bates B, Kariya K, Sawa A, Weinberg R J, Moss S J, Houslay M D, Yan Z, Brandon N J. The psychiatric disease risk factors DISC1 and TNIK interact to regulate synapse composition and function. Molecular psychiatry. 2011; 16(10): 1006-23. Epub 2010 Sep. 15. doi: 10.1038/mp.2010.87. PubMed PMID: 20838393; PubMed Central PMCID: PMC3176992.
39. Gross-Langenhoff M, Stenzl A, Altenberend F, Schultz A, Schultz J E. The properties of phosphodiesterase 11A4 GAF domains are regulated by modifications in its N-terminal domain. The FEBS journal. 2008; 275(8): 1643-50. doi: 10.1111/j.1742-4658.2008.06319.x. PubMed PMID: 18312413.
40. Weeks J L, 2nd, Zoraghi R, Francis S H, Corbin J D. N-Terminal domain of phosphodiesterase-11A4 (PDE11A4) decreases affinity of the catalytic site for substrates and tadalafil, and is involved in oligomerization. Biochemistry. 2007; 46(36): 10353-64. doi: 10.1021/bi7009629. PubMed PMID: 17696499.
41. Weeks J L, Zoraghi R, Beasley A, Sekhar K R, Francis S H, Corbin J D. High biochemical selectivity of tadalafil, sildenafil and vardenafil for human phosphodiesterase 5A1 (PDE5) over PDE11A4 suggests the absence of PDE11A4 cross-reaction in patients. International journal of impotence research. 2005; 17(1): 5-9. doi: 10.1038/sj.ijir.3901283. PubMed PMID: 15538396.
42. Kelly M P, Isiegas C, Cheung Y F, Tokarczyk J, Yang X, Esposito M F, Rapoport D A, Fabian S A, Siegel S J, Wand G, Houslay M D, Kanes S J, Abel T, Abel T. Constitutive activation of Galphas within forebrain neurons causes deficits in sensorimotor gating because of PKA-dependent decreases in cAMP. Neuropsychopharmacology. 2007; 32(3): 577-88.
43. Kelly M P, Deadwyler S A. Acquisition of a novel behavior induces higher levels of arc mRNA than does overtrained performance. Neuroscience. 2002; 110(4): 617-26. doi: Pii S0306-4522(01) 00605-4
A) Doi 10.1016/S0306-4522(01) 00605-4. PubMed PMID: WOS: 000175245300003.
44. Kelly M P, Deadwyler S A. Experience-dependent regulation of the immediate-early gene arc differs across brain regions. The Journal of neuroscience: the official journal of the Society for Neuroscience. 2003; 23(16): 6443-51. Epub 2003 Jul. 25. PubMed PMID: 12878684.
45. Pathak G, Ibrahim B A, McCarthy S A, Baker K, Kelly M P. Amphetamine sensitization in mice is sufficient to produce both manic- and depressive-related behaviors as well as changes in the functional connectivity of corticolimbic structures. Neuropharmacology. 2015; 95:434-47. doi: 10.1016/j.neuropharm.2015.04.026. PubMed PMID: 25959066.
46. Kelly M P. Cyclic nucleotide signaling changes associated with normal aging and age-related diseases of the brain. Cellular signalling. 2018; 42:281-91. doi: 10.1016/j.cellsig.2017.11.004. PubMed PMID: 29175000; PubMed Central PMCID: PMC5732030.
47. Ishizuka K, Kamiya A, Oh E C, Kanki H, Seshadri S, Robinson J F, Murdoch H, Dunlop A J, Kubo K, Furukori K, Huang B, Zeledon M, Hayashi-Takagi A, Okano H, Nakajima K, Houslay M D, Katsanis N, Sawa A. DISC1-dependent switch from progenitor proliferation to migration in the developing cortex. Nature. 2011; 473(7345): 92-6. doi: 10.1038/nature09859. PubMed PMID: 21471969; PubMed Central PMCID: PMC3088774.
48. Houslay M D. Underpinning compartmentalised cAMP signalling through targeted CAMP breakdown. Trends Biochem Sci. 2010; 35(2): 91-100. doi: 10.1016/j.tibs.2009.09.007. PubMed PMID: 19864144.
49. Kokkonen K, Kass D A. Nanodomain Regulation of Cardiac Cyclic Nucleotide Signaling by Phosphodiesterases. Annu Rev Pharmacol Toxicol. 2017; 57:455-79. doi: 10.1146/annurev-pharmtox-010716-104756. PubMed PMID: 27732797.
50. Rahman S, Li P P, Young L T, Kofman O, Kish S J, Warsh J J. Reduced [3H]cyclic AMP binding in postmortem brain from subjects with bipolar affective disorder. Journal of neurochemistry. 1997; 68(1): 297-304. PubMed PMID: 8978738.
51. Fields A, Li P P, Kish S J, Warsh J J. Increased cyclic AMP-dependent protein kinase activity in postmortem brain from patients with bipolar affective disorder. Journal of neurochemistry. 1999; 73(4): 1704-10. PubMed PMID: 10501218.
52. Chang A, Li P P, Warsh J J. Altered cAMP-dependent protein kinase subunit immunolabeling in post-mortem brain from patients with bipolar affective disorder. [erratum appears in J Neurochem. 2003 April; 85(1): 286.]. Journal of neurochemistry. 2003; 84(4): 781-91.
53. Bonkale W L, Winblad B, Ravid R, Cowburn R F. Reduced nitric oxide responsive soluble guanylyl cyclase activity in the superior temporal cortex of patients with Alzheimer's disease. Neuroscience letters. 1995; 187(1): 5-8. PubMed PMID: 7617301.
54. Salpietro V, Perez Duenas B, Nakashima K, San Antonio M V, Manole A, Efthymiou S, Bettencourt C, Mencacci N E, Klein C, Kelly M P, Davies C H, Kimura H, Macaya A, Houlden H. A homozygous loss-of-function mutation in PDE2A associated to infantile-onset hereditary chorea Movement disorders: official journal of the Movement Disorder Society. in press: DOI: 10.1002/mds.27286.
55. Small S A, Schobel S A, Buxton R B, Witter M P, Barnes C A. A pathophysiological framework of hippocampal dysfunction in ageing and disease. Nature reviews Neuroscience. 2011; 12(10): 585-601. doi: 10.1038/nrn3085. PubMed PMID: 21897434; PubMed Central PMCID: PMC3312472.
56. Geiller T, Royer S, Choi J S. Segregated Cell Populations Enable Distinct Parallel Encoding within the Radial Axis of the CA1 Pyramidal Layer. Experimental neurobiology. 2017; 26(1): 1-10. doi: 10.5607/en.2017.26.1.1. PubMed PMID: 28243162; PubMed Central PMCID: PMC5326710.
57. Cembrowski M S, Bachman J L, Wang L, Sugino K, Shields B C, Spruston N. Spatial Gene-Expression Gradients Underlie Prominent Heterogeneity of CA1 Pyramidal Neurons. Neuron. 2016; 89(2): 351-68. doi: 10.1016/j.neuron.2015.12.013. PubMed PMID: 26777276.
58. Okuyama T, Kitamura T, Roy D S, Itohara S, Tonegawa S. Ventral CA1 neurons store social memory. Science. 2016; 353(6307): 1536-41. doi: 10.1126/science.aaf7003. PubMed PMID: 27708103; PubMed Central PMCID: PMC5493325.
59. Patel N, Klett J, Pilarzyk K, Lee D I, Kass D A, Menniti F, Kelly M P. Identification of new phosphodiesterase 9A (PDE9A) isoforms and how their expression and subcellular compartmentalization in brain changes across the lifespan. Neurobiology of aging. in press.
60. Loerch P M, Lu T, Dakin K A, Vann J M, Isaacs A, Geula C, Wang J, Pan Y, Gabuzda D H, Li C, Prolla T A, Yankner B A. Evolution of the aging brain transcriptome and synaptic regulation. PloS one. 2008; 3(10): e3329. doi: 10.1371/journal.pone.0003329. PubMed PMID: 18830410; PubMed Central PMCID: PMC2553198.
61. Kelly M P. Putting together the pieces of phosphodiesterase distribution patterns in the brain: A jigsaw puzzle of cyclic nucleotide regulation. In: Brandon N J, West A R, editors. Cyclic Nucleotide Phosphodiesterases in the Central Nervous System: From Biology to Disease. New Jersey: John Wiley & Sons, Inc; 2014.
62. Griffiths A M, J. H.; Suzuki, D. T.; Lewontin, R. C.; Gelbart, W. M. An Introduction to Genetic Analysis. 7th edition. New York: W. H. Freeman; 2000. Available from: http://www.ncbi.nlm.nih.gov/books/NBK21780.
63. Chhatwal J P, Hammack S E, Jasnow A M, Rainnie D G, Ressler K J. Identification of cell-type-specific promoters within the brain using lentiviral vectors. Gene therapy. 2007; 14(7): 575-83. doi: 10.1038/sj.gt.3302898. PubMed PMID: 17235291; PubMed Central PMCID: PMC2442925.
64. Gaveriaux-Ruff C, Kieffer B L. Conditional gene targeting in the mouse nervous system: Insights into brain function and diseases. Pharmacology & therapeutics. 2007; 113(3): 619-34. doi: 10.1016/j.pharmthera.2006.12.003. PubMed PMID: 17289150.
65. Lu J Y, Sewer M B. p54nrb/NONO regulates cyclic AMP-dependent glucocorticoid production by modulating phosphodiesterase mRNA splicing and degradation. Molecular and cellular biology. 2015; 35(7): 1223-37. doi: 10.1128/MCB.00993-14. PubMed PMID: 25605330; PubMed Central PMCID: PMC4355540.
66. Huang C J, Das U, Xie W, Ducasse M, Tucker H O. Altered stoichiometry and nuclear delocalization of NonO and PSF promote cellular senescence. Aging. 2016; 8(12): 3356-74. doi: 10.18632/aging.101125. PubMed PMID: 27992859; PubMed Central PMCID: PMC5270673.
67. Koppel I, Aid-Pavlidis T, Jaanson K, Sepp M, Pruunsild P, Palm K, Timmusk T. Tissue-specific and neural activity-regulated expression of human BDNF gene in BAC transgenic mice. BMC neuroscience. 2009; 10:68. doi: 10.1186/1471-2202-10-68. PubMed PMID: 19555478; PubMed Central PMCID: PMC2708170.
68. Koppel I, Aid-Pavlidis T, Jaanson K, Sepp M, Palm K, Timmusk T. BAC transgenic mice reveal distal cis-regulatory elements governing BDNF gene expression. Genesis. 2010; 48(4): 214-9. doi: 10.1002/dvg.20606. PubMed PMID: 20186743; PubMed Central PMCID: PMC2978326.
69. Inukai S, de Lencastre A, Turner M, Slack F. Novel microRNAs differentially expressed during aging in the mouse brain. PloS one. 2012; 7(7): e40028. doi: 10.1371/journal.pone.0040028. PubMed PMID: 22844398; PubMed Central PMCID: PMC3402511.
70. Kelly M P, Cheung Y F, Favilla C, Siegel S J, Kanes S J, Houslay M D, Abel T, Kelly M P, Cheung Y F, Favilla C, Siegel S J, Kanes S J, Houslay M D, Abel T. Constitutive activation of the G-protein subunit Galphas within forebrain neurons causes PKA-dependent alterations in fear conditioning and cortical Arc mRNA expression. Learning & Memory. 2008; 15(2): 75-83.
71. Pathak, G., et al., PDE11A negatively regulates lithium responsivity. Mol Psychiatry, 2017. 22(12): p. 1714-1724.
72. Pilarzyk K, P. L., Capell W R, Burbano S, Davis J, Fisher J L, Gorny N, Petrolle S, Kelly M P, Proteinopathies in hippocampal PDE11A4 promote age-related cognitive decline of social associative memories. In Preparation.
73. Smith, A. J., et al., A genetic basis for friendship? Homophily for membrane-associated PDE11A-CAMP-CREB signaling in CA1 of hippocampus dictates mutual social preference in male and female mice. Mol Psychiatry, 2021.
74. Zhang, C., et al., RNA interference-mediated knockdown of long-form phosphodiesterase-4D (PDE4D) enzyme reverses amyloid-beta42-induced memory deficits in mice. J Alzheimers Dis, 2014. 38(2): p. 269-80.
75. Bonkale, W. L., et al., A quantitative autoradiographic study of [3H] cAMP binding to cytosolic and particulate protein kinase A in post-mortem brain staged for Alzheimer's disease neurofibrillary changes and amyloid deposits. Brain Res, 1999. 818(2): p. 383-96.
76. Kelly, M. P., Cyclic nucleotide signaling changes associated with normal aging and age-related diseases of the brain. Cell Signal, 2018. 42: p. 281-291.
77. Baillie, G. S., G. S. Tejeda, and M. P. Kelly, Therapeutic targeting of 3′,5′-cyclic nucleotide phosphodiesterases: inhibition and beyond. Nat Rev Drug Discov, 2019. 18(10): p. 770-796.
78. Kelly, M. P., Pde11a, in Encyclopedia of Signaling Molecules, S. Choi, Editor. 2018, Springer International Publishing: Cham. p. 3804-3826.
79. Patel, N. S., et al., Identification of new PDE9A isoforms and how their expression and subcellular compartmentalization in the brain change across the life span. Neurobiol Aging, 2018. 65: p. 217-234.
80. Houslay, M. D., Underpinning compartmentalised cAMP signalling through targeted CAMP breakdown. Trends Biochem Sci, 2010. 35(2): p. 91-100.
81. Kokkonen, K. and D. A. Kass, Nanodomain Regulation of Cardiac Cyclic Nucleotide Signaling by Phosphodiesterases. Annu Rev Pharmacol Toxicol, 2017. 57: p. 455-479.
82. Kelly, M. P., et al., Select 3′,5′-cyclic nucleotide phosphodiesterases exhibit altered expression in the aged rodent brain. Cell Signal, 2014. 26(2): p. 383-97.
83. Rahman, S., et al., Reduced [3H]cyclic AMP binding in postmortem brain from subjects with bipolar affective disorder. J Neurochem, 1997. 68(1): p. 297-304.
84. Fields, A., et al., Increased cyclic AMP-dependent protein kinase activity in postmortem brain from patients with bipolar affective disorder. J Neurochem, 1999. 73(4): p. 1704-10.
85. Chang, A., P. P. Li, and J. J. Warsh, Altered cAMP-dependent protein kinase subunit immunolabeling in post-mortem brain from patients with bipolar affective disorder. [erratum appears in J Neurochem. 2003 April; 85(1): 286.]. Journal of Neurochemistry, 2003. 84(4): p. 781-791.
86. Bonkale, W. L., et al., Reduced nitric oxide responsive soluble guanylyl cyclase activity in the superior temporal cortex of patients with Alzheimer's disease. Neurosci Lett, 1995. 187(1): p. 5-8.
87. Qin, W., et al., Exome sequencing revealed PDE11A as a novel candidate gene for early-onset Alzheimer's disease. Hum Mol Genet, 2021. 30(9): p. 811-822.
88. Kelly, M. P., Does phosphodiesterase 11A (PDE11A) hold promise as a future therapeutic target? Curr Pharm Des, 2015. 21(3): p. 389-416.
89. Weeks, J. L., et al., High biochemical selectivity of tadalafil, sildenafil and vardenafil for human phosphodiesterase 5A1 (PDE5) over PDE11A4 suggests the absence of PDE11A4 cross-reaction in patients. Int J Impot Res, 2005. 17(1): p. 5-9.
90. Gross-Langenhoff, M., et al., The properties of phosphodiesterase 11A4 GAF domains are regulated by modifications in its N-terminal domain. FEBS J, 2008. 275(8): p. 1643-50.
91. Hegde, S., et al., Phosphodiesterase 11A (PDE11A), Enriched in Ventral Hippocampus Neurons, is Required for Consolidation of Social but not Nonsocial Memories in Mice. Neuropsychopharmacology, 2016. 41(12): p. 2920-2931.
92. Pilarzyk, K., et al., Loss of Function of Phosphodiesterase 11A4 Shows that Recent and Remote Long-Term Memories Can Be Uncoupled. Curr Biol, 2019. 29(14): p. 2307-2321 e5.
93. Hegde, S., et al., PDE11A regulates social behaviors and is a key mechanism by which social experience sculpts the brain. Neuroscience, 2016. 335: p. 151-69.
94. Pilarzyk, K., et al., The Role of PDE11A4 in Social Isolation-Induced Changes in Intracellular Signaling and Neuroinflammation. Front Pharmacol, 2021. 12: p. 749628.
95. Kelly, M. P., et al., Phosphodiesterase 11A in brain is enriched in ventral hippocampus and deletion causes psychiatric disease-related phenotypes. Proc Natl Acad Sci USA, 2010. 107(18): p. 8457-62.
96. Porcher, L., et al., Aging triggers an upregulation of a multitude of cytokines in the male and especially the female rodent hippocampus but more discrete changes in other brain regions. J Neuroinflammation, 2021. 18(1): p. 219.
97. Pathak, G., et al., Amphetamine sensitization in mice is sufficient to produce both manic- and depressive-related behaviors as well as changes in the functional connectivity of corticolimbic structures. Neuropharmacology, 2015. 95: p. 434-447.
98. Kelly, M. P., et al., The supra-additive hyperactivity caused by an amphetamine-chlordiazepoxide mixture exhibits an inverted-U dose response: negative implications for the use of a model in screening for mood stabilizers. Pharmacol Biochem Behav, 2009. 92(4): p. 649-54.
99. Galef, B. G., Jr., et al., Carbon disulfide: a semiochemical mediating socially-induced diet choice in rats. Physiol Behav, 1988. 42(2): p. 119-24.
100. Munger, S. D., et al., An olfactory subsystem that detects carbon disulfide and mediates food-related social learning. Curr Biol, 2010. 20(16): p. 1438-44.
101. Charych, E. I., et al., Interplay of palmitoylation and phosphorylation in the trafficking and localization of phosphodiesterase 10A: implications for the treatment of schizophrenia. J Neurosci, 2010. 30(27): p. 9027-37.
102. Yuasa, K., et al., Genomic organization of the human phosphodiesterase PDE11A gene. Evolutionary relatedness with other PDEs containing GAF domains. Eur J Biochem, 2001. 268(1): p. 168-78.
103. Yanar, K., et al., Novel biomarkers for the evaluation of aging-induced proteinopathies. Biogerontology, 2020. 21(5): p. 531-548.
104. Chalimoniuk, M. and J. B. Strosznajder, Aging modulates nitric oxide synthesis and cGMP levels in hippocampus and cerebellum. Effects of amyloid beta peptide. Mol Chem Neuropathol, 1998. 35(1-3): p. 77-95.
105. Vallebuona, F. and M. Raiteri, Age-related changes in the NMDA receptor/nitric oxide/cGMP pathway in the hippocampus and cerebellum of freely moving rats subjected to transcerebral microdialysis. Eur J Neurosci, 1995. 7(4): p. 694-701.
106. Moloney, C. M., V. J. Lowe, and M. E. Murray, Visualization of neurofibrillary tangle maturity in Alzheimer's disease: A clinicopathologic perspective for biomarker research. Alzheimers Dement, 2021.
107. Kleppisch, T. and R. Feil, cGMP signalling in the mammalian brain: role in synaptic plasticity and behaviour. Handb Exp Pharmacol, 2009(191): p. 549-79.
108. Cossenza, M., et al., Nitric oxide in the nervous system: biochemical, developmental, and neurobiological aspects. Vitam Horm, 2014. 96: p. 79-125.
109. Puzzo, D., et al., Phosphodiesterase 5 inhibition improves synaptic function, memory, and amyloid-beta load in an Alzheimer's disease mouse model. J Neurosci, 2009. 29(25): p. 8075-86.
110. Fiorito, J., et al., Identification of a Novel 1,2,3,4-Tetrahydrobenzo[b][1,6]naphthyridine Analogue as a Potent Phosphodiesterase 5 Inhibitor with Improved Aqueous Solubility for the Treatment of Alzheimer's Disease. J Med Chem, 2017. 60(21): p. 8858-8875.
111. Fiorito, J., et al., Synthesis of quinoline derivatives: discovery of a potent and selective phosphodiesterase 5 inhibitor for the treatment of Alzheimer's disease. Eur J Med Chem, 2013. 60: p. 285-94.
112. Palmeri, A., et al., Inhibition of phosphodiesterase-5 rescues age-related impairment of synaptic plasticity and memory. Behav Brain Res, 2013. 240: p. 11-20.
113. Hill, S. M., S. Hanzen, and T. Nystrom, Restricted access: spatial sequestration of damaged proteins during stress and aging. EMBO Rep, 2017. 18(3): p. 377-391.
114. Christian, F., et al., p62 (SQSTM1) and cyclic AMP phosphodiesterase-4A4 (PDE4A4) locate to a novel, reversible protein aggregate with links to autophagy and proteasome degradation pathways. Cell Signal, 2010. 22(10): p. 1576-96.

1. An isolated fragment of PDE11A4 comprising a GAF-B binding sequence and comprising or consisting of a polypeptide sequence of SEQ ID NO: 1, or a sequence having at least about 70%, at least about 75%, at least about 80%, at least about 85%, at least about 87%, at least about 89%, at least about 90%, at least about 91%, at least about 92%, at least about 93%, at least about 94%, at least about 95%, at least about 96%, at least about 97%, at least about 98%, or at least about 99% identity thereto.

2. The isolated fragment of claim 1, wherein the isolated fragment comprises and or consists of a polypeptide sequence of SEQ ID NO: 1.

3. An isolated fragment of PDE11A4 comprising a GAF-B binding sequence and comprising or consisting of a polypeptide sequence of SEQ ID NO: 1 or SEQ ID NO: 2, or a sequence having at least about 70%, at least about 75%, at least about 80%, at least about 85%, at least about 87%, at least about 89%, at least about 90%, at least about 91%, at least about 92%, at least about 93%, at least about 94%, at least about 95%, at least about 96%, at least about 97%, at least about 98%, or at least about 99% identity thereto.

4. The isolated fragment of claim 3, wherein the isolated fragment comprises and or consists of a polypeptide sequence of SEQ ID NO: 1 or SEQ ID NO: 2.

5. A polynucleotide encoding an isolated fragment of PDE11A4 comprising a GAF-B binding sequence having an polypeptide sequence of SEQ ID NO: 1, or a sequence having at least about 70%, at least about 75%, at least about 80%, at least about 85%, at least about 87%, at least about 89%, at least about 90%, at least about 91%, at least about 92%, at least about 93%, at least about 94%, at least about 95%, at least about 96%, at least about 97%, at least about 98%, or at least about 99% identity thereto.

6. A polynucleotide encoding an isolated fragment of PDE11A4 comprising a GAF-B binding sequence having an polypeptide sequence of SEQ ID NO: 1 or SEQ ID NO: 2, or a sequence having at least about 70%, at least about 75%, at least about 80%, at least about 85%, at least about 87%, at least about 89%, at least about 90%, at least about 91%, at least about 92%, at least about 93%, at least about 94%, at least about 95%, at least about 96%, at least about 97%, at least about 98%, or at least about 99% identity thereto.

7. A vector comprising the nucleic acid of claim 5 or 6.

8. A host cell comprising the polynucleotide of claim 7.

9. The host cell of claim 8, wherein the cell is a mammalian cell.

10. The host cell of claim 9, wherein said host cell is a HT-22 cell, a CHO cell, a HEK-293 cell, or an Sp2.0 cell.

11. A pharmaceutical composition comprising the isolated fragment of any one of claims 1-4, and a physiologically compatible carrier medium.

12. A pharmaceutical composition comprising the isolated fragment of any one of claims 1-4, and a physiologically compatible carrier medium, wherein the amount of the isolated fragment in the composition is a therapeutically effective amount for the treatment or prevention of a disease or disorder alleviated by inhibiting PDE11A4 activity in a patient in need thereof.

13. The pharmaceutical composition of claim 11 or 12, wherein the disease or disorder is associated with cognitive decline.

14. The pharmaceutical composition of claim 13, wherein the disease or disorder is selected from dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), and cognitive decline associate with spatial memory, other depression-related behaviors, additional anxiety-related behaviors, sensorimotor gating, and social behaviors.

15. A method of treating or preventing a disease or disorder alleviated by inhibiting PDE11A4 activity in a patient in need thereof, comprising administering to the patient a therapeutically effective amount of the isolated fragment of any one of claims 1-4.

16. A method of treating or preventing a disease or disorder alleviated by inhibiting PDE11A4 activity in a patient in need thereof, comprising administering to the patient a therapeutically effective amount of a pharmaceutical composition of any one of claims 11-14.

17. The method of any one of claim 15 or 16, wherein the isolated fragment is administered in a dosage unit form.

18. The method of claim 17, wherein the dosage unit comprises a physiologically compatible carrier medium.

19. The method of any one of claims 15-18, wherein the disease or disorder is associated with cognitive decline.

20. The method of claim 19, wherein the disease or disorder is selected from dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), and cognitive decline associate with spatial memory, other depression-related behaviors, additional anxiety-related behaviors, sensorimotor gating, and social behaviors.
